(19) United States
(12) Patent Application Publication
(10) Pub. No.: US 2013/0112948 A1
(43) Pub. Date: May 9, 2013

(54) HETEROCYCLIC COMPOUND AND ORGANIC LIGHT-EMITTING DIODE INCLUDING THE SAME

(71) Applicants: Hye-Jin Jung, Yongin-City (KR); Seok-Hwan Hwang, Yongin-City (KR); Young-Kook Kim, Yongin-City (KR); Jin-O Lim, Yongin-City (KR); Sang-Hyun Han, Yongin-City (KR); Soo-Yon Kim, Yongin-City (KR); Jin-Young Yun, Yongin-City (KR); Jong-Hyuk Lee, Yongin-City (KR)

(72) Inventors: Hye-Jin Jung, Yongin-City (KR); Seok-Hwan Hwang, Yongin-City (KR); Young-Kook Kim, Yongin-City (KR); Jin-O Lim, Yongin-City (KR); Sang-Hyun Han, Yongin-City (KR); Soo-Yon Kim, Yongin-City (KR); Jin-Young Yun, Yongin-City (KR); Jong-Hyuk Lee, Yongin-City (KR)

(73) Assignee: Samsung Mobile Display Co., Ltd., Yongin-City (KR)

(21) Appl. No.: 13/480,617

(22) Filed: May 25, 2012

(30) Foreign Application Priority Data
Nov. 3, 2011 (KR) 10-2011-0114118

(51) Int. Cl. C07D 209/08; H01L 51/54; C07D 401/12; C07D 403/12; C07D 409/10

(52) U.S. Cl. 257/40; 548/469; 548/442; 548/454; 546/277.4; 257/E51.026

(57) ABSTRACT

A heterocyclic compound represented by Formula 1A below and an organic light-emitting diode including the same:

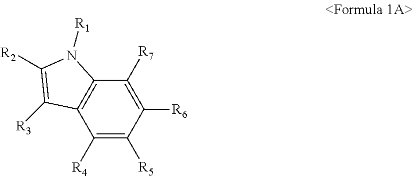

at least one of R₁ to R₇ is a group represented by Formula 1B below.

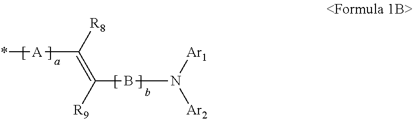

in Formulae 1A and 1B, R₁ to R₉, Ar₁, Ar₂, A, B, a and b are the same as described in the detailed description section of the present application. The organic light-emitting diode including an organic layer including the heterocyclic compound has a low driving voltage, high luminescence efficiency, and a long lifetime.

### CROSS-REFERENCE TO RELATED PATENT APPLICATION

This application makes reference to, incorporates the same herein, and claims all benefits accruing under 35 U.S.C. §119 from an application earlier filed in the Korean Intellectual Property Office on 3 Nov. 2011 and there duly assigned Serial No. 10-2011-0114118.

### BACKGROUND OF THE INVENTION

1. Field of the Invention

The present invention relates to heterocyclic compounds and organic light-emitting diodes including the same.

2. Description of the Related Art

Organic light emitting diodes are self-emission devices, have a wide viewing angle, a high contrast ratio, a short response time, and excellent brightness, driving voltage, and response speed characteristics, and enable generation of multi-color images.

In a typical organic light-emitting diode, an anode is formed on a substrate, and a hole transport layer, an emission layer, an electron transport layer, and a cathode are sequentially formed in this stated order on the anode. In this regard, the hole transport layer, the emission layer, and the electron transport layer are organic films including organic compounds.

A driving principle of an organic light-emitting diode having such a structure is as follows: when a voltage is applied between the anode and the cathode, holes injected from the anode pass the hole transport layer and migrate toward the emission layer, and electrons injected from the cathode pass the electron transport layer and migrate toward the emission layer, and the holes and electrons, which are carriers, are recombined in the emission layer to generate excitons, and then the excitons change from an excited state to a ground state, thereby generating light.

### SUMMARY OF THE INVENTION

Provided are novel heterocyclic compounds that have a low driving voltage, high brightness, high efficiency, and a long lifetime and are used in an organic light-emitting diode, and organic light-emitting diodes including an organic layer including the heterocyclic compounds.

According to an aspect of the present invention, a heterocyclic compound represented by Formula 1A below is provided:

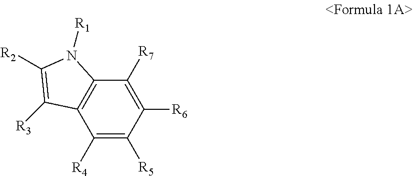

in Formula 1A, R₁ to R₇ are each independently a hydrogen atom, a deuterium atom, a halogen atom, a hydroxyl group, a cyano group, a nitro group, a carboxyl group, a substituted or unsubstituted C₁-C₃₀ alkyl group, a substituted or unsubstituted C₂-C₃₀ alkenyl group, a substituted or unsubstituted C₂-C₃₀ alkynyl group, a substituted or unsubstituted C₁-C₃₀ alkoxy group, a substituted or unsubstituted C₃-C₃₀ cycloalkyl group, a substituted or unsubstituted C₃-C₃₀ cycloalkenyl group, a substituted or unsubstituted C₅-C₃₀ aryl group, a substituted or unsubstituted C₂-C₃₀ heteroaryl group, a substituted or unsubstituted C₅-C₃₀ aryloxy group, a substituted or unsubstituted C₅-C₃₀ arylthio group, or a group represented by Formula 1B below;

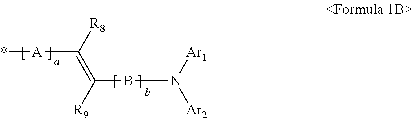

in Formula 1B, R₈ and R₉ are each independently a hydrogen atom, a deuterium atom, a halogen atom, a hydroxyl group, a cyano group, a nitro group, a carboxyl group, a substituted or unsubstituted C₁-C₃₀ alkyl group, a substituted or unsubstituted C₂-C₃₀ alkenyl group, a substituted or unsubstituted C₂-C₃₀ alkynyl group, a substituted or unsubstituted C₁-C₃₀ alkoxy group, a substituted or unsubstituted C₃-C₃₀ cycloalkyl group, a substituted or unsubstituted C₃-C₃₀ cycloalkenyl group, a substituted or unsubstituted C₅-C₃₀ aryl group, a substituted or unsubstituted C₂-C₃₀ heteroaryl group, a substituted or unsubstituted C₅-C₃₀ aryloxy group, or a substituted or unsubstituted C₅-C₃₀ arylthio group;

at least one of R₁ to R₇ is a group represented by Formula 1B above;

Ar₁ and Ar₂ are each independently a substituted or unsubstituted C₅-C₃₀ aryl group, or a substituted or unsubstituted C₃-C₃₀ heteroaryl group;

A and B are each a bivalent linker, and are each independently a substituted or unsubstituted C₅-C₃₀ arylene group, or a substituted or unsubstituted C₃-C₃₀ heteroarylene group; and

a is an integer of 0 to 3, and if a is 2 or more, 2 or more A are identical to or different from each other, and b is an integer of 0 to 3, and if b is 2 or more, 2 or more B are identical to or different from each other, and * indicates a binding site.

According to an aspect of the present invention, an organic light-emitting diode includes a first electrode; a second electrode disposed facing the first electrode; and an organic layer interposed between the first electrode and the second electrode, wherein the organic layer may include at least one layer and one or more kinds of the heterocyclic compound represented by Formula 1A.

### BRIEF DESCRIPTION OF THE DRAWINGS

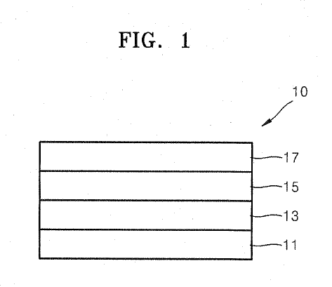

These and/or other aspects will become apparent and more readily appreciated from the following description of the embodiments, taken in conjunction with FIG. 1 that is a schematic sectional view of an organic light-emitting diode according to an embodiment of the present invention.

### DETAILED DESCRIPTION

Reference will now be made in detail to embodiments, examples of which are illustrated in the accompanying drawings, wherein like reference numerals refer to the like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

According to an aspect of the present invention, a heterocyclic compound represented by Formula 1A below is provided:

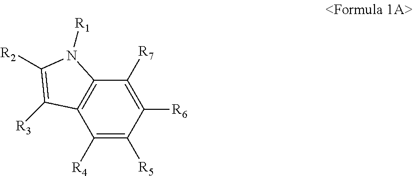

in Formula 1A, R₁ to R₇ are each independently a hydrogen atom, a deuterium atom, a halogen atom, a hydroxyl group, a cyano group, a nitro group, a carboxyl group, a substituted or unsubstituted C₁-C₃₀ alkyl group, a substituted or unsubstituted C₂-C₃₀ alkenyl group, a substituted or unsubstituted C₂-C₃₀ alkynyl group, a substituted or unsubstituted C₁-C₃₀ alkoxy group, a substituted or unsubstituted C₃-C₃₀ cycloalkyl group, a substituted or unsubstituted C₃-C₃₀ cycloalkenyl group, a substituted or unsubstituted C₅-C₃₀ aryl group, a substituted or unsubstituted C₂-C₃₀ heteroaryl group, a substituted or unsubstituted C₅-C₃₀ aryloxy group, a substituted or unsubstituted C₅-C₃₀ arylthio group, or a group represented by Formula 1B below:

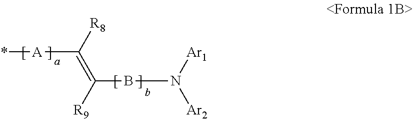

in Formula 1B, R₈ and R₉ are each independently a hydrogen atom, a deuterium atom, a halogen atom, a hydroxyl group, a cyano group, a nitro group, a carboxyl group, a substituted or unsubstituted C₁-C₃₀ alkyl group, a substituted or unsubstituted C₂-C₃₀ alkenyl group, a substituted or unsubstituted C₂-C₃₀ alkynyl group, a substituted or unsubstituted C₁-C₃₀ alkoxy group, a substituted or unsubstituted C₃-C₃₀ cycloalkyl group, a substituted or unsubstituted C₃-C₃₀ cycloalkenyl group, a substituted or unsubstituted C₅-C₃₀ aryl group, a substituted or unsubstituted C₂-C₃₀ heteroaryl group, a substituted or unsubstituted C₅-C₃₀ aryloxy group, or a substituted or unsubstituted C₅-C₃₀ arylthio group;

at least one of R₁ to R₇ is the group represented by Formula 1B above;

Ar₁ and Ar₂ are each independently a substituted or unsubstituted C₅-C₃₀ aryl group, or a substituted or unsubstituted C₃-C₃₀ heteroaryl group;

A and B are each a bivalent linker and are each independently a substituted or unsubstituted C₅-C₃₀ arylene group, or a substituted or unsubstituted C₃-C₃₀ heteroarylene group; and

a is an integer of 0 to 3, and if a is 2 or more, 2 or more A are identical to or different from each other, and b is an integer of 0 to 3, if b is 2 or more, 2 or more B are identical to or different from each other, and * indicates a binding site.

For example, Ar₁ and Ar₂ may be each independently a hydrogen atom, a deuterium atom, a halogen atom, a hydroxyl group, a cyano group, a nitro group, a carboxyl group, a substituted or unsubstituted methyl group, a substituted or unsubstituted ethyl group, a substituted or unsubstituted propyl group, a substituted or unsubstituted butyl group, a substituted or unsubstituted pentyl group, a substituted or unsubstituted ethenyl group, a substituted or unsubstituted phenyl group, a substituted or unsubstituted biphenyl group, a substituted or unsubstituted terphenyl group, a substituted or unsubstituted naphthyl group, a substituted or unsubstituted phenoxy, a substituted or unsubstituted fluorenyl group, a substituted or unsubstituted spiro-fluorenyl group, a substituted or unsubstituted carbazolyl group, a substituted or unsubstituted phenanthrenyl group, a substituted or unsubstituted pyranyl group, a substituted or unsubstituted pyridinyl group, a substituted or unsubstituted pyrimidinyl group, a substituted or unsubstituted diazinyl group, a substituted or unsubstituted triazinyl group, a substituted or unsubstituted quinolinyl group, a substituted or unsubstituted benzoimidazolyl group, a substituted or unsubstituted benzoxazolyl group, a substituted or unsubstituted pentalenyl group, a substituted or unsubstituted indenyl group, a substituted or unsubstituted azulenyl group, a substituted or unsubstituted heptalenyl group, a substituted or unsubstituted indacenyl group, a substituted or unsubstituted acenaphthyl group, a substituted or unsubstituted phenalenyl group, a substituted or unsubstituted phenanthridinyl group, a substituted or unsubstituted anthryl group, a substituted or unsubstituted fluoranthenyl group, a substituted or unsubstituted triphenylenyl group, a substituted or unsubstituted pyrenyl group, a substituted or unsubstituted chrysenyl group, a substituted or unsubstituted naphthacenyl group, a substituted or unsubstituted picenyl group, a substituted or unsubstituted perylenyl group, a substituted or unsubstituted pentaphenyl group, a substituted or unsubstituted hexacenyl group, a substituted or unsubstituted pyrrolyl group, a substituted or unsubstituted imidazolyl, a substituted or unsubstituted pyrazolyl group, a substituted or unsubstituted imidazopyridinyl group, a substituted or unsubstituted pyrazinyl group, a substituted or unsubstituted imidazopyrimidinyl group, a substituted or unsubstituted pyridazinyl group, a substituted or unsubstituted indolyl group, a substituted or unsubstituted indolizinyl group, a substituted or unsubstituted isoindolyl group, a substituted or unsubstituted pyridoindolyl, a substituted or unsubstituted indazolyl group, a substituted or unsubstituted carbazolyl group, a substituted or unsubstituted purinyl group, a substituted or unsubstituted benzoquinolinyl group, a substituted or unsubstituted phthalazinyl group, a substituted or unsubstituted naphthyridinyl group, a substituted or unsubstituted quinoxalinyl group, a substituted or unsubstituted quinazolinyl group, a substituted or unsubstituted cinnolinyl group, a substituted or unsubstituted phenazinyl group, a substituted or unsubstituted furanyl group, a substituted or unsubstituted benzofuranyl group, a substituted or unsubstituted dibenzofuranyl group, a substituted or unsubstituted thiophenyl group, a substituted or unsubstituted benzothiophenyl group, a substituted or unsubstituted dibenzothiophenyl group, a substituted or unsubstituted thiazolyl group, a substituted or unsubstituted isothiazolyl group, a substituted or unsubstituted benzothiazolyl group, a substituted or unsubstituted oxazolyl group, a substituted or unsubstituted isooxazolyl group, a substituted or unsubstituted oxadiazolyl group, a substituted or unsubstituted triazolyl group, or a substituted or unsubstituted tetrazolyl group.

For example, Ar₁ and Ar₂ may be each independently one of groups represented by Formulae 2A to 2I below, but are not limited thereto:

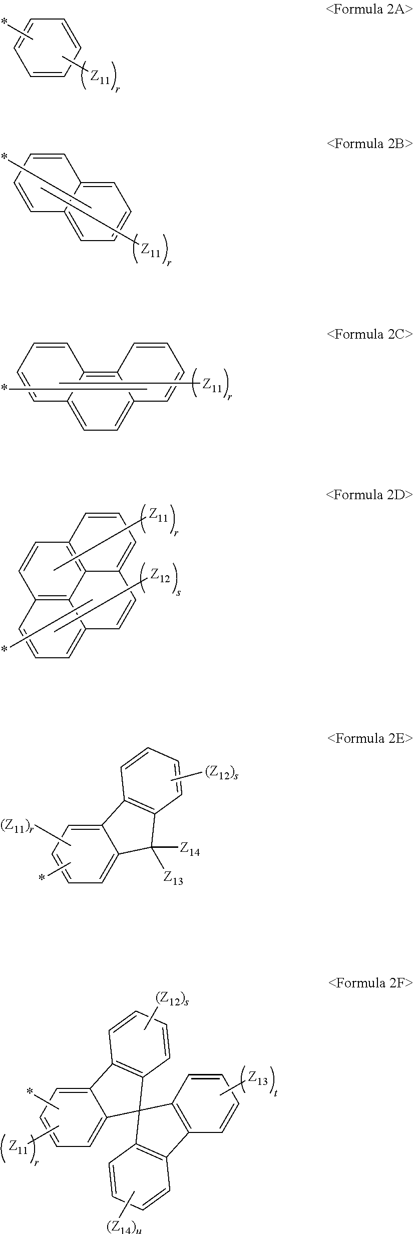

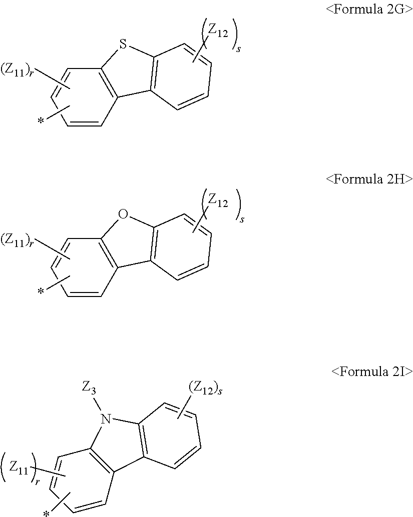

in Formula 2A to 2I,

Z₁₁, Z₁₂, Z₁₃ and Z₁₄ are each independently a hydrogen atom, a deuterium atom, a halogen atom, a hydroxyl group, a cyano group, a substituted or unsubstituted methyl group, a substituted or unsubstituted ethyl group, a substituted or unsubstituted propyl group, a substituted or unsubstituted butyl group, a substituted or unsubstituted methoxy group, a substituted or unsubstituted ethoxy group, a substituted or unsubstituted phenyl group, a substituted or unsubstituted naphthyl group, a substituted or unsubstituted anthryl group, a substituted or unsubstituted pyridinyl group, or a substituted or unsubstituted quinolinyl group, and

a plurality of each of Z₁₁, Z₁₂, Z₁₃ and Z₁₄ may be each independently identical to or different from each other, r is an integer of 1 to 9, s, t and u are each an integer of 1 to 4, and * indicates a binding site.

For example, Ar₁ and Ar₂ may each be independently one of groups represented by Formulae 3A to 3Q below, but are not limited thereto:

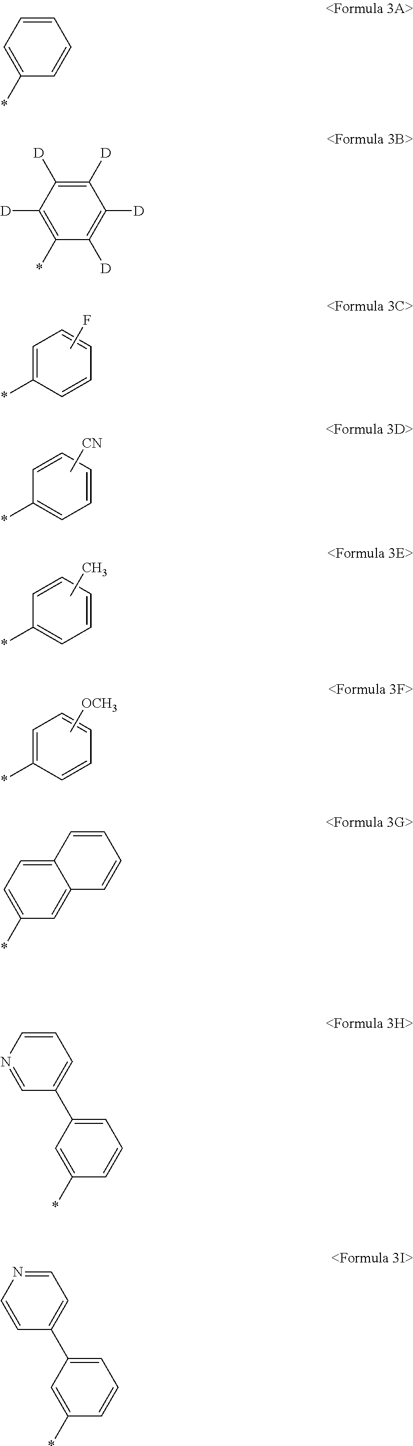

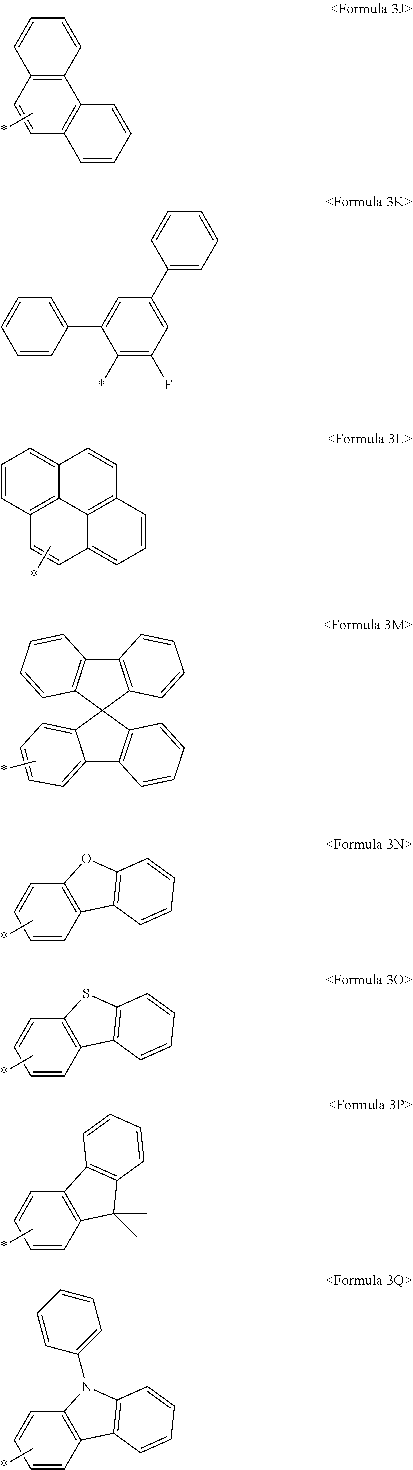

in Formulae 3A to 3Q, * indicates a binding site.

For example, A and B may be each independently a substituted or unsubstituted phenylene group, a substituted or unsubstituted pentalenylene group, a substituted or unsubstituted indenylene group, a substituted or unsubstituted naphthylene group, a substituted or unsubstituted azulenylene group, a substituted or unsubstituted heptalenylene group, a substituted or unsubstituted indacenylene group, a substituted or unsubstituted acenaphthylene group, a substituted or unsubstituted fluorenylene group, a substituted or unsubstituted phenalenylene group, a substituted or unsubstituted phenanthrenylene group, a substituted or unsubstituted anthrylene group, a substituted or unsubstituted fluoranthenylene group, a substituted or unsubstituted triphenylenylene group, a substituted or unsubstituted pyrenylene group, a substituted or unsubstituted chrysenylene group, a substituted or unsubstituted naphthacenylene group, a substituted or unsubstituted picenylene group, a substituted or unsubstituted perylenylene group, a substituted or unsubstituted pentaphenylene group, a substituted or unsubstituted hexacenylene group, a substituted or unsubstituted pyrrolylene group, a substituted or unsubstituted pyrazolylene group, a substituted or unsubstituted imidazolylene group, a substituted or unsubstituted imidazolinylene group, a substituted or unsubstituted imidazopyridinylene group, a substituted or unsubstituted imidazopyrimidinylene group, a substituted or unsubstituted pyridinylene group, a substituted or unsubstituted pyrazinylene group, a substituted or unsubstituted pyrimidinylene group, a substituted or unsubstituted indolylene group, a substituted or unsubstituted purinylene group, a substituted or unsubstituted quinolinylene group, a substituted or unsubstituted phthalazinylene group, a substituted or unsubstituted indolizinylene group, a substituted or unsubstituted naphthyridinylene group, a substituted or unsubstituted quinazolinylene group, a substituted or unsubstituted cinnolinylene group, a substituted or unsubstituted indazolylene group, a substituted or unsubstituted carbazolylene group, a substituted or unsubstituted phenazinylene group, a substituted or unsubstituted phenanthridinylene group, a substituted or unsubstituted pyranylene group, a substituted or unsubstituted chromenylene group, a substituted or unsubstituted furanylene group, a substituted or unsubstituted benzofuranylene group, a substituted or unsubstituted thiophenylene group, a substituted or unsubstituted benzothiophenylene group, a substituted or unsubstituted isothiazolylene group, a substituted or unsubstituted benzoimidazolylene group, a substituted or unsubstituted isoxazolylene group, a substituted or unsubstituted dibenzothiophenylene group, a substituted or unsubstituted dibenzofuranylene group, a substituted or unsubstituted triazinylene group, or a substituted or unsubstituted oxadiazolylene group.

For example, A and B may be each independently a substituted or unsubstituted phenylene group, a substituted or unsubstituted naphthylene group, a substituted or unsubstituted fluorenyl group, a substituted or unsubstituted carbazolylene group, a substituted or unsubstituted dibenzothiophenylene group, a substituted or unsubstituted pyridinylene group, a substituted or unsubstituted pyrimidinylene group, a substituted or unsubstituted triazinylene group, a substituted or unsubstituted anthrylene group, a substituted or unsubstituted phenanthrenylene group, a substituted or unsubstituted pyrenylene group, a substituted or unsubstituted chrycenyl group, a substituted or unsubstituted perylenylene group, a substituted or unsubstituted spiro-fluorenyl group, a substituted or unsubstituted furanylene group, a substituted or unsubstituted thiophenylene group, or a substituted or unsubstituted oxadiazolylene group.

For another example, A and B may be each independently one of groups represented by Formula 4A to 4E below, but are not limited thereto:

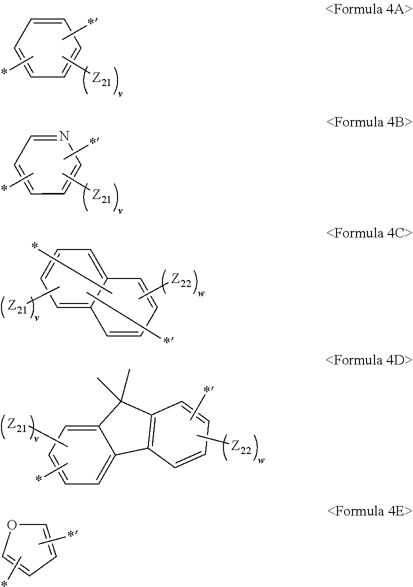

in Formulae 4A to 4E,

Z₂₁ and Z₂₂ are each independently a hydrogen atom, a deuterium atom, a halogen atom, a hydroxyl group, a cyano group, a substituted or unsubstituted methyl group, a substituted or unsubstituted ethyl group, a substituted or unsubstituted propyl group, a substituted or unsubstituted butyl group, a substituted or unsubstituted methoxy group, a substituted or unsubstituted ethoxy group, a substituted or unsubstituted phenyl group, a substituted or unsubstituted naphthyl group, or a substituted or unsubstituted pyridinyl group, and a plurality of each of Z₂₁ and Z₂₂ may be identical to or different from each other, v and w are each an integer of 1 to 4, * and *′ indicates a binding site.

For example, A and B may be each independently one of groups represented by Formulae 5A to 5F below, but are not limited thereto:

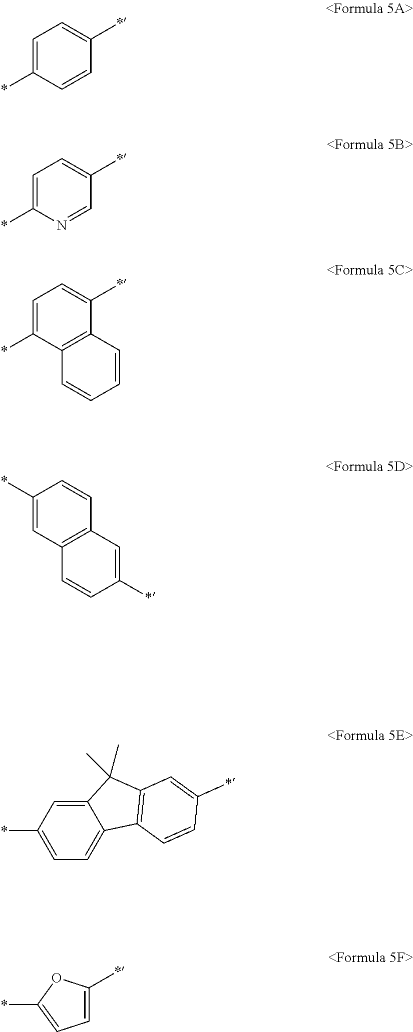

in Formulae 5A to 5F, * and indicate a binding site.

As for R₁ to R₇, R₁ to R₇ may be each independently a hydrogen atom, a deuterium atom, a halogen atom, a hydroxyl group, a cyano group, a nitro group, a carboxyl group, a substituted or unsubstituted C₁-C₂₀ alkyl group, a substituted or unsubstituted C₁-C₂₀alkoxy group, a substituted or unsubstituted phenyl group, a substituted or unsubstituted pentalenyl group, a substituted or unsubstituted indenyl group, a substituted or unsubstituted naphthyl group, a substituted or unsubstituted azulenyl group, a substituted or unsubstituted heptalenyl group, a substituted or unsubstituted indacenyl group, a substituted or unsubstituted acenaphthyl group, a substituted or unsubstituted fluorenyl group, a substituted or unsubstituted spiro-fluorenyl group, a substituted or unsubstituted phenalenyl group, a substituted or unsubstituted phenanthrenyl group, a substituted or unsubstituted anthryl group, a substituted or unsubstituted fluoranthenyl group, a substituted or unsubstituted triphenylenyl group, a substituted or unsubstituted pyrenyl group, a substituted or unsubstituted chrysenyl group, a substituted or unsubstituted naphthacenyl. substituted or unsubstituted picenyl group, a substituted or unsubstituted perylenyl group, a substituted or unsubstituted pentaphenyl group, a substituted or unsubstituted hexacenyl group, a substituted or unsubstituted pyrrolyl group, a substituted or unsubstituted imidazolyl group, a substituted or unsubstituted pyrazolyl group, a substituted or unsubstituted pyridinyl group, a substituted or unsubstituted pyrazinyl group, a substituted or unsubstituted pyrimidinyl group, a substituted or unsubstituted pyridazinyl group, a substituted or unsubstituted isoindolyl group, a substituted or unsubstituted indolyl group, a substituted or unsubstituted indazolyl group, a substituted or unsubstituted purinyl group, a substituted or unsubstituted quinolinyl group, a substituted or unsubstituted benzoquinolinyl group, a substituted or unsubstituted phthalazinyl group, a substituted or unsubstituted naphthyridinyl group, a substituted or unsubstituted quinoxalinyl group, a substituted or unsubstituted quinazolinyl group, a substituted or unsubstituted cinnolinyl group, a substituted or unsubstituted carbazolyl group, a substituted or unsubstituted phenanthridinyl group, a substituted or unsubstituted acridinyl group, a substituted or unsubstituted phenanthrolinyl group, a substituted or unsubstituted phenazinyl group, a substituted or unsubstituted benzoxazolyl group, a substituted or unsubstituted benzoimidazolyl group, a substituted or unsubstituted furanyl group, a substituted or unsubstituted benzofuranyl group, a substituted or unsubstituted thiophenyl group, a substituted or unsubstituted benzothiophenyl group, a substituted or unsubstituted thiazolyl group, a substituted or unsubstituted isothiazolyl group, a substituted or unsubstituted benzothiazolyl group, a substituted or unsubstituted isooxazolyl group, a substituted or unsubstituted oxazolyl group, a substituted or unsubstituted triazolyl group, a substituted or unsubstituted tetrazolyl group, a substituted or unsubstituted oxadiazolyl group, a substituted or unsubstituted triazinyl group, a substituted or unsubstituted benzoxazolyl group, a substituted or unsubstituted dibenzofuranyl group, or a substituted or unsubstituted dibenzothiophenyl or benzocarbazolyl.

For example, R₁ to R₇ may be each independently a hydrogen atom, a deuterium atom, a halogen atom, a hydroxyl group, a cyano group, a nitro group, a carboxyl group, a substituted or unsubstituted methyl group, a substituted or unsubstituted ethyl group, a substituted or unsubstituted propyl group, a substituted or unsubstituted butyl group, a substituted or unsubstituted pentyl group, a substituted or unsubstituted ethenyl group, a substituted or unsubstituted phenyl group, a substituted or unsubstituted biphenyl group, a substituted or unsubstituted terphenyl group, a substituted or unsubstituted naphthyl group, a substituted or unsubstituted anthryl group, a substituted or unsubstituted phenanthrenyl group, a substituted or unsubstituted pyrenyl group, a substituted or unsubstituted fluorenyl group, a substituted or unsubstituted carbazolyl group, a substituted or unsubstituted pyranyl group, a substituted or unsubstituted pyridinyl group, a substituted or unsubstituted pyrimidinyl group, a substituted or unsubstituted pyrazinyl group, a substituted or unsubstituted pyridazinyl group, a substituted or unsubstituted triazinyl group, a substituted or unsubstituted quinolinyl group, a substituted or unsubstituted benzoimidazolyl group, a substituted or unsubstituted benzoxazolyl group, a substituted or unsubstituted pentalenyl group, a substituted or unsubstituted indenyl group, a substituted or unsubstituted pyrrolyl group, a substituted or unsubstituted imidazolyl group, a substituted or unsubstituted pyrazolyl group, a group represented by N(Q₁)(Q₂), or the group represented by Formula 1B.

Q₁ and Q₂ may be each independently a hydrogen atom, a deuterium atom, a halogen atom, a hydroxyl group, a carboxyl group, a substituted or unsubstituted methyl group, a substituted or unsubstituted ethyl group, a substituted or unsubstituted propyl group, a substituted or unsubstituted butyl group, a substituted or unsubstituted pentyl group, a substituted or unsubstituted phenyl group, a substituted or unsubstituted naphthyl group, a substituted or unsubstituted anthryl group, a substituted or unsubstituted fluorenyl group, a substituted or unsubstituted carbazolyl group, or a substituted or unsubstituted pyridinyl.

For example ion, R₈ and R₉ may be each independently a hydrogen atom, a deuterium atom, a halogen atom, a hydroxyl group, a cyano group, a substituted or unsubstituted methyl group, a substituted or unsubstituted ethyl group, a substituted or unsubstituted propyl group, a substituted or unsubstituted butyl group, a substituted or unsubstituted pentyl group, a substituted or unsubstituted ethenyl group, a substituted or unsubstituted phenyl group, a substituted or unsubstituted naphthyl group, a substituted or unsubstituted anthryl group, or a substituted or unsubstituted phenanthrenyl.

For example, Ar₁ and Ar₂ may be each independently a substituted or unsubstituted phenyl group, a substituted or unsubstituted naphthyl group, a substituted or unsubstituted phenanthrenyl group, a substituted or unsubstituted pyrenyl group, a substituted or unsubstituted anthryl group, a substituted or unsubstituted fluorenyl group, a substituted or unsubstituted spiro-fluorenyl group, a substituted or unsubstituted dibenzothiophenyl group, a substituted or unsubstituted dibenzofuranyl group, a substituted or unsubstituted carbazolyl group, a substituted or unsubstituted pyridinyl group, a substituted or unsubstituted pyrimidinyl group, a substituted or unsubstituted triazinyl group, a substituted or unsubstituted chrysenyl group, a substituted or unsubstituted perylenyl group, or a substituted or unsubstituted oxadiazolyl;

For example, A and B may be each independently a substituted or unsubstituted phenylene group, a substituted or unsubstituted naphthylene group, a substituted or unsubstituted fluorenyl group, a substituted or unsubstituted carbazolylene group, a substituted or unsubstituted dibenzothiophenylene group, a substituted or unsubstituted pyridinylene group, a substituted or unsubstituted pyrimidinylene group, a substituted or unsubstituted triazinylene group, a substituted or unsubstituted anthrylene group, a substituted or unsubstituted phenanthrenylene group, a substituted or unsubstituted pyrenylene group, a substituted or unsubstituted chrycenyl group, a substituted or unsubstituted perylenylene group, a substituted or unsubstituted spiro-fluorenyl group, a substituted or unsubstituted furanylene group, a substituted or unsubstituted thiophenylene group, or a substituted or unsubstituted oxadiazolylene group;

a is an integer of 0 to 2, and if a is 2, two A may be identical to or different from each other, and b is an integer of 0 to 2, and if b is 2, two B may be identical to or different from each other.

R₁ to R₇ are each independently a hydrogen atom, a deuterium atom, a halogen atom, a substituted or unsubstituted methyl group, a substituted or unsubstituted ethyl group, a substituted or unsubstituted propyl group, a substituted or unsubstituted butyl group, the group represented by Formula 1B, or one of groups represented by Formulae 6A to 6D below, but are not limited thereto.

For example, R₈ and R₉ may be each independently a hydrogen atom, a deuterium atom, a halogen atom, a substituted or unsubstituted methyl group, a substituted or unsubstituted ethyl group, a substituted or unsubstituted propyl group, a substituted or unsubstituted butyl group, or one of the groups represented by Formulae 6A to 6D, but are not limited thereto:

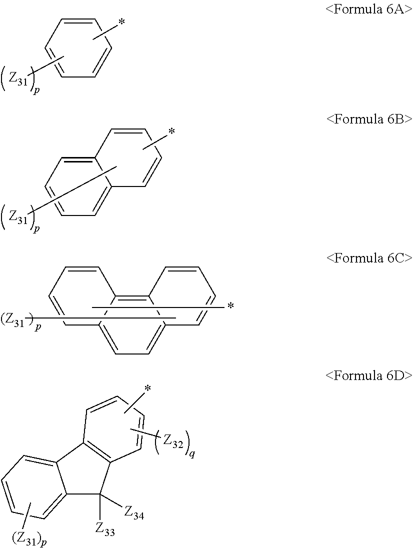

in Formulae 6A to 6D, Z₃₁, Z₃₂, Z₃₃ and Z₃₄ are each independently a hydrogen atom, a deuterium atom, a halogen atom, a hydroxyl group, a cyano group, a substituted or unsubstituted methyl group, a substituted or unsubstituted ethyl group, a substituted or unsubstituted propyl group, a substituted or unsubstituted butyl group, a substituted or unsubstituted methoxy group, a substituted or unsubstituted ethoxy group, a substituted or unsubstituted ethenyl group, a substituted or unsubstituted phenyl group, a substituted or unsubstituted naphthyl group, a substituted or unsubstituted anthryl group, a substituted or unsubstituted pyridinyl group, a substituted or unsubstituted carbazolyl group, or a substituted or unsubstituted quinolinyl group.

A plurality of each of Z₃₁ and Z₃₂ may be identical to or different from each other. p is an integer of 1 to 9, q is an integer of 1 to 4, and * indicates a binding site.

For example, R₁ to R₇ are each independently a hydrogen atom, a deuterium atom, a halogen atom, a substituted or unsubstituted methyl group, a substituted or unsubstituted ethyl group, a substituted or unsubstituted propyl group, a substituted or unsubstituted tertbutyl group, a cyano group, —CD₃, —CF₃, or one of groups represented by Formulae 7A to 7G below, but are not limited thereto.

In this regard, R₈ and R₉ are each independently a hydrogen atom, a deuterium atom, or one of the groups represented by Formulae 7A to 7G, but are not limited thereto:

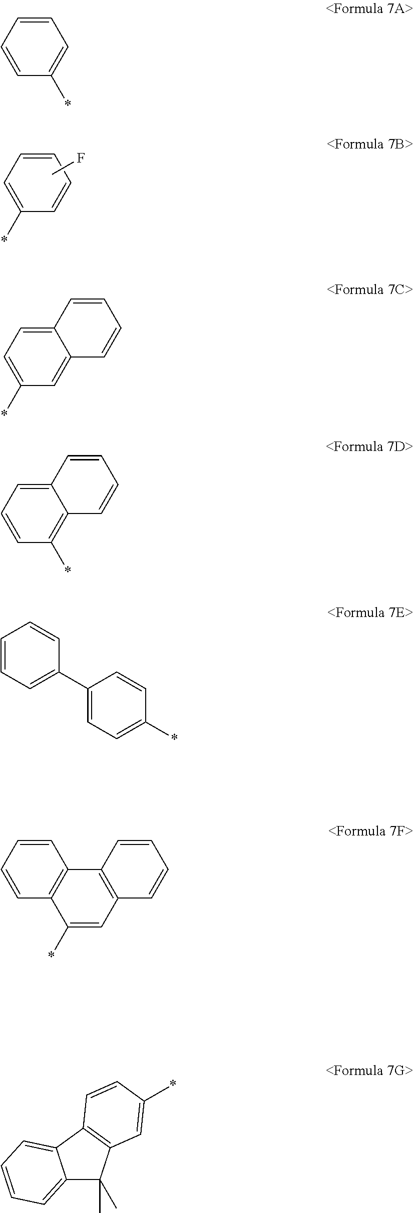

in Formulae 7A to 7G, * indicates a binding site.

The heterocyclic compound represented by Formula 1A may be one of Compounds 1 to 83 having the following structures, but are not limited thereto:

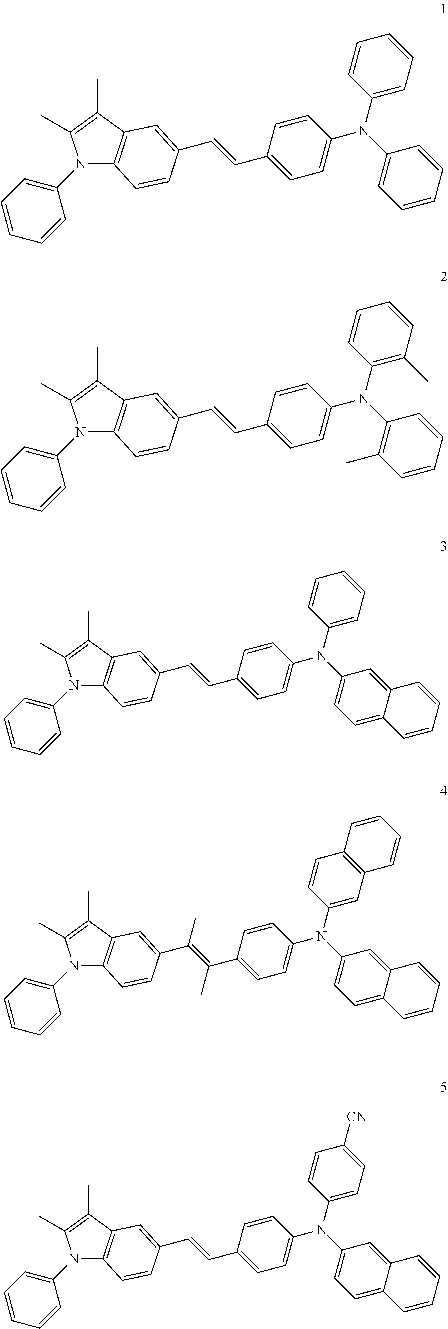 [SMILES: Cc1c(C)n(-c2ccccc2)c2ccc(/C=C/c3ccc(N(c4ccccc4)c4ccccc4)cc3)cc12 ; Cc1ccccc1N(c1ccc(/C=C/c2ccc3c(c2)c(C)c(C)n3-c2ccccc2)cc1)c1ccccc1C ; Cc1c(C)n(-c2ccccc2)c2ccc(/C=C/c3ccc(N(c4ccccc4)c4ccc5ccccc5c4)cc3)cc12 ; C/C(=C(/C)c1ccc2c(c1)c(C)c(C)n2-c1ccccc1)c1ccc(N(c2ccc3ccccc3c2)c2ccc3ccccc3c2)cc1 ; Cc1c(C)n(-c2ccccc2)c2ccc(/C=C/c3ccc(N(c4ccc(C#N)cc4)c4ccc5ccccc5c4)cc3)cc12]

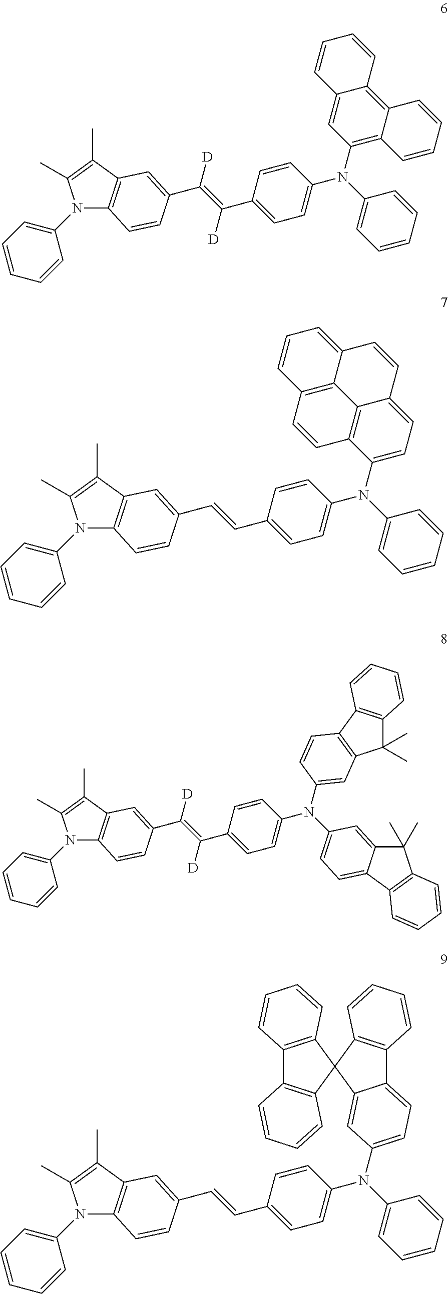 [SMILES: [2H]/C(=C(/[2H])c1ccc2c(c1)c(C)c(C)n2-c1ccccc1)c1ccc(N(c2ccccc2)c2cc3ccccc3c3ccccc23)cc1 ; Cc1c(C)n(-c2ccccc2)c2ccc(/C=C/c3ccc(N(c4ccccc4)c4ccc5ccc6cccc7ccc4c5c67)cc3)cc12 ; [2H]/C(=C(/[2H])c1ccc2c(c1)c(C)c(C)n2-c1ccccc1)c1ccc(N(c2ccc3c(c2)C(C)(C)c2ccccc2-3)c2ccc3c(c2)C(C)(C)c2ccccc2-3)cc1 ; Cc1c(C)n(-c2ccccc2)c2ccc(/C=C/c3ccc(N(c4ccccc4)c4ccc5c(c4)C4(c6ccccc6-c6ccccc64)c4ccccc4-5)cc3)cc12]

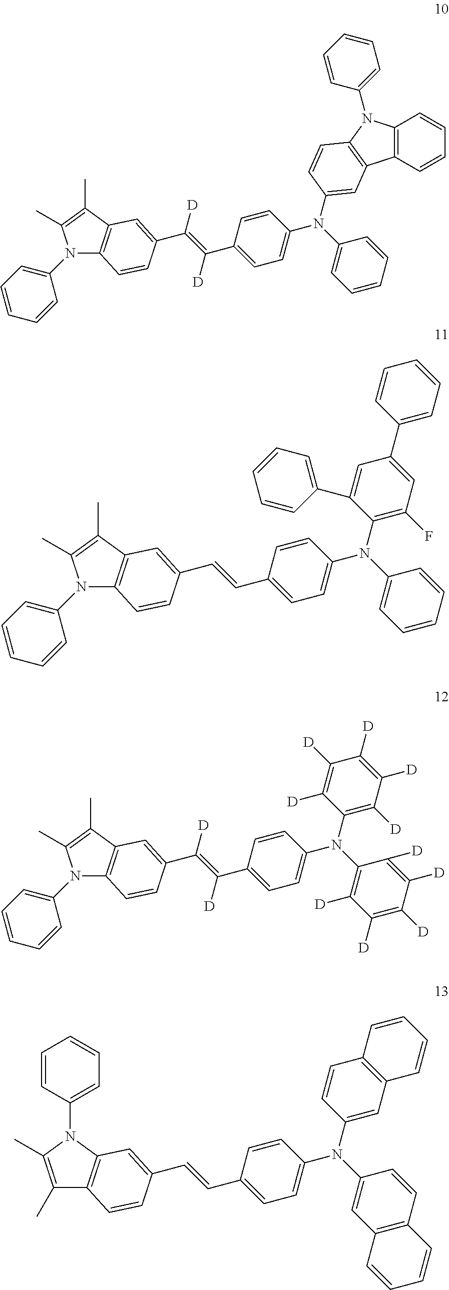 [SMILES: [2H]/C(=C(/[2H])c1ccc2c(c1)c(C)c(C)n2-c1ccccc1)c1ccc(N(c2ccccc2)c2ccc3c(c2)c2ccccc2n3-c2ccccc2)cc1 ; Cc1c(C)n(-c2ccccc2)c2ccc(/C=C/c3ccc(N(c4ccccc4)c4c(F)cc(-c5ccccc5)cc4-c4ccccc4)cc3)cc12 ; [2H]/C(=C(/[2H])c1ccc2c(c1)c(C)c(C)n2-c1ccccc1)c1ccc(N(c2c([2H])c([2H])c([2H])c([2H])c2[2H])c2c([2H])c([2H])c([2H])c([2H])c2[2H])cc1 ; Cc1c(C)n(-c2ccccc2)c2cc(/C=C/c3ccc(N(c4ccc5ccccc5c4)c4ccc5ccccc5c4)cc3)ccc12]

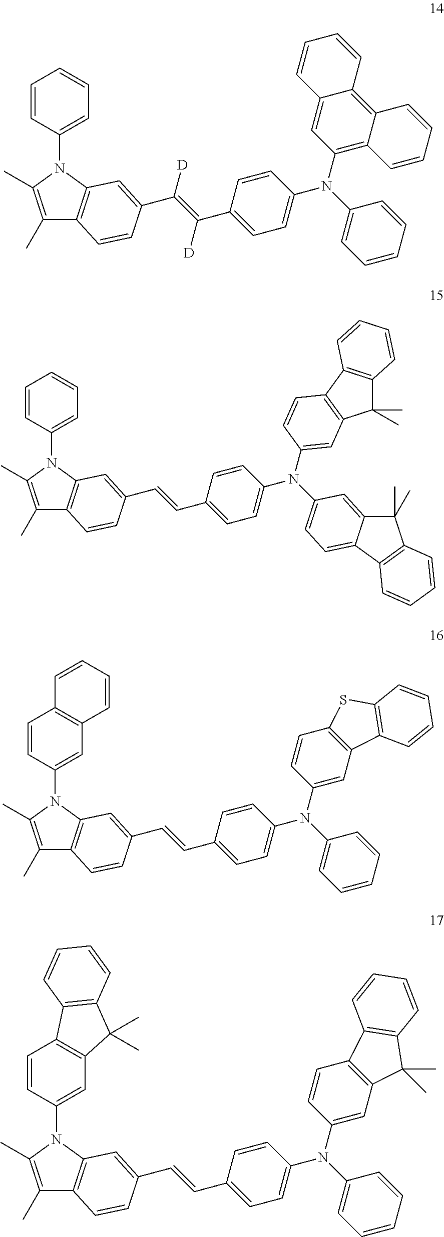

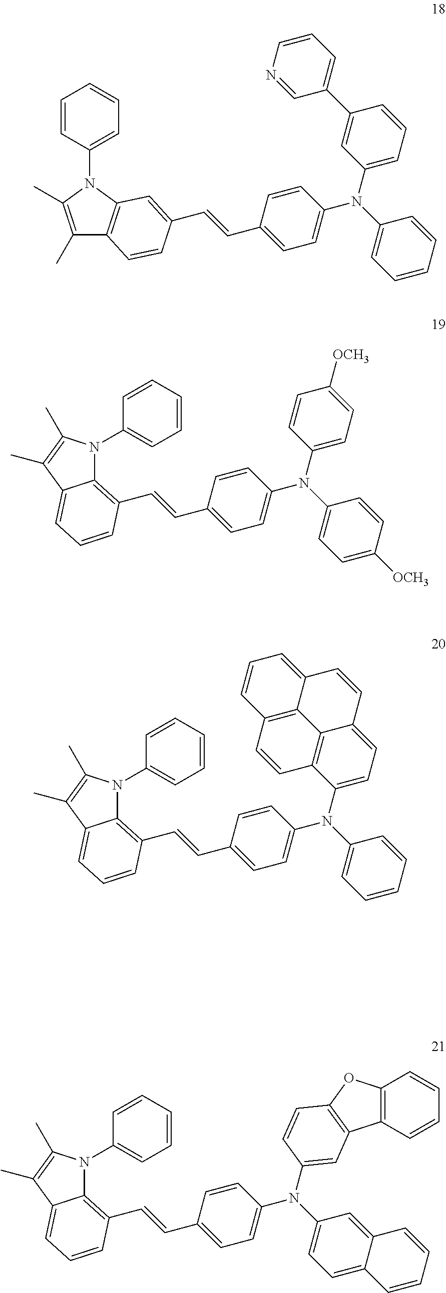 [SMILES: Cc1c(C)n(-c2ccccc2)c2cc(/C=C/c3ccc(N(c4ccccc4)c4cccc(-c5cccnc5)c4)cc3)ccc12 ; COc1ccc(N(c2ccc(/C=C/c3cccc4c(C)c(C)n(-c5ccccc5)c34)cc2)c2ccc(OC)cc2)cc1 ; Cc1c(C)n(-c2ccccc2)c2c(/C=C/c3ccc(N(c4ccccc4)c4ccc5ccc6cccc7ccc4c5c67)cc3)cccc12 ; Cc1c(C)n(-c2ccccc2)c2c(/C=C/c3ccc(N(c4ccc5ccccc5c4)c4ccc5oc6ccccc6c5c4)cc3)cccc12]

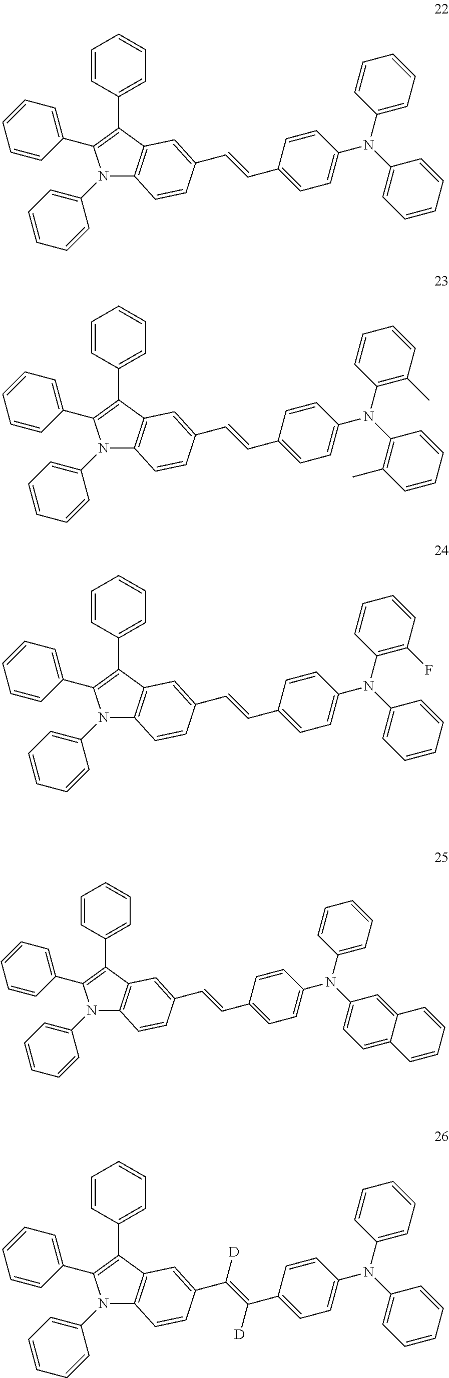 [SMILES: C(=C\c1ccc2c(c1)c(-c1ccccc1)c(-c1ccccc1)n2-c1ccccc1)/c1ccc(N(c2ccccc2)c2ccccc2)cc1 ; Cc1ccccc1N(c1ccc(/C=C/c2ccc3c(c2)c(-c2ccccc2)c(-c2ccccc2)n3-c2ccccc2)cc1)c1ccccc1C ; Fc1ccccc1N(c1ccccc1)c1ccc(/C=C/c2ccc3c(c2)c(-c2ccccc2)c(-c2ccccc2)n3-c2ccccc2)cc1 ; C(=C\c1ccc2c(c1)c(-c1ccccc1)c(-c1ccccc1)n2-c1ccccc1)/c1ccc(N(c2ccccc2)c2ccc3ccccc3c2)cc1 ; [2H]/C(=C(/[2H])c1ccc2c(c1)c(-c1ccccc1)c(-c1ccccc1)n2-c1ccccc1)c1ccc(N(c2ccccc2)c2ccccc2)cc1]

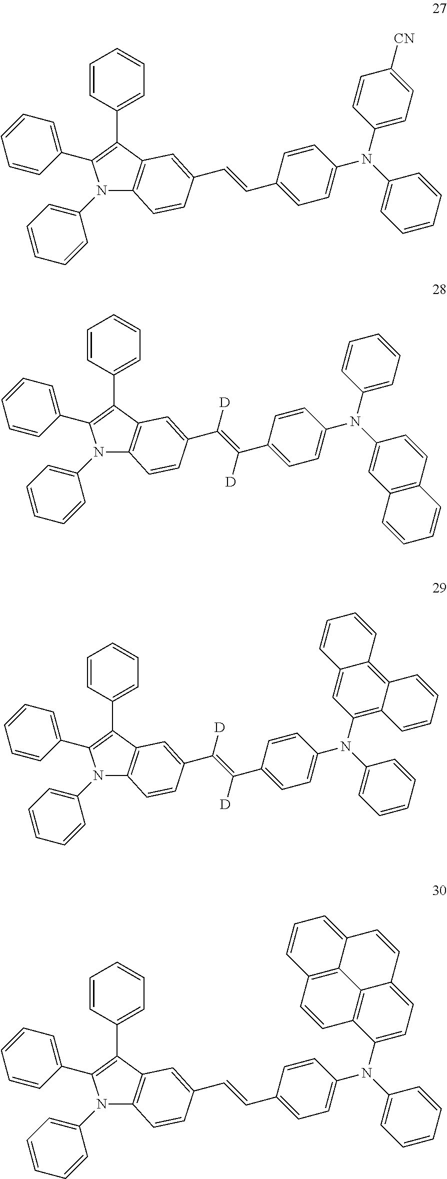 [SMILES: N#Cc1ccc(N(c2ccccc2)c2ccc(/C=C/c3ccc4c(c3)c(-c3ccccc3)c(-c3ccccc3)n4-c3ccccc3)cc2)cc1 ; [2H]/C(=C(/[2H])c1ccc2c(c1)c(-c1ccccc1)c(-c1ccccc1)n2-c1ccccc1)c1ccc(N(c2ccccc2)c2ccc3ccccc3c2)cc1 ; [2H]/C(=C(/[2H])c1ccc2c(c1)c(-c1ccccc1)c(-c1ccccc1)n2-c1ccccc1)c1ccc(N(c2ccccc2)c2cc3ccccc3c3ccccc23)cc1 ; C(=C\c1ccc2c(c1)c(-c1ccccc1)c(-c1ccccc1)n2-c1ccccc1)/c1ccc(N(c2ccccc2)c2ccc3ccc4cccc5ccc2c3c45)cc1]

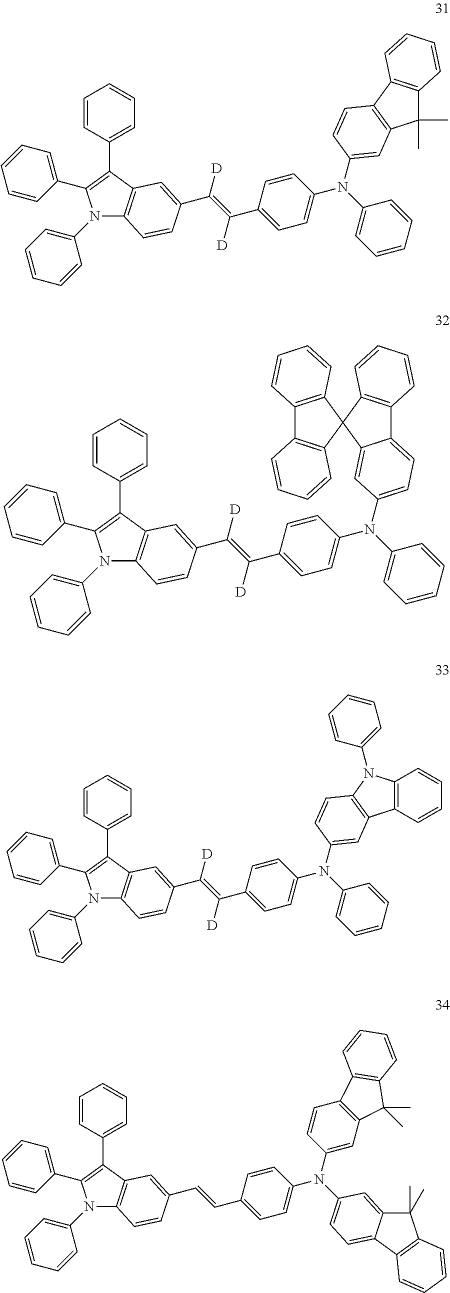 [SMILES: [2H]/C(=C(/[2H])c1ccc2c(c1)c(-c1ccccc1)c(-c1ccccc1)n2-c1ccccc1)c1ccc(N(c2ccccc2)c2ccc3c(c2)C(C)(C)c2ccccc2-3)cc1 ; [2H]/C(=C(/[2H])c1ccc2c(c1)c(-c1ccccc1)c(-c1ccccc1)n2-c1ccccc1)c1ccc(N(c2ccccc2)c2ccc3c(c2)C2(c4ccccc4-c4ccccc42)c2ccccc2-3)cc1 ; [2H]/C(=C(/[2H])c1ccc2c(c1)c(-c1ccccc1)c(-c1ccccc1)n2-c1ccccc1)c1ccc(N(c2ccccc2)c2ccc3c(c2)c2ccccc2n3-c2ccccc2)cc1 ; CC1(C)c2ccccc2-c2ccc(N(c3ccc(/C=C/c4ccc5c(c4)c(-c4ccccc4)c(-c4ccccc4)n5-c4ccccc4)cc3)c3ccc4c(c3)C(C)(C)c3ccccc3-4)cc21]

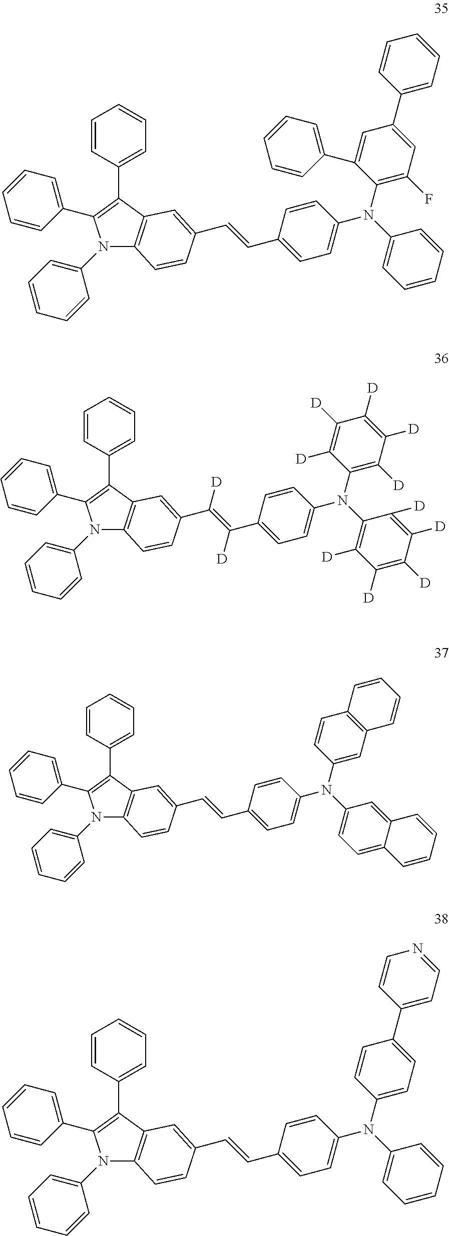 [SMILES: Fc1cc(-c2ccccc2)cc(-c2ccccc2)c1N(c1ccccc1)c1ccc(/C=C/c2ccc3c(c2)c(-c2ccccc2)c(-c2ccccc2)n3-c2ccccc2)cc1 ; [2H]/C(=C(/[2H])c1ccc2c(c1)c(-c1ccccc1)c(-c1ccccc1)n2-c1ccccc1)c1ccc(N(c2c([2H])c([2H])c([2H])c([2H])c2[2H])c2c([2H])c([2H])c([2H])c([2H])c2[2H])cc1 ; C(=C\c1ccc2c(c1)c(-c1ccccc1)c(-c1ccccc1)n2-c1ccccc1)/c1ccc(N(c2ccc3ccccc3c2)c2ccc3ccccc3c2)cc1 ; C(=C\c1ccc2c(c1)c(-c1ccccc1)c(-c1ccccc1)n2-c1ccccc1)/c1ccc(N(c2ccccc2)c2ccc(-c3ccncc3)cc2)cc1]

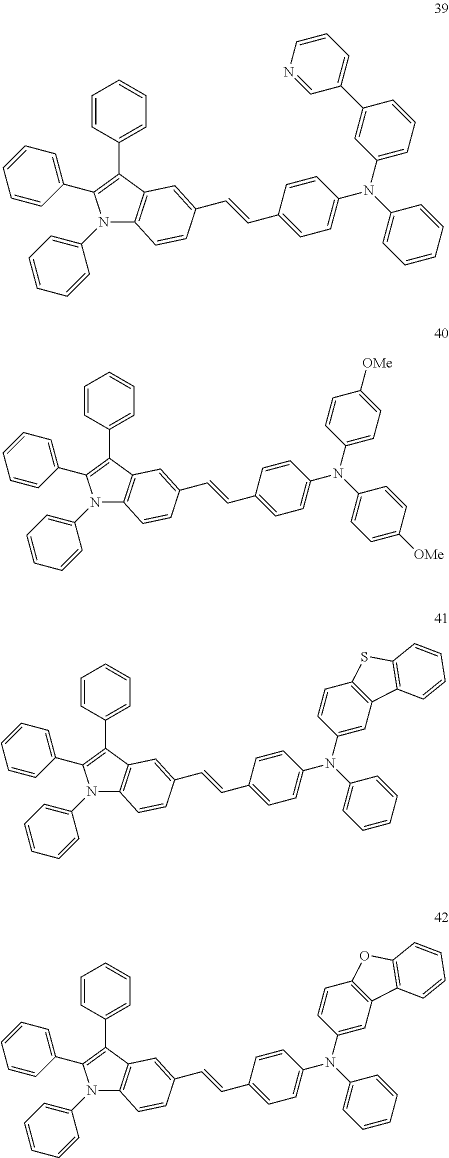 [SMILES: C(=C\c1ccc2c(c1)c(-c1ccccc1)c(-c1ccccc1)n2-c1ccccc1)/c1ccc(N(c2ccccc2)c2cccc(-c3cccnc3)c2)cc1 ; COc1ccc(N(c2ccc(/C=C/c3ccc4c(c3)c(-c3ccccc3)c(-c3ccccc3)n4-c3ccccc3)cc2)c2ccc(OC)cc2)cc1 ; C(=C\c1ccc2c(c1)c(-c1ccccc1)c(-c1ccccc1)n2-c1ccccc1)/c1ccc(N(c2ccccc2)c2ccc3sc4ccccc4c3c2)cc1 ; C(=C\c1ccc2c(c1)c(-c1ccccc1)c(-c1ccccc1)n2-c1ccccc1)/c1ccc(N(c2ccccc2)c2ccc3oc4ccccc4c3c2)cc1]

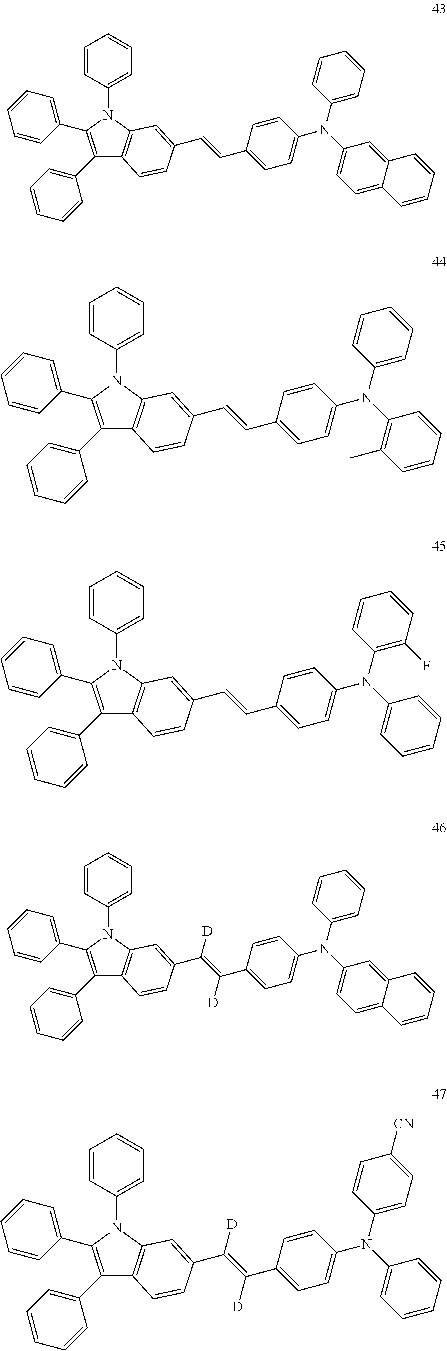 [SMILES: C(=C\c1ccc2c(-c3ccccc3)c(-c3ccccc3)n(-c3ccccc3)c2c1)/c1ccc(N(c2ccccc2)c2ccc3ccccc3c2)cc1 ; Cc1ccccc1N(c1ccccc1)c1ccc(/C=C/c2ccc3c(-c4ccccc4)c(-c4ccccc4)n(-c4ccccc4)c3c2)cc1 ; Fc1ccccc1N(c1ccccc1)c1ccc(/C=C/c2ccc3c(-c4ccccc4)c(-c4ccccc4)n(-c4ccccc4)c3c2)cc1 ; [2H]/C(=C(/[2H])c1ccc2c(-c3ccccc3)c(-c3ccccc3)n(-c3ccccc3)c2c1)c1ccc(N(c2ccccc2)c2ccc3ccccc3c2)cc1 ; [2H]/C(=C(/[2H])c1ccc2c(-c3ccccc3)c(-c3ccccc3)n(-c3ccccc3)c2c1)c1ccc(N(c2ccccc2)c2ccc(C#N)cc2)cc1]

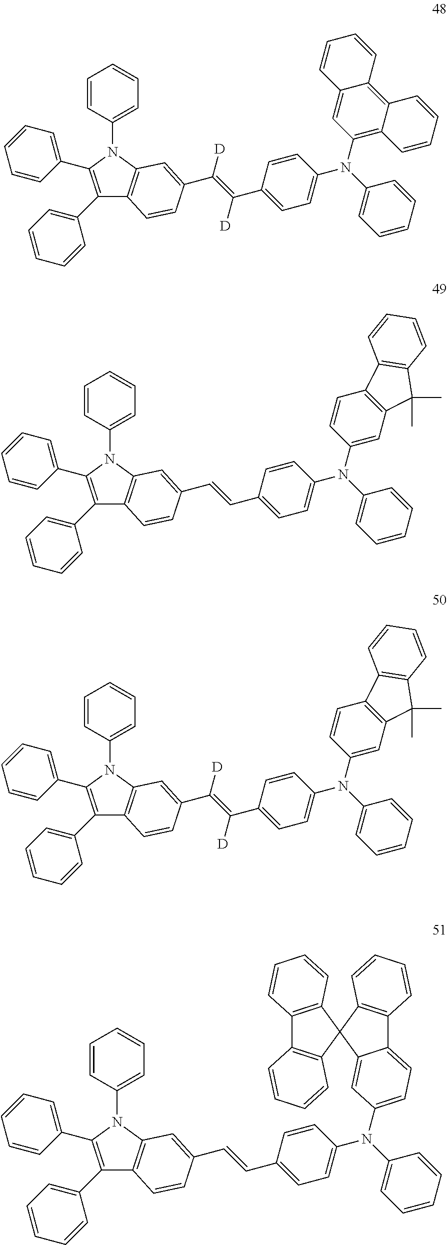 [SMILES: [2H]/C(=C(/[2H])c1ccc2c(-c3ccccc3)c(-c3ccccc3)n(-c3ccccc3)c2c1)c1ccc(N(c2ccccc2)c2cc3ccccc3c3ccccc23)cc1 ; CC1(C)c2ccccc2-c2ccc(N(c3ccccc3)c3ccc(/C=C/c4ccc5c(-c6ccccc6)c(-c6ccccc6)n(-c6ccccc6)c5c4)cc3)cc21 ; [2H]/C(=C(/[2H])c1ccc2c(-c3ccccc3)c(-c3ccccc3)n(-c3ccccc3)c2c1)c1ccc(N(c2ccccc2)c2ccc3c(c2)C(C)(C)c2ccccc2-3)cc1 ; C(=C\c1ccc2c(-c3ccccc3)c(-c3ccccc3)n(-c3ccccc3)c2c1)/c1ccc(N(c2ccccc2)c2ccc3c(c2)C2(c4ccccc4-c4ccccc42)c2ccccc2-3)cc1]

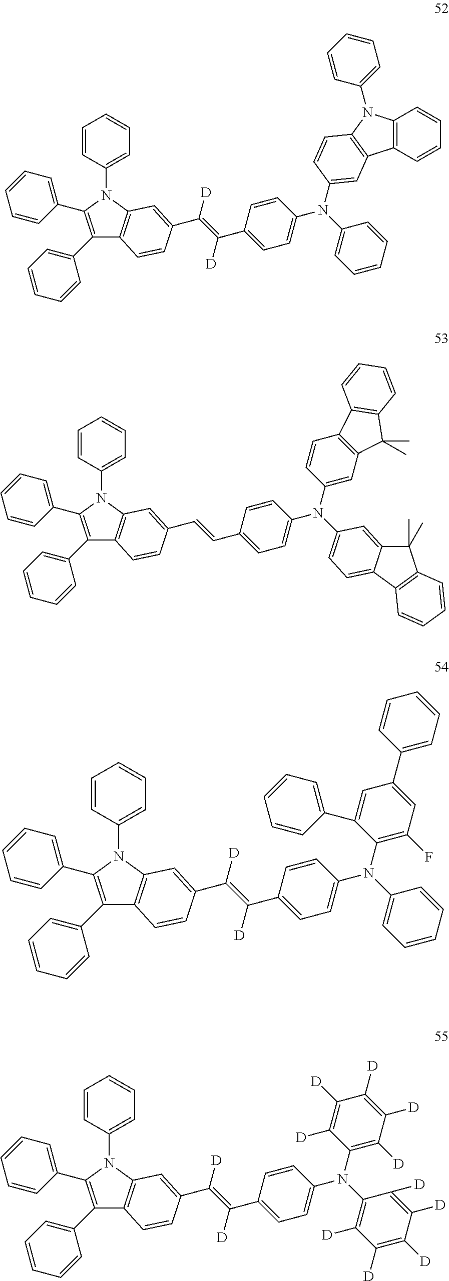

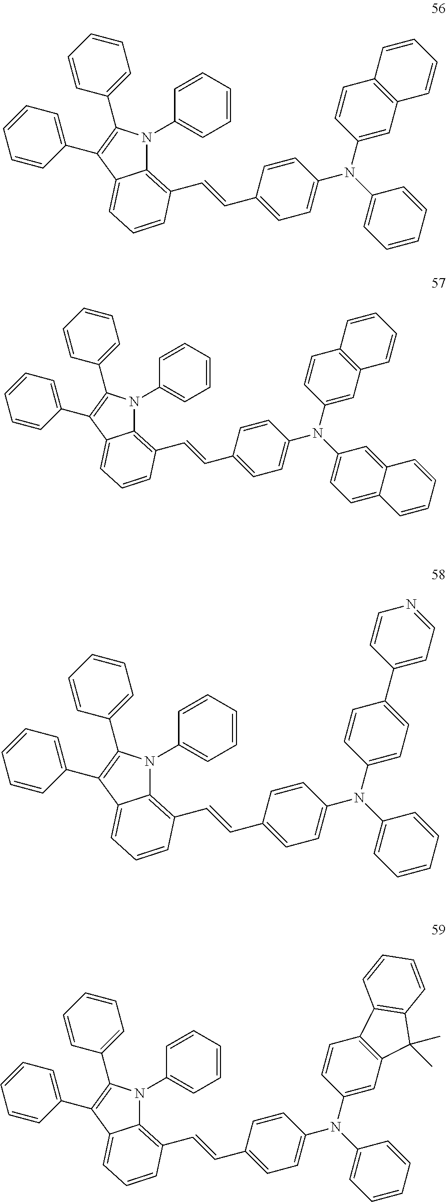 [SMILES: C(=C\c1cccc2c(-c3ccccc3)c(-c3ccccc3)n(-c3ccccc3)c12)/c1ccc(N(c2ccccc2)c2ccc3ccccc3c2)cc1 ; C(=C\c1cccc2c(-c3ccccc3)c(-c3ccccc3)n(-c3ccccc3)c12)/c1ccc(N(c2ccc3ccccc3c2)c2ccc3ccccc3c2)cc1 ; C(=C\c1cccc2c(-c3ccccc3)c(-c3ccccc3)n(-c3ccccc3)c12)/c1ccc(N(c2ccccc2)c2ccc(-c3ccncc3)cc2)cc1 ; CC1(C)c2ccccc2-c2ccc(N(c3ccccc3)c3ccc(/C=C/c4cccc5c(-c6ccccc6)c(-c6ccccc6)n(-c6ccccc6)c45)cc3)cc21]

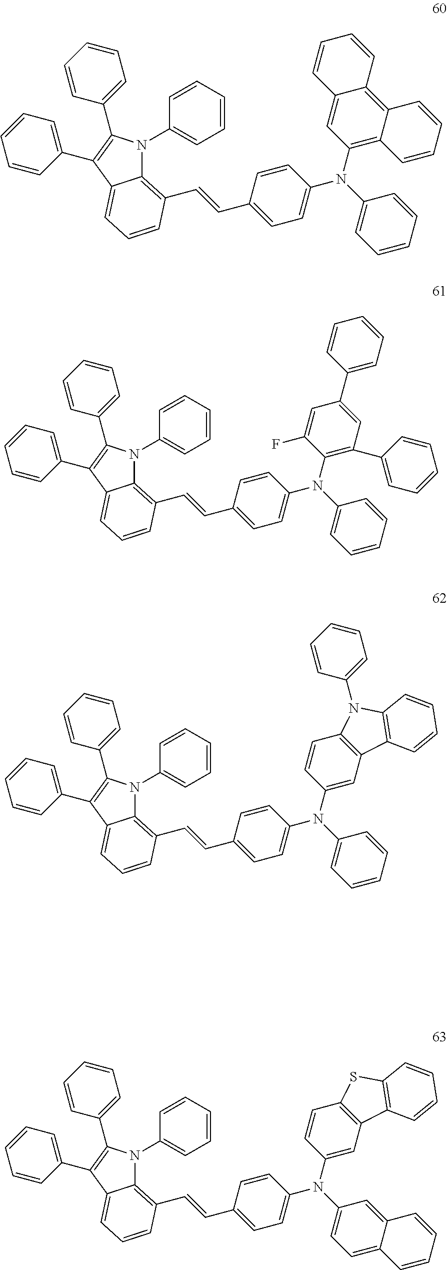 [SMILES: C(=C\c1cccc2c(-c3ccccc3)c(-c3ccccc3)n(-c3ccccc3)c12)/c1ccc(N(c2ccccc2)c2cc3ccccc3c3ccccc23)cc1 ; Fc1cc(-c2ccccc2)cc(-c2ccccc2)c1N(c1ccccc1)c1ccc(/C=C/c2cccc3c(-c4ccccc4)c(-c4ccccc4)n(-c4ccccc4)c23)cc1 ; C(=C\c1cccc2c(-c3ccccc3)c(-c3ccccc3)n(-c3ccccc3)c12)/c1ccc(N(c2ccccc2)c2ccc3c(c2)c2ccccc2n3-c2ccccc2)cc1 ; C(=C\c1cccc2c(-c3ccccc3)c(-c3ccccc3)n(-c3ccccc3)c12)/c1ccc(N(c2ccc3ccccc3c2)c2ccc3sc4ccccc4c3c2)cc1]

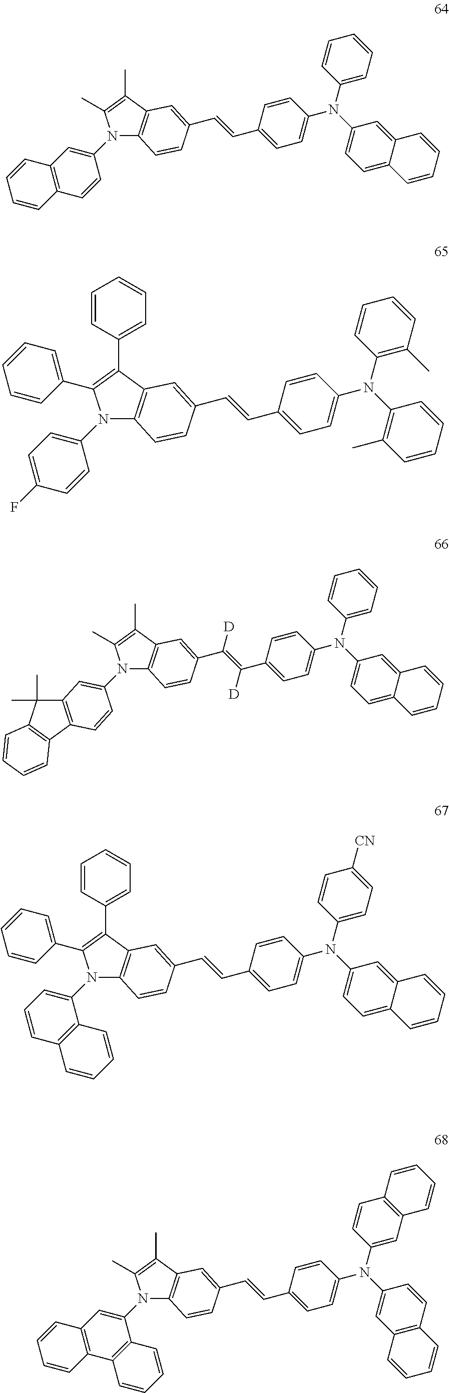 [SMILES: Cc1c(C)n(-c2ccc3ccccc3c2)c2ccc(/C=C/c3ccc(N(c4ccccc4)c4ccc5ccccc5c4)cc3)cc12 ; Cc1ccccc1N(c1ccc(/C=C/c2ccc3c(c2)c(-c2ccccc2)c(-c2ccccc2)n3-c2ccc(F)cc2)cc1)c1ccccc1C ; [2H]/C(=C(/[2H])c1ccc2c(c1)c(C)c(C)n2-c1ccc2c(c1)C(C)(C)c1ccccc1-2)c1ccc(N(c2ccccc2)c2ccc3ccccc3c2)cc1 ; N#Cc1ccc(N(c2ccc(/C=C/c3ccc4c(c3)c(-c3ccccc3)c(-c3ccccc3)n4-c3cccc4ccccc34)cc2)c2ccc3ccccc3c2)cc1 ; Cc1c(C)n(-c2cc3ccccc3c3ccccc23)c2ccc(/C=C/c3ccc(N(c4ccc5ccccc5c4)c4ccc5ccccc5c4)cc3)cc12]

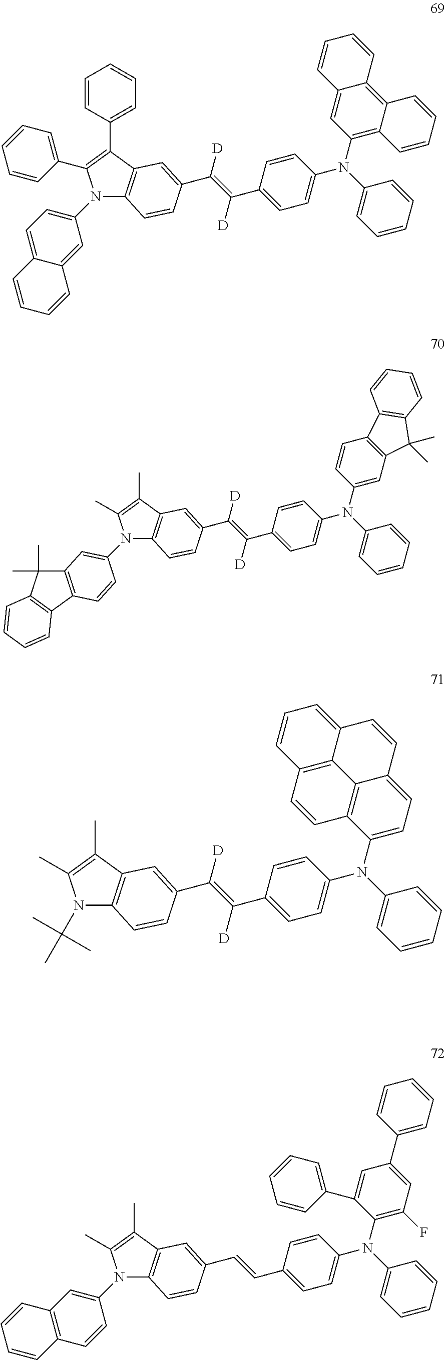

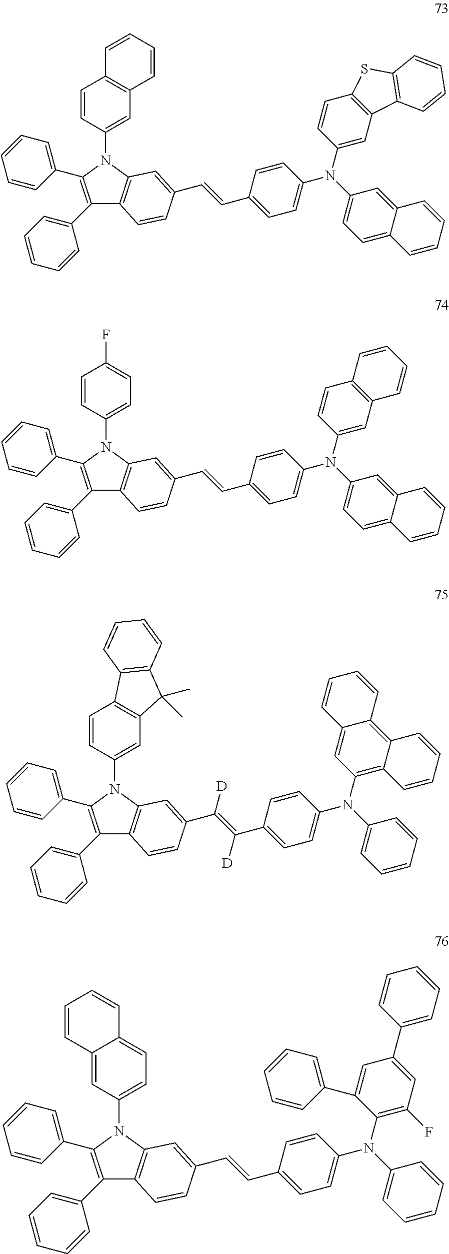 [SMILES: C(=C\c1ccc2c(-c3ccccc3)c(-c3ccccc3)n(-c3ccc4ccccc4c3)c2c1)/c1ccc(N(c2ccc3ccccc3c2)c2ccc3sc4ccccc4c3c2)cc1 ; Fc1ccc(-n2c(-c3ccccc3)c(-c3ccccc3)c3ccc(/C=C/c4ccc(N(c5ccc6ccccc6c5)c5ccc6ccccc6c5)cc4)cc32)cc1 ; [2H]/C(=C(/[2H])c1ccc2c(-c3ccccc3)c(-c3ccccc3)n(-c3ccc4c(c3)C(C)(C)c3ccccc3-4)c2c1)c1ccc(N(c2ccccc2)c2cc3ccccc3c3ccccc23)cc1 ; Fc1cc(-c2ccccc2)cc(-c2ccccc2)c1N(c1ccccc1)c1ccc(/C=C/c2ccc3c(-c4ccccc4)c(-c4ccccc4)n(-c4ccc5ccccc5c4)c3c2)cc1]

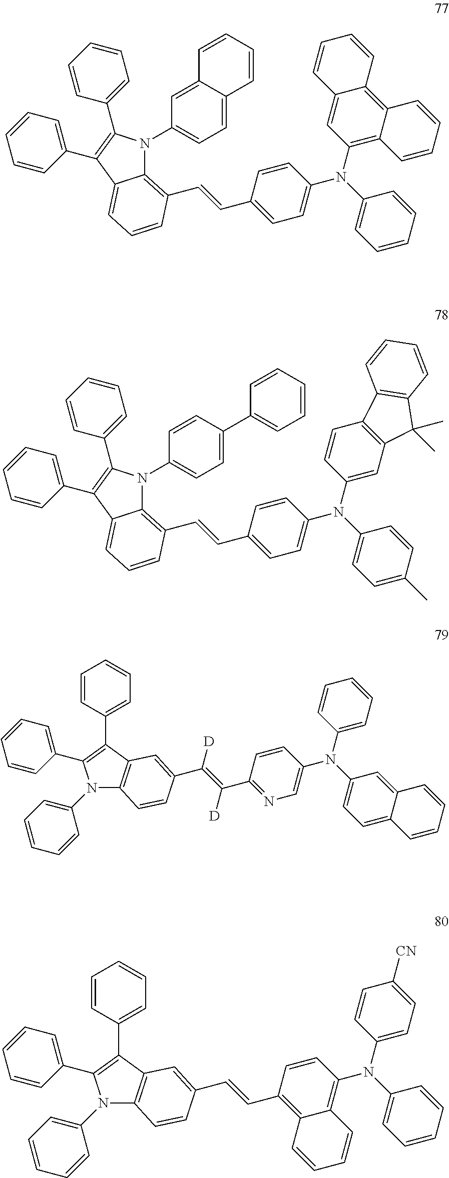 [SMILES: C(=C\c1cccc2c(-c3ccccc3)c(-c3ccccc3)n(-c3ccc4ccccc4c3)c12)/c1ccc(N(c2ccccc2)c2cc3ccccc3c3ccccc23)cc1 ; Cc1ccc(N(c2ccc(/C=C/c3cccc4c(-c5ccccc5)c(-c5ccccc5)n(-c5ccc(-c6ccccc6)cc5)c34)cc2)c2ccc3c(c2)C(C)(C)c2ccccc2-3)cc1 ; [2H]/C(=C(/[2H])c1ccc(N(c2ccccc2)c2ccc3ccccc3c2)cn1)c1ccc2c(c1)c(-c1ccccc1)c(-c1ccccc1)n2-c1ccccc1 ; N#Cc1ccc(N(c2ccccc2)c2ccc(/C=C/c3ccc4c(c3)c(-c3ccccc3)c(-c3ccccc3)n4-c3ccccc3)c3ccccc23)cc1]

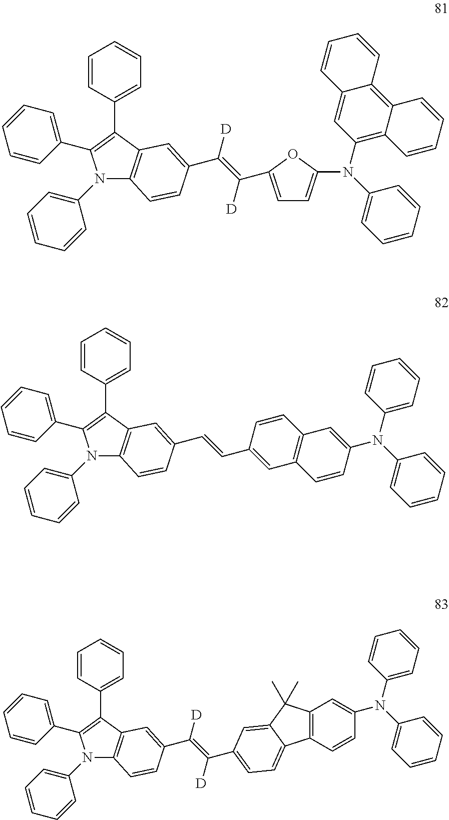 [SMILES: [2H]/C(=C(/[2H])c1ccc(N(c2ccccc2)c2cc3ccccc3c3ccccc23)o1)c1ccc2c(c1)c(-c1ccccc1)c(-c1ccccc1)n2-c1ccccc1 ; C(=C\c1ccc2c(c1)c(-c1ccccc1)c(-c1ccccc1)n2-c1ccccc1)/c1ccc2cc(N(c3ccccc3)c3ccccc3)ccc2c1 ; [2H]/C(=C(/[2H])c1ccc2c(c1)c(-c1ccccc1)c(-c1ccccc1)n2-c1ccccc1)c1ccc2c(c1)C(C)(C)c1cc(N(c3ccccc3)c3ccccc3)ccc1-2]

The heterocyclic compound represented by Formula 1A may function as a light-emission material, a hole injection material, and/or a hole transport material which are used in an organic light-emitting diode. The heterocyclic compound represented by Formula 1 having a heteroring in its molecular structure may have a high glass transition temperature (Tg) or melting point due to the introduction of the heteroring therein. Accordingly, during light-emission, an organic light-emitting diode including the heterocyclic compound represented by Formula 1A has a resistance against a Joule's heat generated in an organic layer during light emission, between organic layers, or between an organic layer and a metallic electrode, and thus has a stronger resistance under high-temperature environments. Also, if a substituent, such as a fluorine group, is introduced to the heterocyclic compound represented by Formula 1A, the morphology of an organic layer is improved and thus an organic light-emitting diode having the organic layer may have improved characteristics. Also, the heterocyclic compound represented by Formula 1A has only one aryl amino group therein and thus, a deep blue color may be embodied and thereby, color purity may be improved.

The term “substituted A” in the term “substituted or unsubstituted A (where A is an arbitrary substituent)” used herein refers to “a case in which one or more hydrogen atoms of the A are substituted with a deuterium atom, a halogen atom, a hydroxyl group, a cyano group, a nitro group, an amino group, a hydrazine group, a hydrazone group, a carboxyl group or a salt derivative thereof, a sulfonic acid group or a salt derivative thereof, a phosphoric acid group or a salt derivative thereof, C₁-C₃₀ alkyl group, C₂-C₃₀ alkenyl group, C₂-C₃₀ alkynyl group, C₁-C₃₀ alkoxy group, C₃-C₃₀ cycloalkyl group, C₃-C₃₀ cycloalkenyl group, C₅-C₃₀ aryl group, C₅-C₃₀ aryloxy group, C₅-C₃₀ arylthio group, C₃-C₃₀ heteroaryl group, a group represented by N(Q₁₀₁)(Q₁₀₂), or a group represented by Si(Q₁₀₃)(Q₁₀₄)(Q₁₀₅).” In this regard, Q₁₀₁ to Q₁₀₅ are each independently a hydrogen atom, a deuterium atom, a halogen atom, a hydroxyl group, a cyano group, an amino group, a nitro group, a carboxyl group, a C₁-C₃₀ alkyl group, a C₂-C₃₀ alkenyl group, a C₂-C₃₀ alkynyl group, a C₁-C₃₀ alkoxy group, a C₃-C₃₀ cycloalkyl group, a C₃-C₃₀ cycloalkenyl group, a C₅-C₃₀ aryl group, a C₅-C₃₀ aryloxy group, a C₅-C₃₀ arylthio group, or a C₃-C₃₀ heteroaryl group.

For example, the term “substituted A” refers to “a case in which one or more hydrogen atoms of the A are substituted with a deuterium atom, a halogen atom, a hydroxyl group, a cyano group, a nitro group, a carboxyl group, a methyl group, an ethyl group, a propyl group, a butyl group, a pentyl group, a methoxy group, an ethoxy group, a phenyl group, a biphenyl group, a pentalenyl group, a indenyl group, a naphthyl group, a azulenyl group, a heptalenyl group, an indacenyl group, an acenaphthyl group, a fluorenyl group, a spiro-fluorenyl group, a phenalenyl group, a phenanthrenyl group, a phenanthridinyl group, a phenanthrolinyl group, an anthryl group, a fluoranthenyl group, a triphenylenyl group, a pyrenyl group, a chrysenyl group, a naphthacenyl group, a picenyl group, a perylenyl group, a pentaphenyl group, a hexacenyl group, a pyrrolyl group, a imidazolyl group, a benzoimidazolyl group, a pyrazolyl group, a pyridinyl group, a pyrazinyl group, a pyrimidinyl group, a imidazopyrimidinyl group, a pyridazinyl group, a indolyl group, an isoindolyl group, a pyridoindolyl group, an indazolyl group, a purinyl group, a quinolinyl group, a benzoquinolinyl group, a phthalazinyl group, a naphthyridinyl group, a quinoxalinyl group, a quinazolinyl group, a carbazolyl group, a phenazinyl group, a furanyl group, a benzofuranyl group, a dibenzofuranyl group, a thiophenyl group, a benzothiophenyl group, a dibenzothiophenyl group, a thiazolyl group, an isothiazolyl group, a benzothiazolyl group, an oxazolyl group, a benzoxazolyl group, an isoxazolyl group, an oxadiazolyl group, a triazolyl group, a triazinyl group, a tetrazolyl group, a group represented by N(Q₁₀₁)(Q₁₀₂), or a group represented by Si(Q₁₀₃)(Q₁₀₄)(Q₁₀₅).

The unsubstituted C₁-C₃₀ alkyl group used herein refers to a linear or branched saturated hydrocarbonyl group of alkane from which one hydrogen atom is deficient. Examples of the unsubstituted C₁-C₃₀ alkyl group are a methyl group, an ethyl group, a propyl group, an isobutyl group, a sec-butyl group, a pentyl group, an iso-amyl group, a hexyl group, etc. A substituent of the substituted C₁-C₃₀ alkyl group may be any one of the substituents presented above where the term “substituted A” is described in detail.

The unsubstituted C₂-C₃₀ alkenyl group used herein refers to a terminal group having at least one carbon-carbon double blond at the center or at a terminal of the substituted and unsubstituted C₂-C₃₀ alkyl group. Examples of the unsubstituted C₂-C₃₀ alkenyl group are an ethenyl group, a propenyl group, a butenyl group, a pentenyl group, a hexenyl group, a heptenyl group, an octenyl group, a propadienyl group, an isoprenyl group, and an allyl group. A substituent of the substituted C₂-C₃₀ alkenyl group may be any one of the substituents presented above where the term “substituted A” is described in detail.

The unsubstituted C₂-C₃₀ alkynyl group used herein refers to a terminal group having at least one carbon-carbon triple bond at the center or at a terminal of the substituted and unsubstituted C₂-C₃₀ alkyl group. Examples of the unsubstituted C₂-C₃₀ alkynyl group are an ethynyl group, a propynyl group, an acetylenyl group, etc. A substituent of the substituted C₂-C₃₀ alkynyl group may be any one of the substituents presented above where the term “substituted A” is described in detail.

The unsubstituted C₁-C₃₀ alkoxy group used herein has a formula represented by —OY where Y is the unsubstituted C₁-C₃₀ alkyl group as defined above. Examples of the unsubstituted C₁-C₃₀ alkoxy group are a methoxy group, an ethoxy group, an isopropyloxy group, a butoxy group, a pentoxy group, etc. A substituent of the substituted C₁-C₃₀ alkoxy group may be any one of the substituents presented above where the term “substituted A” is described in detail.

The unsubstituted C₃-C₃₀ cycloalkyl group used herein refers to a cyclic saturated hydrocarbonyl group. Examples of the unsubstituted C₃-C₃₀ cycloalkyl group are a cyclopropyl group, a cyclobutyl group, a cyclopentyl group, a cyclohexyl group, a cyclooctyl group, etc. A substituent of the substituted C₃-C₃₀ cycloalkyl group may be any one of the substituents presented above where the term “substituted A” is described in detail.

The unsubstituted C₃-C₃₀ cycloalkenyl group used herein refers to a cyclic unsaturated hydrocarbonyl group having one or more carbon double bonds that are not an aromatic ring. Examples of the unsubstituted C₃-C₃₀ cycloalkenyl group are a cyclopropenyl group, a cyclobutenyl group, a cyclopentenyl group, a cyclohexenyl group, a cycloheptenyl group, a 1,3-cyclohexadienyl group, a 1,4-cyclohexadienyl group, a 2,4-cycloheptadienyl group, a 1,5-cyclooctadienyl group, etc. A substituent of the substituted C₃-C₃₀ cycloalkenyl group may be any one of the substituents presented above where the term “substituted A” is described in detail.

The unsubstituted C₅-C₃₀ aryl group used herein refers to a monovalent group having a carbocyclic aromatic system in which the number of carbon atoms is 5 to 30, and may be a monocyclic group or a polycyclic group. If the unsubstituted C₅-C₃₀ aryl group is a polycyclic group, two or more rings contained in the unsubstituted C₅-C₃₀ aryl group may be fused. Examples of the unsubstituted C₅-C₃₀ aryl group are a phenyl group, a pentalenyl group, an indenyl group, a naphthyl group, an azulenyl group, a heptalenyl group, an indacenyl group, an acenaphthyl group, a fluorenyl group, a spiro-fluorenyl group, a phenalenyl group, a phenanthrenyl group, an anthryl group, a fluoranthenyl group, a triphenylenyl group, a pyrenyl group, a chrycenyl group, a naphthacenyl group, a picenyl group, a perylenyl group, a pentaphenyl group, and a hexacenyl. A substituent of the substituted C₅-C₃₀ aryl group may be any one of the substituents presented above where the term “substituted A” is described in detail.

The unsubstituted C₅-C₃₀ aryloxy group used herein refers to a monovalent group wherein a carbon atom of the C₅-C₃₀ aryl group is attached via an oxygen linker (—O—). A substituent of the substituted C₅-C₃₀ aryloxy group may be any one of the substituents presented above where the term “substituted A” is described in detail.

The unsubstituted C₅-C₃₀ arylthio group used herein refers to a monovalent group wherein a carbon atom of the C₅-C₃₀ aryl group is attached via a sulfur linker (—S—). Examples of the unsubstituted C₅-C₃₀ arylthio group are a phenyl thio group, a naphthyl thio group, an indanylthio group, and an indenyl thio group. A substituent of the substituted C₅-C₃₀ arylthio group may be any one of the substituents presented above where the term “substituted A” is described in detail.

The unsubstituted C₃-C₃₀ heteroaryl group used herein refers to a monovalent group that has at least one ring having one or more heteroatoms selected from the group consisting of nitrogen (N), oxygen (O), phosphorous (P), and sulfur (S) and may be a monocyclic or polycyclic group. If the unsubstituted C₃-C₃₀ heteroaryl group is a polycyclic group, two or more rings contained in the unsubstituted C₃-C₃₀ heteroaryl group may be fused. Examples of the unsubstituted C₃-C₃₀ heteroaryl group are a pyrrolyl group, an imidazolyl group, a pyrazolyl group, a pyridinyl group, a pyrazinyl group, a pyrimidinyl group, a pyridazinyl group, an isoindolyl group, an indolyl group, an indazolyl group, a purinyl group, a quinolinyl group, a benzoquinolinyl group, a phthalazinyl group, a naphthyridinyl group, a quinoxalinyl group, a quinazolinyl group, a cinnolinyl group, a carbazolyl group, phenanthridinyl group, an acridinyl group, a phenanthrolinyl group, a phenazinyl group, a benzooxazolyl group, a benzoimidazolyl group, a furanyl group, a benzofuranyl group, a thiophenyl group, a benzothiophenyl group, thiazolyl group, an isothiazolyl group, a benzothiazolyl group, an isoxazolyl group, an oxazolyl group, a triazolyl group, a tetrazolyl group, an oxadiazolyl group, a triazinyl group, a benzooxazolyl group, etc. A substituent of the substituted C₃-C₃₀ heteroaryl group may be any one of the substituents presented above where the term “substituted A” is described in detail.

The unsubstituted C₁-C₃₀ alkylene group used herein refers to a linear or branched divalent group of alkane from which two hydrogen atoms are deficient. Examples of the unsubstituted C₁-C₃₀ alkylene group may be understood by referring to the examples of the unsubstituted C₁-C₃₀ alkyl group presented above. A substituent of the substituted C₁-C₃₀ alkylene group may be any one of the substituents presented above where the term “substituted A” is described in detail.

The unsubstituted C₅-C₃₀ arylene group used herein refers to a divalent group having a carbocyclic aromatic system having 5 to 30 carbon atoms, and the divalent group may be a monocyclic or polycyclic group. Examples of the unsubstituted C₅-C₃₀ arylene may be understood by referring to the examples of the unsubstituted C₅-C₃₀ aryl group. A substituent of the substituted C₅-C₃₀ arylene group may be any one of the substituents presented above where the term “substituted A” is described in detail.

The heterocyclic compound represented by Formula 1 may be synthesized by using known organic synthesis methods. The Heterocyclic compound synthesis methods may be obvious to one of ordinary skill in the art with reference to examples, one of which will now be described in detail.

The heterocyclic compound represented by Formula 1A may be used in an organic light-emitting diode.

Another aspect of the present invention provides an organic light-emitting diode including a first electrode; a second electrode disposed facing the first electrode; and an organic layer interposed between the first electrode and the second electrode. The organic layer may include at least one layer and the organic layer may include one or more kinds of the heterocyclic compound represented by Formula 1A.

The term “organic layer” used herein refers to a layer that includes an organic compound and that is a single or multi-layer. For example, the organic layer may include at least one layer of a hole injection layer, a hole transport layer, a hole injection and transport layer having a hole injection capability and a hole transporting capability, an electron blocking layer, an emission layer, a hole blocking layer, an electron injection layer, an electron transport layer, and an electron injection and transport layer having an electron injection capability and an electron transporting capability. The organic layer may be formed of an organic compound only, and may further include an inorganic compound or an inorganic material. For example, the organic layer may include, in addition to an organic compound, an inorganic compound or inorganic material, for example, an organometallic complex within a single layer. Alternatively, the organic layer may include a layer including an organic compound and a layer including only an inorganic compound or an inorganic material.

The organic layer may include one or more kinds of the heterocyclic compounds in either a single layer or different layers. For example, the organic layer may include one kind of heterocyclic compound as a luminescent dopant in the emission layer and another kind of heterocyclic compound as a hole transport material in the hole transport layer. Alternatively, for example, the organic layer may include one kind of heterocyclic compound as a luminescent dopant and another kind of heterocyclic compound as a luminescent host in the emission layer. Alternatively, for example, the organic layer may include one kind of heterocyclic compound as a luminescent dopant and another kind of heterocyclic compound as a luminescent host in the emission layer, and another kind of heterocyclic compound as a hole transport material in the hole transport layer.

According to an embodiment of the present invention, the organic layer may include at least one of an emission layer, a hole injection layer, a hole transport layer, and a hole injection and transport layer having a hole injection capability and a hole transporting capability, wherein the at least one of an emission layer, a hole injection layer, a hole transport layer, and a hole injection and transport layer may include the heterocyclic compound.

For example, the organic light-emitting diode may include a structure of first electrode/hole injection layer/hole transport layer/emission layer/electron transport layer/electron injection layer/second electrode, wherein the emission layer may include the heterocyclic compound, the hole transport layer may include the heterocyclic compound, or the hole injection layer may include the heterocyclic compound. Alternatively, two or more layers selected from the emission layer, the hole transport layer, and the hole injection layer may include the heterocyclic compound. In this case, the respectively layers may include different heterocyclic compounds. As described above, 2 or more kinds of the heterocyclic compound may be used in a mixed form in the respective layers, and one kind of the heterocyclic compound may be used in a mixed form with other compounds.

According to an embodiment of the present invention, the organic layer may include an emission layer and the emission layer may include a host and a dopant, and the heterocyclic compound may be a fluorescent host, a phosphorescent host, or a fluorescent dopant of the emission layer.

According to an embodiment of the present invention, the organic layer may include an emission layer, and the emission layer may include an anthracene compound, an arylamine compound, or a styryl compound. In this regard, the emission layer may include or may not include the heterocyclic compound.

According to an embodiment of the present invention, the organic layer may include an emission layer, and the emission layer may include a host and a dopant, and the emission layer may further include a phosphorescent dopant. The phosphorescent dopant may be, for example, Ir, Pt, Os, Re, Ti, Zr, Hf, or an organometallic complex including a combination of two or more of these materials, but is not limited thereto. In this regard, the emission layer may include or may not include the heterocyclic compound.

At least one of the hole injection layer, the hole transport layer, and the hole injection and transport layer may further include, in addition to the heterocyclic compound, charge-generating material. The charge-generating material may be, for example, a p-dopant. In this regard, each of the hole injection layer, the hole transport layer, and the hole injection and transport layer may include or may not include the heterocyclic compound.

The organic layer may further include an electron transport layer, and the electron transport layer may further include an electron transport organic compound and a metal-containing material. The metal-containing material may include a Li complex. In this regard, the electron transport layer may include or may not include the heterocyclic compound.

At least one layer selected from the organic layers interposed between the first electrode and the second electrode may be formed by a deposition process or a wet process.

The term “wet process” used herein refers to a process in which a material is mixed with a solvent to prepare a mixture, and the mixture is provided on a substrate, followed by drying and/or heat treating so as to remove at least a portion of the solvent, thereby forming a film including the material on the substrate.

For example, the organic layer may be formed by using a typical vacuum deposition method. Alternatively, a mixture including the heterocyclic compound and a solvent may be provided on an organic layer formation region (for example, on an upper portion of a hole transport layer) by spin coating, spraying, ink-jet printing, dipping, casting, Gravia coating, bar coating, roll coating, wire bar coating, screen coating, flexo coating, offset coating, or laser transferring, and then, the mixture provided on the organic layer formation region is dried and/or heat treated to remove at least a portion of the solvent, thereby forming the organic layer.

Alternatively, after an organic layer is formed on a base film by using the wet process as described above, the organic layer may be transferred to an organic layer formation region (for example, an upper portion of the hole transport layer) by using, for example, a laser.

FIG. 1 is a schematic view of an organic light-emitting diode 10 according to an embodiment of the present invention. Hereinafter, with reference to FIG. 1, the structure of an organic light-emitting diode according to an embodiment of the present invention, and a method of manufacturing the organic light-emitting diode, according to an embodiment of the present invention, will be described in detail.

The organic light-emitting diode 10 sequentially includes a substrate 11, a first electrode 13, an organic layer 15, and a second electrode 17.

The substrate 11 may be any one of various substrates that are used in a known organic light-emitting device, and may be a glass substrate or a transparent plastic substrate with excellent mechanical strength, thermal stability, transparency, surface smoothness, ease of handling, and water repellency.

The first electrode 13 may be formed by providing a first electrode material on a substrate by deposition or sputtering. If the first electrode 13 is an anode, to allow holes to be injected thereinto easily, the first electrode material may be selected from materials having a high work function. Also, the first electrode 13 may be a reflection electrode or a transmission electrode. The first electrode material may be a transparent and highly conductive material group, such as an indium tin oxide (ITO), or an indium zinc oxide (IZO), tin oxide (SnO₂), zinc oxide (ZnO), etc. Alternatively, if magnesium (Mg), aluminum (Al), aluminum-lithium (Al—Li), calcium (Ca), magnesium-indium (Mg—In), magnesium-silver (Mg—Ag) etc, are used as the first electrode material group, the first electrode 13 may be formed as a reflection electrode.

The organic layer 15 is formed on the first electrode 13. As described above, the organic layer 15 indicates all layers interposed between the first electrode 13 and the second electrode 17, and the organic layer 15 may include a metallic complex. Herein, the organic layer 15 does not necessarily mean a layer that includes only an organic material.

The organic layer 15 may include a hole injection layer, a hole transport layer, an emission layer, an electron transport layer, and an electron injection layer.

The hole injection layer (HIL) may be formed on the first electrode 13 by using various methods, such as the vacuum deposition, spin-coating, casting, Langmuir-Blodgett (LB), or the like.

When a hole injection layer (HIL) is formed by vacuum deposition, the deposition conditions may vary according to a material that is used to form the HIL, and the structure and thermal characteristics of the HIL. For example, the deposition conditions may include a deposition temperature of about 100 to about 500° C., a vacuum pressure of about 10⁻⁸ to about 10⁻³ torr, and a deposition rate of about 0.01 to about 100 Å/sec. However, the deposition conditions are not limited thereto.

When the HIL is formed using spin coating as a wet process, coating conditions may vary according to the material used to form the HIL, and the structure and thermal properties of the HIL. For example, a coating speed may be from about 2,000 rpm to about 5,000 rpm, and a temperature at which a heat treatment is performed to remove a solvent after coating may be from about 80° C. to about 200° C. However, the coating conditions are not limited thereto.

A HIL material may be the heterocyclic compound of Formula 1A or a known hole injection material. Examples of a known hole injection material are a phthalocyanine compound, such as N,N′-diphenyl-N,N′-bis-[4-(phenyl-m-tolyl-amino)-phenyl]-biphenyl-4,4′-diamine (DNTPD), 4,4′,4″-tris(3-methylphenylphenylamino)triphenylamine (m-MTDATA), N,N′-di(1-naphthyl)-N,N′-diphenylbenzidine (NPB), 4,4′4″-tris(N,N-diphenylamino)triphenylamine (TDATA), 4,4′,4″-tris{N,-(2-naphthyl)-N-phenylamino}-triphenylamine (2T-NATA), polyaniline/dodecylbenzenesulfonic acid (PANI/DBSA), poly(3,4-ethylenedioxythiophene)/poly(4-styrenesulfonate) (PEDOT/PSS), polyaniline/camphor sulfonic acid (PANI/CSA), polyaniline/poly(4-styrenesulfonate) (PANI/PSS), etc. but is not limited thereto.

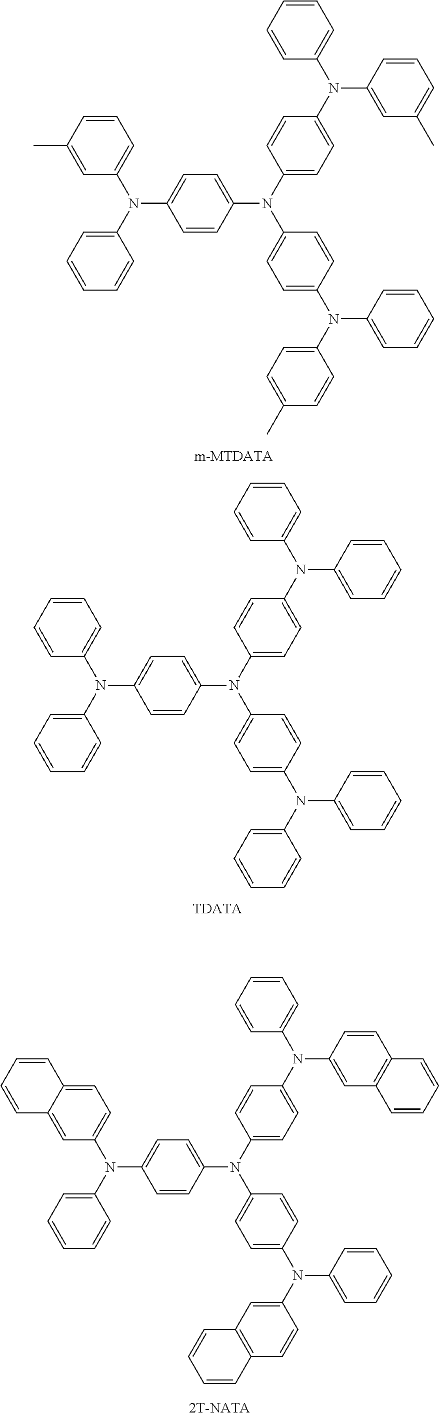 [SMILES: Cc1ccc(N(c2ccccc2)c2ccc(N(c3ccc(N(c4ccccc4)c4cccc(C)c4)cc3)c3ccc(N(c4ccccc4)c4cccc(C)c4)cc3)cc2)cc1 ; c1ccc(N(c2ccccc2)c2ccc(N(c3ccc(N(c4ccccc4)c4ccccc4)cc3)c3ccc(N(c4ccccc4)c4ccccc4)cc3)cc2)cc1 ; "TDATA" ; c1ccc(N(c2ccc(N(c3ccc(N(c4ccccc4)c4ccc5ccccc5c4)cc3)c3ccc(N(c4ccccc4)c4ccc5ccccc5c4)cc3)cc2)c2ccc3ccccc3c2)cc1]

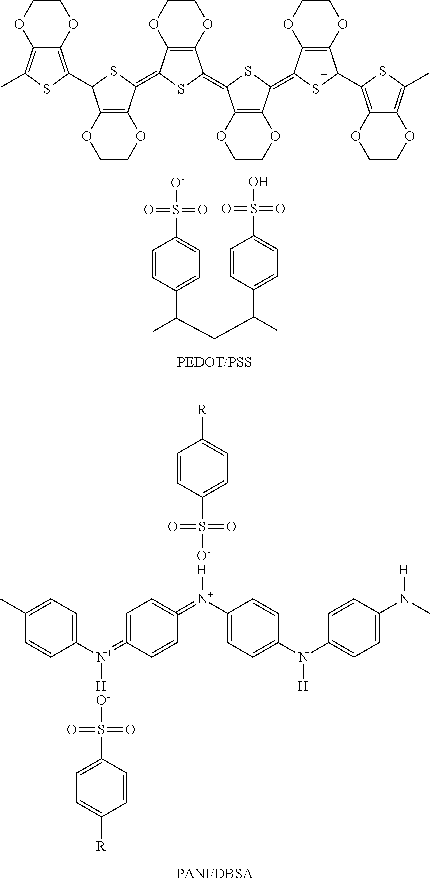

The HIL may have a thickness of about 100 Å to about 10,000 Å, for example, a thickness of about 100 Å to about 1,000 Å. When the thickness of the HIL is within these ranges, the HIL may have satisfactory hole injection characteristics without an increase in driving voltage.

Then, a hole transport layer (HTL) may be formed on the HIL by, various methods, such as the vacuum deposition, spin-coating, casting, LB, or the like. When the HTL is formed on the HIL by vacuum deposition or spin coating, the deposition or coating conditions may be similar to those applied to form the HIL, although the deposition or coating conditions may vary according to the material that is used to form the HTL.

A HTL material may be the heterocyclic compound of Formula 1A or a known hole transport material. Examples of a known hole transport material are a carbazole derivative, such as N-phenylcarbazole or polyvinylcarbazole; a triphenylamine-based material, such as N,N′-bis(3-methylphenyl)-N,N′-diphenyl-[1,1-biphenyl]-4,4′-diamine (TPD); an aromatic fused ring-containing amine derivative, such as 4,4′-bis[N-(1-naphthyl)-N-phenylamino]biphenyl (α-NPD), or 4,4′,4″-tris(N-carbazolyl)triphenylamine (TCTA), etc.

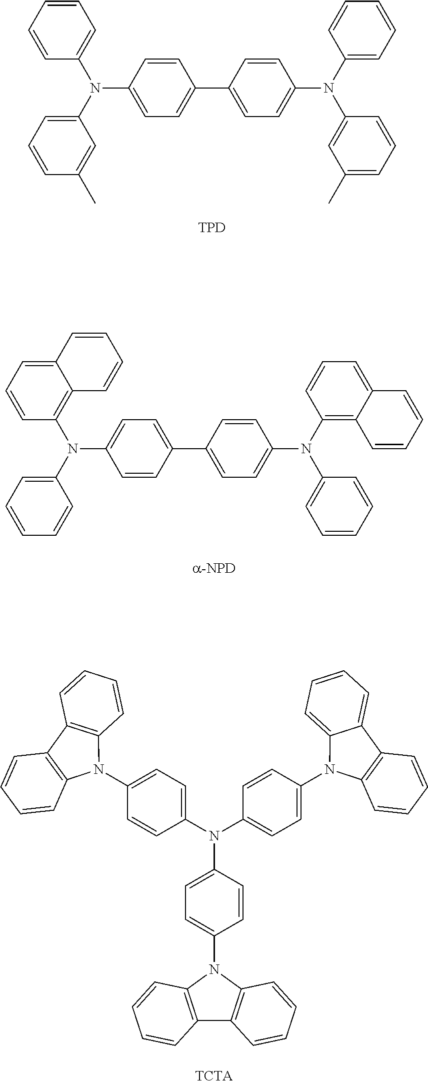 [SMILES: Cc1cccc(N(c2ccccc2)c2ccc(-c3ccc(N(c4ccccc4)c4cccc(C)c4)cc3)cc2)c1 ; "TPD" ; c1ccc(N(c2ccc(-c3ccc(N(c4ccccc4)c4cccc5ccccc45)cc3)cc2)c2cccc3ccccc23)cc1 ; c1ccc2c(c1)c1ccccc1n2-c1ccc(N(c2ccc(-n3c4ccccc4c4ccccc43)cc2)c2ccc(-n3c4ccccc4c4ccccc43)cc2)cc1]

The HTL may have a thickness of about 50 Å to about 1,000 Å, for example, a thickness of about 100 Å to about 800 Å. When the thickness of the HTL is within the above ranges, the HTL may have satisfactory hole transport characteristics without an increase in driving voltage.

Alternatively, instead of the HIL and the hole transport layer, a hole injection and transport layer having a hole injection capability and a hole transporting capability may be used. The hole injection and transport layer may be the heterocyclic compound of Formula 1A or a known material.

At least one of the HIL, the HTL, and the hole injection and transport layer may further include, in addition to a known hole injection material and a known hole transport material group, a charge-generating material for improving conductivity of a film.

The charge-generating material may be, for example, a p-dopant. Non-limiting examples of the p-dopant are a quinone derivative, such as tetracyanoquinodimethane (TCNQ) or 2,3,5,6-tetrafluoro-tetracyano-1,4-benzoquinonedimetein (F4TCNQ); a metal oxide, such as tungsten oxide or molybdenum oxide; a cyano group-containing compound, such as Compound 100 illustrated below, etc.

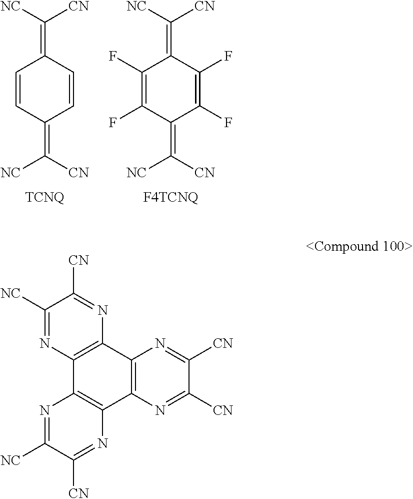

If the HIL, the hole transport layer, or the hole injection and transport layer having a hole injection capability and a hole transporting capability further includes the charge-generating material, the charge-generating material may be homogeneously or non-homogeneously dispersed in the layers.

An emission layer (EML) may be formed on the hole transport layer or the hole injection and transport layer having a hole injection capability and a hole transporting capability by, various methods, such as vacuum deposition, spin-coating, casting, LB, or the like. When the EML is formed by vacuum deposition or spin coating, the deposition or coating conditions may be similar to those applied to form the HIL, although the deposition or coating conditions may vary according to the material that is used to form the EML.

An EML material may be the heterocyclic compound of Formula 1A or one or more kinds of a known light-emission material (as a host and a dopant). If the EML includes the heterocyclic compound represented by Formula 1A, the EML may further include, in addition to the heterocyclic compound represented by Formula 1A, a known phosphorescent host, a known fluorescent host, a known phosphorescent dopant, or a known fluorescent dopant. The heterocyclic compound may function as a phosphorescent host, a fluorescent host, or a fluorescent dopant.

As a host, the heterocyclic compound represented by Formula 1A or a known host may be used. As a known host, for example, Alq₃, CBP(4,4′-N,N′-dicabazole-biphenyl), PVK(poly(n-vinylcabazole)), ADN(9,10-di(naphthalene-2-yl)anthracene), TCTA, TPBI(1,3,5-tris(N-phenylbenzimidazole-2-yl)benzene), TBADN(3-tert-butyl-9,10-di(naphth-2-yl)anthracene), DSA(distyrylarylene), E3, or the like may be used. However, the host may not be limited thereto.

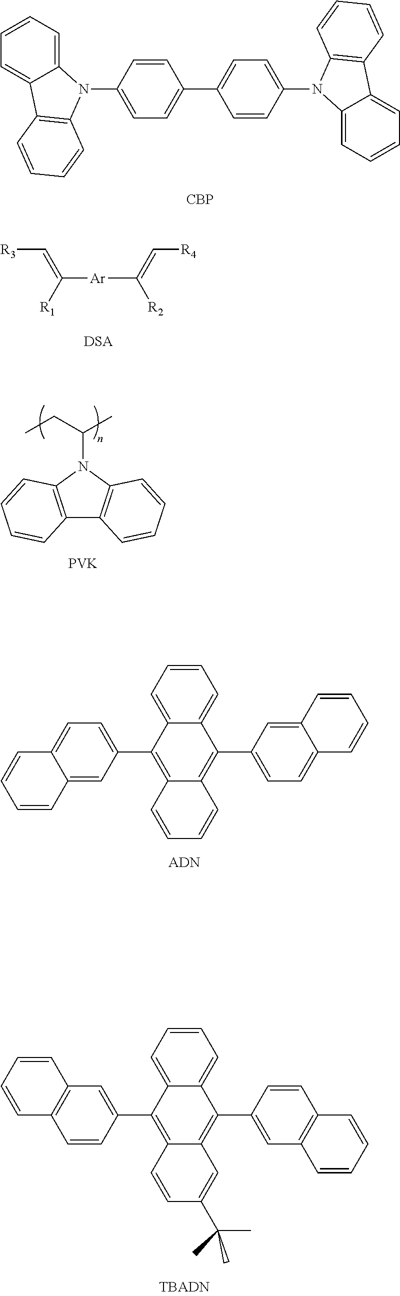

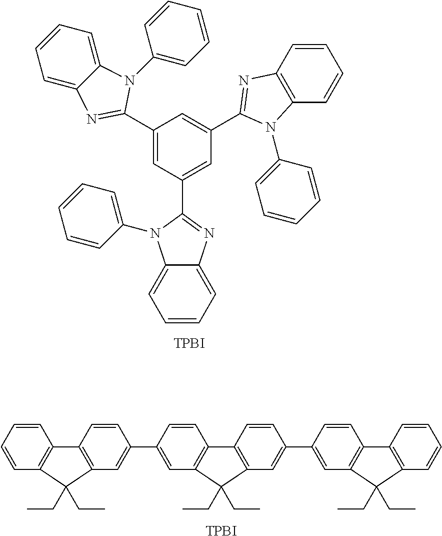

As the dopant, the heterocyclic compound represented by Formula 1A or a known dopant may be used. The dopant may be at least one of a fluorescent dopant and a phosphorescent dopant. The phosphorescent dopant may be Ir, Pt, Os, Re, Ti, Zr, Hf, or an organometallic complex including two or more combination of these materials, but are not limited thereto.

Also, as a known red dopant, Pt(II) Octaethylporphin (PtOEP), tris(2-phenylisoquinoline)iridium (Ir(piq)₃), bis(2-(2′-benzothienyl)-pyridinato-N,C3′)iridium(acetylacetonate) (Btp₂Ir(acac)), etc. may be used. However, other known red dopants may also be used herein.

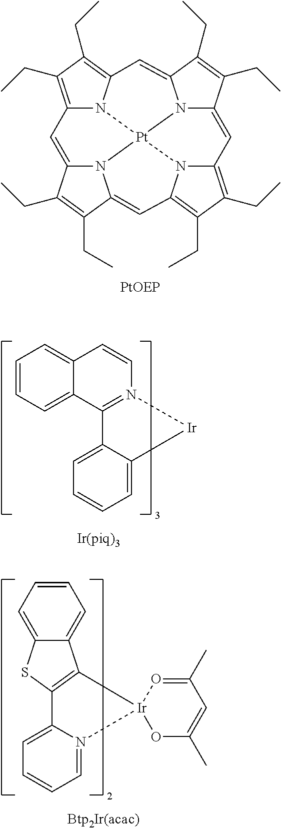

Also, as a known green dopant, tris(2-phenylpyridine) iridium (Ir(ppy)₃), bis(2-phenylpyridine)(Acetylacetonato)iridium(III) (Ir(ppy)₂(acac)), tris(2-(4-tolyl)phenylpiridine)iridium (Ir(mppy)₃), 10-(2-benzothiazolyl)-1,1,7,7-tetramethyl-2,3,6,7-tetrahydro-1H,5H,11H-[1]benzopyrano[6,7,8-ij]-quinolizin-11-one (C545T), etc. may be used. However, other known red dopants may also be used herein.

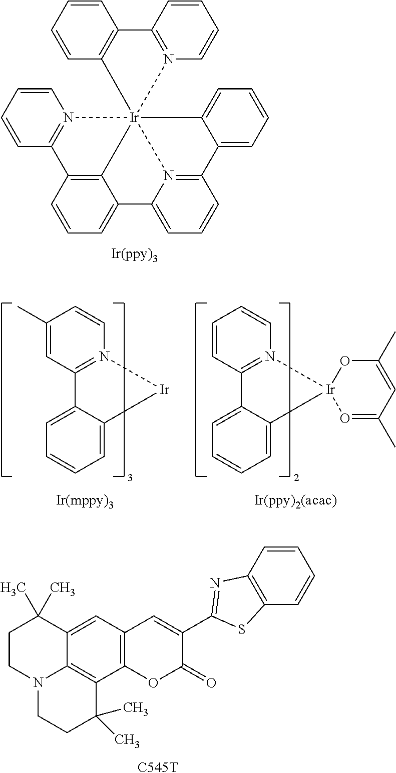

Also, as a known blue dopant, bis[3,5-difluoro-2-(2-pyridyl)phenyl](picolinato)iridium(III) (F₂Irpic), (F₂ppy)₂Ir(tmd), Ir(dfppz)₃, 4,4′-bis(2,2′-diphenylethen-1-yl)biphenyl (DPVBi), 4,4′-Bis[4-(diphenylamino)styryl]biphenyl (DPAVBi), 2,5,8,11-tetra-tert-butyl perylene (TBPe) etc. may be used. However, other known blue dopants may also be used herein.

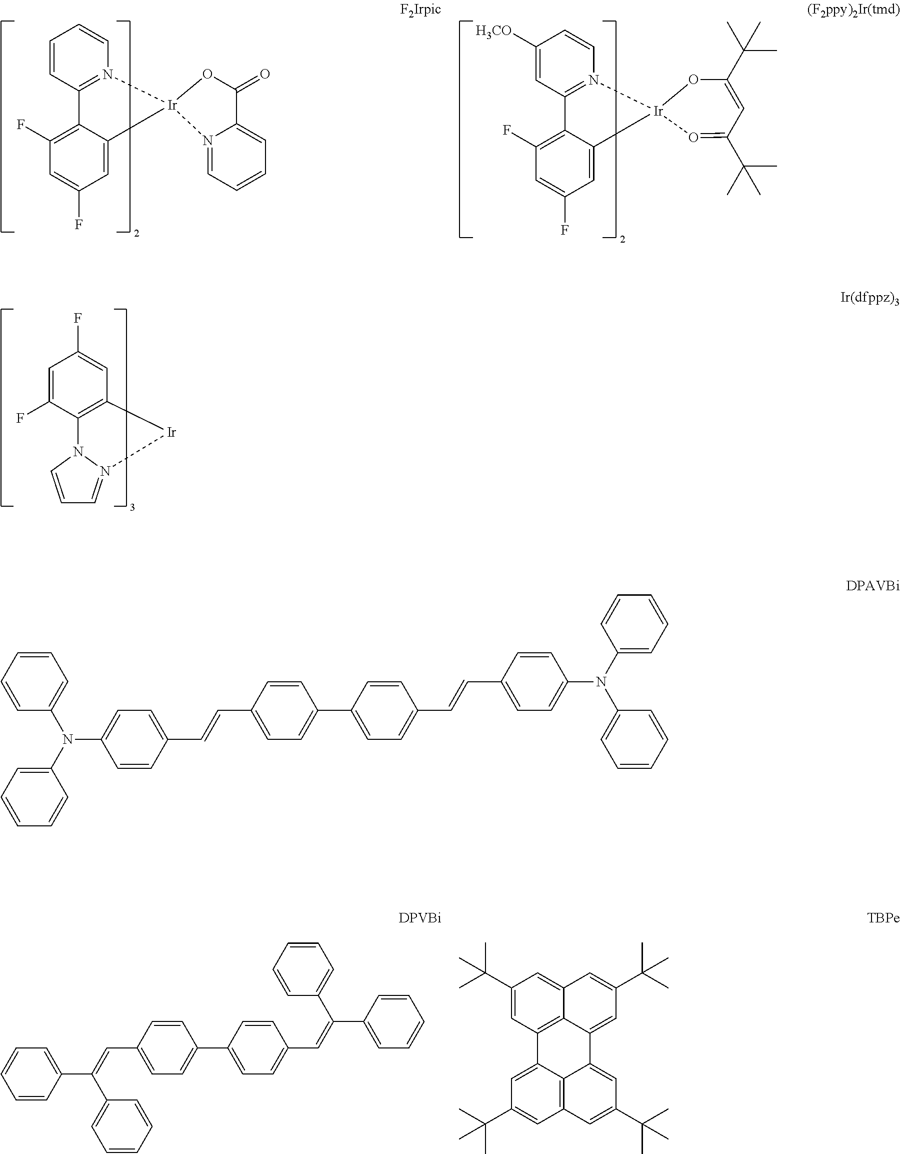

If the EML includes a host and a dopant, typically, an amount of the dopant may be in a range of about 0.01 to about 15 parts by weight based on host about 100 parts by weight of the host, but is not limited thereto.

A thickness of the EML may be in a range of about 100 Å to about 1,000 Å, for example, about 200 Å to about 600 Å. If the thickness of the EML is within the ranges described above, excellent luminescence characteristics may be obtained without a substantial increase in driving voltage.

If the EML includes a phosphorescent dopant, to prevent diffusion of a triple exciton or a hole into an electron transport layer, a hole blocking layer (HBL) may be formed between the electron transport layer and the EML by vacuum deposition, spin-coating, casting, LB, or the like. If the HBL is formed by vacuum deposition or spin coating, the deposition or coating conditions may be similar to those applied to form the HIL, although the deposition or coating conditions may vary according to the material that is used to form the HBL. As a HBL material, any one of known hole blocking materials may be used, and examples thereof are an oxadiazole derivative, a triazole derivative, a phenanthroline derivative, etc. For example, BCP may be used as a material for forming the HBL.

A thickness of the HBL may be from about 50 Å to about 1000 Å, for example, about 100 Å to about 300 Å. If the thickness of the HBL is within the ranges described above, excellent hole blocking characteristics may be obtained without a substantial increase in driving voltage.

Then, an electron transport layer (ETL) may be formed by using various methods, such as vacuum deposition, spin-coating, casting, LB, or the like. If the ETL is formed by vacuum deposition or spin coating, the deposition or coating conditions may be similar to those applied to form the HIL, although the deposition or coating conditions may vary according to the material that is used to form the ETL.

An ETL material may be a known electron transport material. Examples of a known electron transport material are a quinoline derivative, such as tris(8-quinolinolate)aluminum (Alq₃), 2,9-dimethyl-4,7-diphenyl-1,10-phenanthroline (BCP), 4,7-diphenyl-1,10-phenanthroline (Bphen), 3-(4-biphenylyl)-4-phenyl-5-tert-butylphenyl-1,2,4-triazole (TAZ), 4-(naphthalen-1-yl)-3,5-diphenyl-4H-1,2,4-triazole (NTAZ), 2-(4-biphenylyl)-5-(4-tert-butylphenyl)-1,3,4-oxadiazole (tBu-PBD), BAlq (a structure thereof is illustrated below), beryllium bis(benzoquinolin-10-olate (Bebq₂), 9,10-di(naphthalene-2-yl)anthracene (ADN), Compound 101, Compound 102, etc., but are not limited thereto.

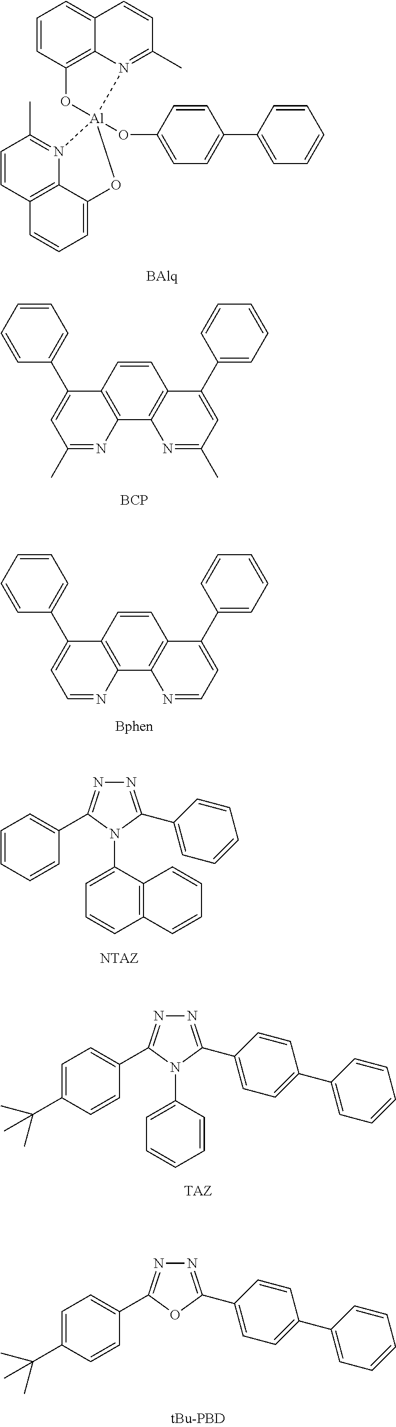

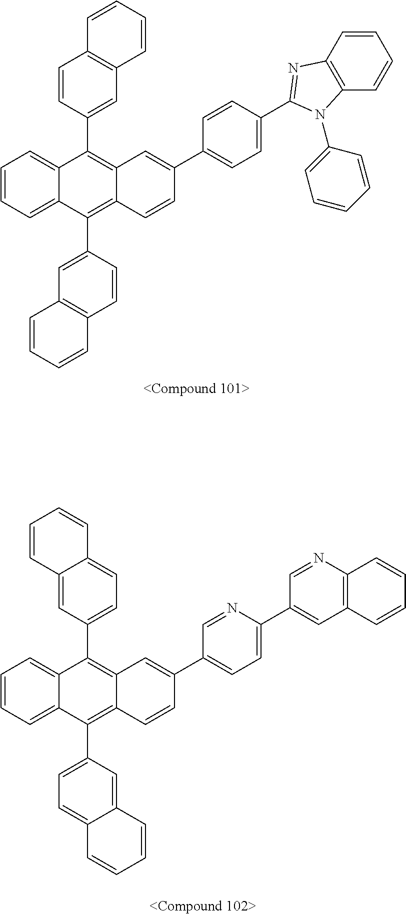 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc2cc(-c3c4ccccc4c(-c4ccc5ccccc5c4)c4cc(-c5ccc(-c6cnc7ccccc7c6)nc5)ccc34)ccc2c1]

A thickness of the ETL may be from about 100 Å to about 1,000 Å, for example, about 150 Å to about 500 Å. If the thickness of the ETL is within the ranges described above, excellent electron transporting characteristics may be obtained without a substantial increase in driving voltage.

Also, the ETL may include an electron transporting organic compound and a metal-containing material. The metal-containing material may include a Li complex. Non-limiting examples of the Li complex are lithium quinolate (LiQ), Compound 103 below, and the like:

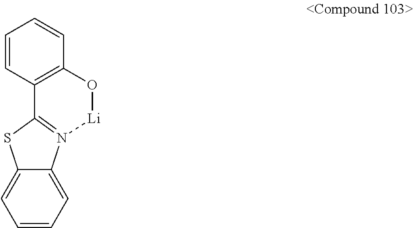

An electron injection layer (EIL) may be deposited on the ETL by using a material that allows electrons to be easily injected from an anode. A material for forming the EIL is not particularly limited.

As the EIL forming material group, any known electron injection layer material group, such as LiF, NaCl group, CsF, Li₂O, or BaO, may be used. If the EIL may be formed by vacuum deposition, the deposition conditions may be similar to those applied to form the HIL GROUP, although the deposition or coating conditions may vary according to the material that is used to form the EIL.

A thickness of the EIL may be from about 1 Å to about 100 Å, for example, about 3 Å to about 90 Å. If the thickness of the EIL is within the ranges described above, excellent electron injection characteristics may be obtained without a substantial increase in driving voltage.

The second electrode 17 is formed on the organic layer 15. The second electrode 17 may be a cathode as an electron injection electrode, and in this case, a low work function metal group, alloy, electrically conductive compound, and a mixture thereof may be used as a second electrode material. In detail group, lithium (Li), magnesium (Mg), aluminum (Al), aluminum-lithium (Al—Li), calcium (Ca), magnesium-indium (Mg—In), magnesium-silver (Mg—Ag), etc. may be formed as a thin film for use as a reflection electrode. Also, if the organic light-emitting diode is used in a top-emission light-emitting device, the second electrode 17 may be formed as a transmission electrode by using ITO or IZO.

Hereinafter, an organic light-emitting diode according to an embodiment of the present invention will be described in detail with reference to Synthesis Examples and Examples. However, the present invention is not limited to Synthesis Example, and Example below.

### SYNTHESIS EXAMPLE

### Synthesis Example 1

### Synthesis of Compound 10

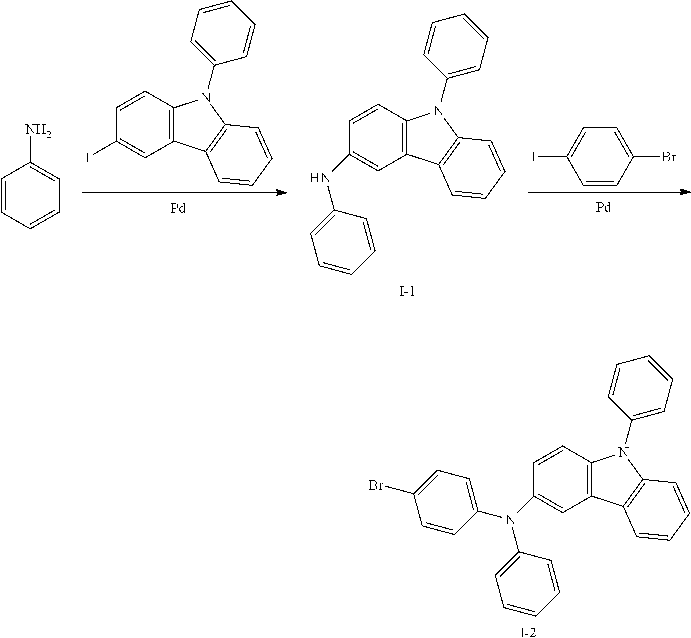

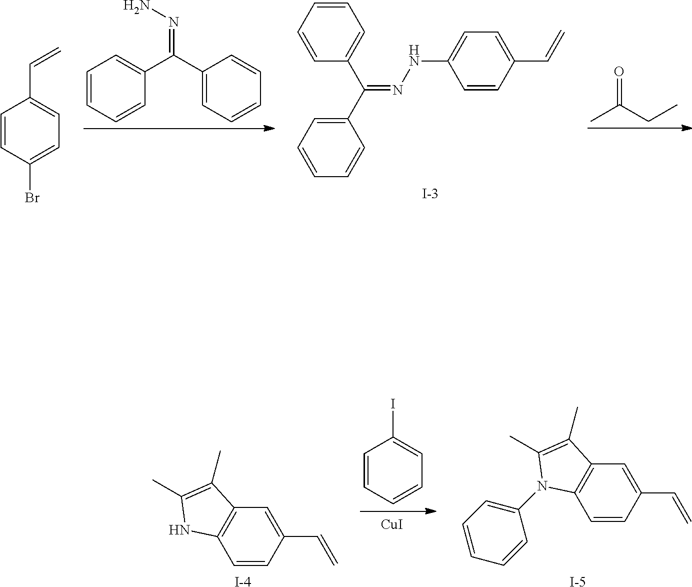

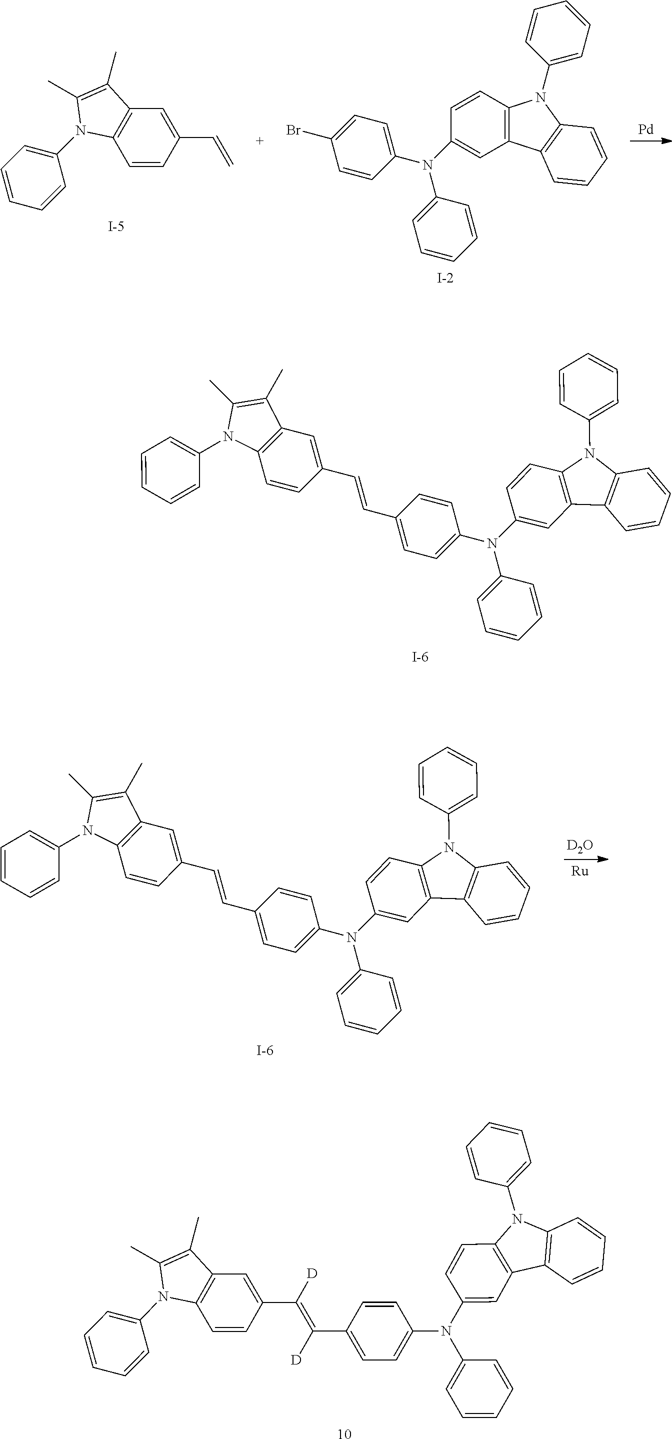

Synthesis of Intermediate I-1

0.93 g (15 mmol) of aniline, 3.69 g (10 mmol) of 3-iodo-9-phenyl-carbazole, 0.83 g (0.2 mmol) of Pd₂(dba)₃, 0.040 g (0.2 mmol) of PtBu₃, and 13.9 g (20 mmol) of KOtBu were dissolved in 60 ml of toluene, and then the mixture was stirred at a temperature of 85° C. for 4 hours. The reaction solution was cooled to room temperature, and then extracted three times with 100 ml of water and 100 ml of diethylether. A collected organic layer was dried with a magnesium sulfate and the residual obtained by evaporating a solvent was separation-purified by silicagel column chromatography to obtain 2.63 g (Yield 79%) of Intermediate I-1. The formed compound was confirmed by MS/FAB. C₂₄H₁₈N₂: calc. 334.14, found 334.21.

Intermediate I-2

Intermediate I-2 was prepared in the same manner as used to prepare Intermediate 1-1, except that Intermediate I-1 was used instead of aniline and 1-bromo-4-iodobenzene was used instead of 3-iodo-9-phenyl-carbazole. The formed compound was confirmed by MS/FAB. C₃₀H₂₁BrN₂: calc. 488.08, found 488.31.

Synthesis of Intermediate I-3

5.49 g (30 mmol) of 4-bromostyrene, 7.1 g (36 mmol) of benzophenone hydrazone, 4.3 g (45 mmol) of t-BuONa, 0.13 g (0.6 mmol) of Pd(OAc)₂, and 0.29 g (0.6 mmol) of 2-dicyclohexylphosphino-2′,4′,6′-triisopropylbiphenyl were dissolved in 60 ml of toluene group, and then the mixture was stirred at a temperature of 90° C. for 3 hours. The reaction product was cooled to room temperature and then distilled water was added thereto, followed by extraction two times with 100 ml of diethylether and once with 100 ml of dichloromethane. A collected organic layer was dried with a magnesium sulfate and the residual obtained by evaporating a solvent was separation-purified by silicagel column chromatography to obtain 7.61 g (Yield 85%) of Intermediate I-3. The formed compound was confirmed by MS/FAB. C₂₁H₁₈N₂: calc. 488.08, found 488.31

Synthesis of Intermediate I-4

80 ml of methylethylketone was added to a mixture including 5.96 g (20 mmol) of Intermediate I-3 and 7.60 g (40 mmol) of p-toluenesulfonic acid dehydrate and then stirred at a temperature of 110° C. for 24 hours. The reaction product was cooled to room temperature and then distilled water was added thereto, followed by extraction two times with 100 ml of diethylether and two times with 100 ml of dichloromethane. A collected organic layer was dried with a magnesium sulfate and the residual obtained by evaporating a solvent was separation-purified by silicagel column chromatography to obtain 2.39 g (Yield 70%) of Intermediate I-4. The formed compound was confirmed by MS/FAB. C₁₂H₁₃N₂: calc. 171.10, found 171.32

Synthesis of Intermediate I-5

1.71 g (10.0 mmol) of Intermediate I-4, 3.06 g (15.0 mmol) of iodobenzene, 0.19 g (1.0 mmol) of CuI, 0.05 g (0.2 mmol) of 18-crown-6 and 4.15 g (30.0 mmol) of K₂CO₃ were dissolved in 30 ml of 1,3-dimethyl-3,4,5,6-tetrahydro-2(1H)-pyrimidinone (DMPU) and then stirred at a temperature of 170° C. for 12 hours. The reaction product was cooled to room temperature and extracted three times with 50 ml of water and 50 ml of chloromethane. A collected organic layer was dried with a magnesium sulfate and the residual obtained by evaporating a solvent was separation-purified by silicagel column chromatography to obtain 2.27 g (Yield 92%) of Intermediate I-5. The formed compound was confirmed by MS/FAB. C₁₈H₁₇N: calc. 247.13, found 247.40

Synthesis of Intermediate I-6

1.24 g (5 mmol) of Intermediate I-5, 2.45 g (5 mmol) of Intermediate I-2, 0.056 g (0.25 mmol) of Pd(OAc)₂, 0.76 g (0.25 mmol) of (p-tolyl)₃P (Tri(o-tolyl)phosphine), and 1.019 g (10 mmol) of triethylamine were dissolved in 100 ml of dimethylacetamide (DMAc) and stirred at a temperature of 85° C. for 4 hours. The reaction product was cooled to room temperature and extracted three times with 100 ml of water and 100 ml of diethylether. A collected organic layer was dried with a magnesium sulfate and the residual obtained by evaporating a solvent was separation-purified by silicagel column chromatography to obtain 1.51 g (Yield 46%) of Intermediate I-6. The formed compound was confirmed by MS/FAB. C₄₈H₃₇N₃: calc. 655.29, found 655.38

Synthesis of Compound 10

1.84 g (2.8 mmol) of Intermediate I-6, 0.081 g (0.08 mmol) of (carbonyl)chloro(hydrido)tris(triphenylphosphine)ruthenium(II), 0.56 g (28 mmol) of D₂O were dissolved in 30 ml of 1,4-dioxane and then stirred at a temperature of 80° C. for 12 hours. The reaction product was cooled to room temperature and a solvent was removed, followed by extraction three times with 50 ml of water and 50 ml of dichloromethane. A collected organic layer was dried with a magnesium sulfate and the residual obtained by evaporating a solvent was separation-purified by silicagel column chromatography to obtain 1.30 g (Yield 71%) of Compound 10. The formed compound was confirmed by MS/FAB.

C₄₈H₃₆D₂N₃: calc. 657.31, found 657.42 ¹H NMR (CDCl₃, 400 MHz) δ(ppm) 8.23-8.19 (m, 1H), 7.60-7.59 (m, 1H), 7.51-7.46 (m, 6H), 7.43-7.23 (m, 11H), 7.15-7.13 (m, 1H), 7.09-7.04 (m, 2H), 6.98 (d, 1H), 6.87-6.81 (m, 3H), 6.66-6.63 (m, 1H), 6.31-6.29 (m, 2H), 2.33-2.31 (m, 6H).

### Synthesis Example 2

### Synthesis of Compound 28

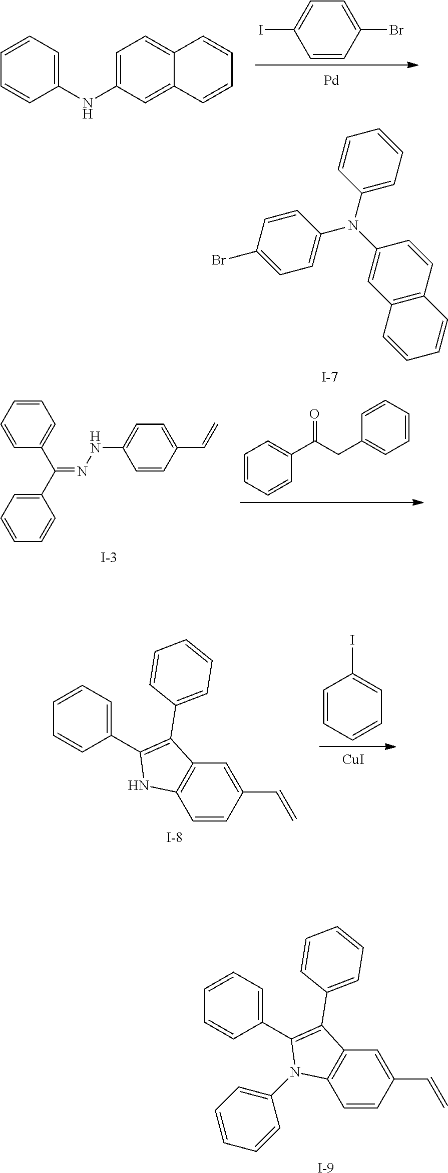

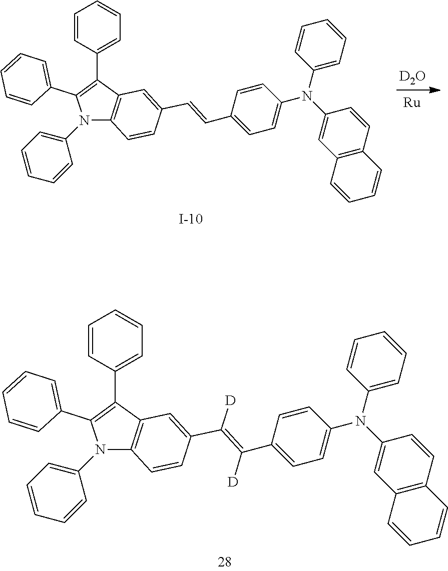

Synthesis of Intermediate I-7

Intermediate I-7 was prepared in the same manner as used to prepare Intermediate I-2 of Synthesis Example 1, except that N-phenyl-2-naphthylamine was used instead of Intermediate I-1. The formed compound was confirmed by MS/FAB. C₂₂H₁₆BrN₂: calc. 373.04, found 373.19

Synthesis of Intermediate I-8

80 ml of ethanol and 80 ml of toluene were added to a mixture including 5.96 g (20 mmol) of Intermediate I-3 of Synthesis Example 1, 7.6 g (40 mmol) of p-toluenesulfonic acid dehydrate, and 15.70 g (80 mmol) of benzylphenylketone, and then stirred at a temperature of 110° C. for 24 hours. The reaction product was cooled to room temperature and distilled water was added thereto, followed by extraction two times with 100 ml of diethylether and two times with 100 ml of dichloromethane. A collected organic layer was dried with a magnesium sulfate and the residual obtained by evaporating a solvent was separation-purified by silicagel column chromatography to obtain 4.13 g (Yield 70%) of Intermediate I-8. The formed compound was confirmed by MS/FAB. C₂₂H₁₇N: calc. 295.13, found 295.21.

Synthesis of Intermediate I-9

Intermediate I-9 was prepared in the same manner as used to prepare Intermediate 1-5 of Synthesis Example 1, except that Intermediate I-8 was used instead of Intermediate I-4. The formed compound was confirmed by MS/FAB. C₂₈H₂₁N: calc. 371.16, found 371.25.

Synthesis of Intermediate I-10

Intermediate I-10 was prepared in the same manner as used to prepare Intermediate 1-6 of Synthesis Example 1, except that Intermediate I-9 was used instead of Intermediate I-5, and Intermediate I-7 was used instead of Intermediate I-2. The formed compound was confirmed by MS/FAB. C₅₀H₃₆N₂: calc. 664.28, found 664.36

Synthesis of Compound 28

Compound 28 was prepared in the same manner as used to prepare Compound 10 of Synthesis Example 1, except that Intermediate I-10 was used instead of Intermediate I-6. The formed compound was confirmed by MS/FAB.

C₅₀H₃₄D₂N₂: calc. 666.30, found 666.40 ¹H NMR (CDCl₃, 400 MHz) δ(ppm) 7.79-7.75 (m, 2H), 7.65 (d, 1H), 7.62-7.27 (m, 16H), 7.13-7.02 (m, 5H), 6.79-6.76 (m, 2H), 6.66-6.63 (m, 1H), 6.25-6.22 (m, 2H).

### Synthesis Example 3

### Synthesis of Compound 35

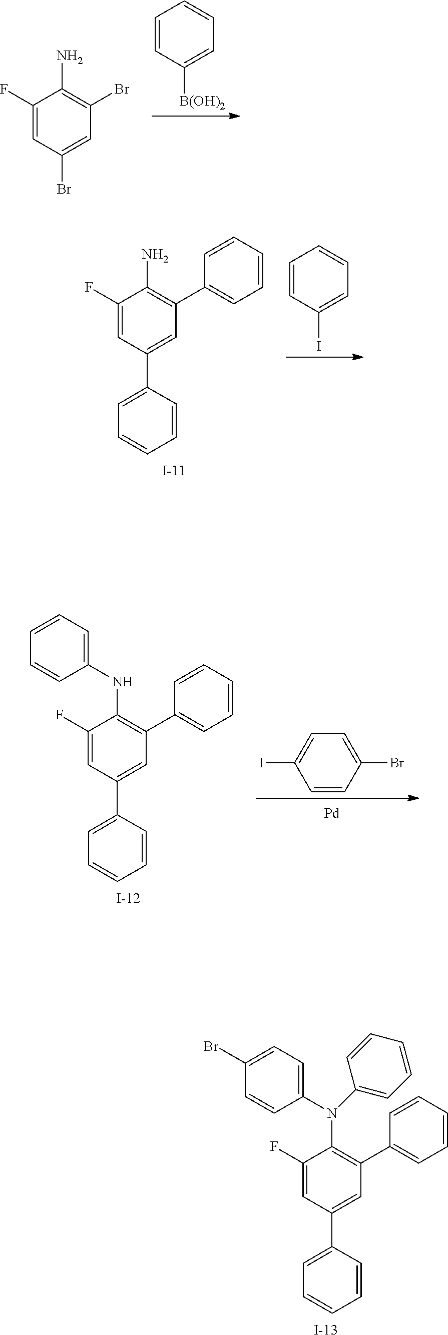

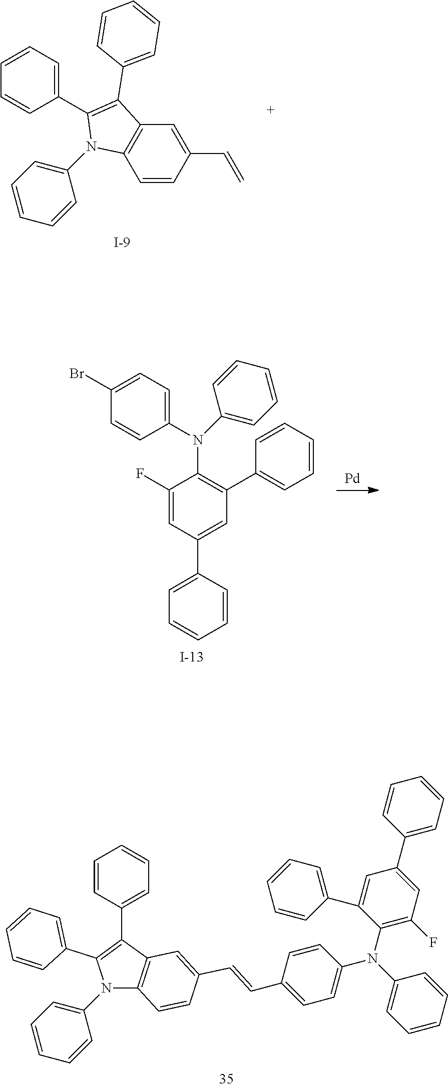

Synthesis of Intermediate I-11

5.37 g (20.0 mmol) of 2,4-dibromo-6-fluoro-phenylamine, 4.88 g (40.0 mmol) of a phenylboronic acid, 1.15 g (1.0 mmol) of tetrakis(triphenylphosphine)palladium (Pd(PPh₃)₄) and 8.29 g (60.0 mmol) of K₂CO₃ were dissolved in 60 ml of a mixed solution of THF/H₂O (volumetric ratio of 2/1), and then stirred at a temperature of 70° C. for 5 hours. The reaction solution was cooled to room temperature and 40 ml of water was added, followed by extraction three times with 50 ml of ethylether. A collected organic layer was dried with a magnesium sulfate and the residual obtained by evaporating a solvent was separation-purified by silicagel column chromatography to obtain 3.95 g (Yield 75%) of Intermediate I-11. The formed compound was confirmed by MS/FAB. C₁₈H₁₄FN: calc. 263.11, found 263.25.

Synthesis of Intermediate I-12

Intermediate I-12 was prepared in the same manner as used to prepare Intermediate 1-1 of Synthesis Example 1, except that Intermediate I-11 was used instead of aniline and 3 iodobenzene was used instead of-iodo-9-phenyl-carbazole. The formed compound was confirmed by MS/FAB. C₂₄H₁₈FN: calc. 339.14, found 339.29.

Synthesis of Intermediate I-13

Intermediate I-13 was prepared in the same manner as used to prepare Intermediate 1-2 of Synthesis Example 1 except that Intermediate I-12 was used instead of Intermediate I-1, and 1-bromo-4-iodobenzene was used. The formed compound was confirmed by MS/FAB. C₃₀H₁₁BrFN: calc. 493.08, found 493.15.

Synthesis of Compound 35

Compound 35 was prepared in the same manner as used to prepare Intermediate 1-6 of Synthesis Example 1, except that Intermediate I-9 was used instead of Intermediate I-5, and Intermediate I-13 was used instead of Intermediate I-2. The formed compound was confirmed by MS/FAB.

C₅₈H₄₁FN₂: calc. 784.32, found 784.41 ¹H NMR (CDCl₃, 400 MHz) δ(ppm) 7.76-7.75 (m, 1H), 7.72-7.69 (m, 2H), 7.65-7.29 (m, 27H), 7.23-7.19 (m, 1H), 7.13-7.10 (m, 1H), 7.08-7.02 (m, 4H), 6.68-6.60 (m, 3H), 6.15-6.11 (m, 2H).

### Synthesis Example 4

### Synthesis of Compound 53

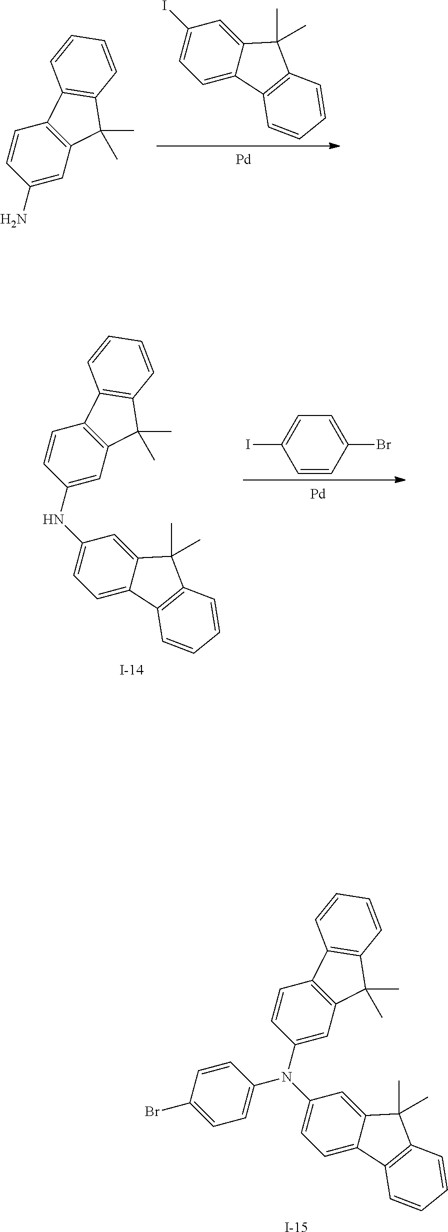

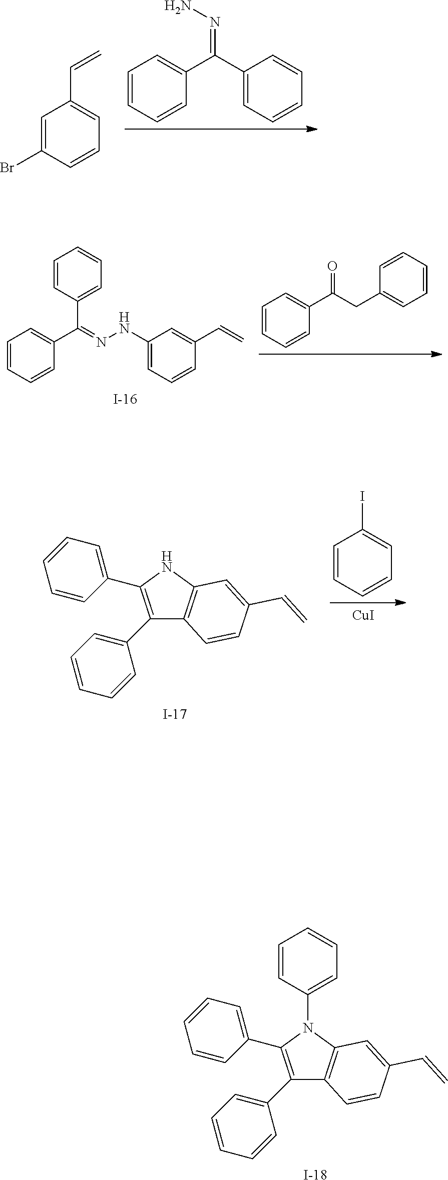

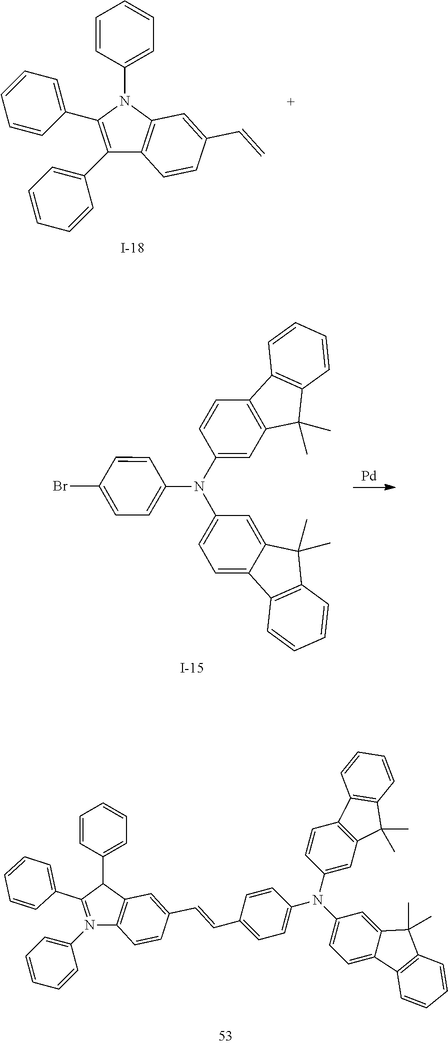

Synthesis of Intermediate I-14

Intermediate I-14 was prepared in the same manner as used to prepare Intermediate 1-1 of Synthesis Example 1, except that 2-amino-9,9-dimethylfluorene was used instead of aniline and 2-iodo-9,9-dimethylfluorene was used instead of 3-iodo-9-phenyl-carbazole. The formed compound was confirmed by MS/FAB. C₃₀H₂₇N: calc. 401.21, found 401.32.

Synthesis of Intermediate I-15

Intermediate I-15 was prepared in the same manner as used to prepare Intermediate 1-2 of Synthesis Example 1, except that Intermediate I-14 was used instead of Intermediate I-1. The formed compound was confirmed by MS/FAB. C₃₆H₃₀BrN: calc. 555.12, found 555.24

Synthesis of Intermediate I-16

Intermediate I-16 was prepared in the same manner as used to prepare Intermediate 1-3 of Synthesis Example 1, except that 3-bromostyrene was used instead of 4-bromostyrene. The formed compound was confirmed by MS/FAB. C₂₁H₁₈N₂: calc. 298.14, found 298.25

Synthesis of Intermediate I-17

Intermediate I-17 was prepared in the same manner as used to prepare Intermediate 1-8 of Synthesis Example 2, except that Intermediate I-16 was used instead of Intermediate I-3. The formed compound was confirmed by MS/FAB. C₂₂H₁₇N: calc. 295.16, found 295.29.

Synthesis of Intermediate I-18

Intermediate I-18 was prepared in the same manner as used to prepare Intermediate 1-5 of Synthesis Example 1, except that Intermediate I-17 was used instead of Intermediate I-4. The formed compound was confirmed by MS/FAB. C₂₈H₂₁N: calc. 371.16, found 371.25.

Synthesis of Compound 53

Compound 53 was prepared in the same manner as used to prepare Intermediate 1-6 of Synthesis Example 1, except that Intermediate I-18 was used instead of Intermediate I-5, and Intermediate I-15 was used instead of Intermediate I-2. The formed compound was confirmed by MS/FAB.

C₆₄H₅₀N₂: calc. 846.39, found 846.45 ¹H NMR (CDCl₃, 400 MHz) δ(ppm) 7.85-7.84 (m, 1H), 7.83-7.81 (m, 2H), 7.78-7.77 (m, 1H), 7.76-7.75 (m, 1H), 7.60-7.56 (m, 4H), 7.51-7.48 (m, 2H), 7.45-7.42 (m, 2H), 7.38-7.29 (m, 10H), 7.26-7.21 (m, 3H), 7.16-7.08 (m, 4H), 6.95-6.89 (m, 2H), 6.75-6.71 (m, 4H), 6.45-6.44 (m, 2H), 1.61 (s, 12H).

### Synthesis Example 5

### Synthesis of Compound 67

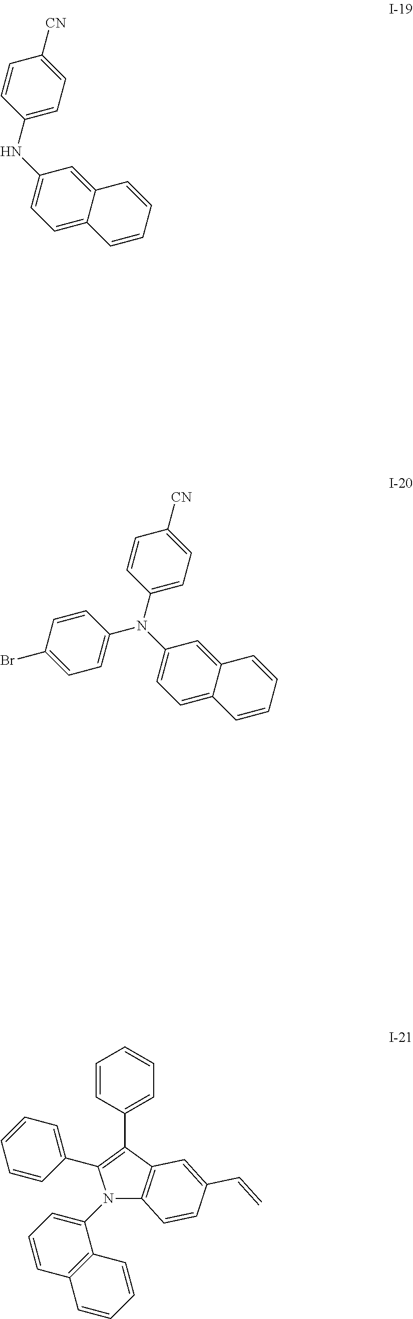 [SMILES: N#Cc1ccc(Nc2ccc3ccccc3c2)cc1 ; N#Cc1ccc(N(c2ccc(Br)cc2)c2ccc3ccccc3c2)cc1 ; C=Cc1ccc2c(c1)c(-c1ccccc1)c(-c1ccccc1)n2-c1cccc2ccccc12]

Synthesis of Intermediate I-19

Intermediate I-19 was prepared in the same manner as used to prepare Intermediate 1-1 of Synthesis Example 1, except that 2-aminonaphthalene was used instead of aniline, and 4-bromobenzonitrile was used instead of 3-iodo-9-phenyl-carbazole. The formed compound was confirmed by MS/FAB. C₁₇H₁₂N₂: calc. 244.10, found 244.20.

Synthesis of Intermediate I-20

Intermediate I-20 was prepared in the same manner as used to prepare Intermediate 1-2 of Synthesis Example 1, except that Intermediate I-19 was used instead of Intermediate I-1. The formed compound was confirmed by MS/FAB. C₂₃H₁₅BrN₂: calc. 398.04, found 398.12.

Synthesis of Intermediate I-21

Intermediate I-21 was prepared in the same manner as used to prepare Intermediate 1-5 of Synthesis Example 1, except that Intermediate I-17 was used instead of Intermediate I-4 and 1-iodonaphthalene was used instead of iodobenzene. The formed compound was confirmed by MS/FAB. C₃₂H₂₃N: calc. 421.18, found 421.26.

Synthesis of Compound 67

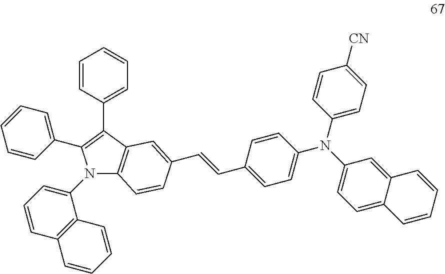 [SMILES: N#Cc1ccc(N(c2ccc(/C=C/c3ccc4c(c3)c(-c3ccccc3)c(-c3ccccc3)n4-c3cccc4ccccc34)cc2)c2ccc3ccccc3c2)cc1]

Compound 67 was prepared in the same manner as used to prepare Intermediate 1-6 of Synthesis Example 1, except that Intermediate I-21 was used instead of Intermediate I-5, and Intermediate I-20 was used instead of Intermediate I-2. The formed compound was confirmed by MS/FAB.

C₅₅H₃₇N₃: calc. 739.29, found 739.38 ¹H NMR (CDCl₃, 400 MHz) δ(ppm) 7.93-7.91 (m, 1H), 7.88-7.86 (m, 1H), 7.78-7.76 (m, 1H), 7.73-7.71 (m, 1H), 7.69-7.64 (m, 2H), 7.59-7.52 (m, 6H), 7.50-7.32 (m, 16H), 7.28-7.24 (m, 2H), 7.13-7.11 (m, 2H), 6.92-6.90 (m, 1H), 6.85-6.78 (m, 4H).

### Synthesis Example 6

### Synthesis of Compound 77

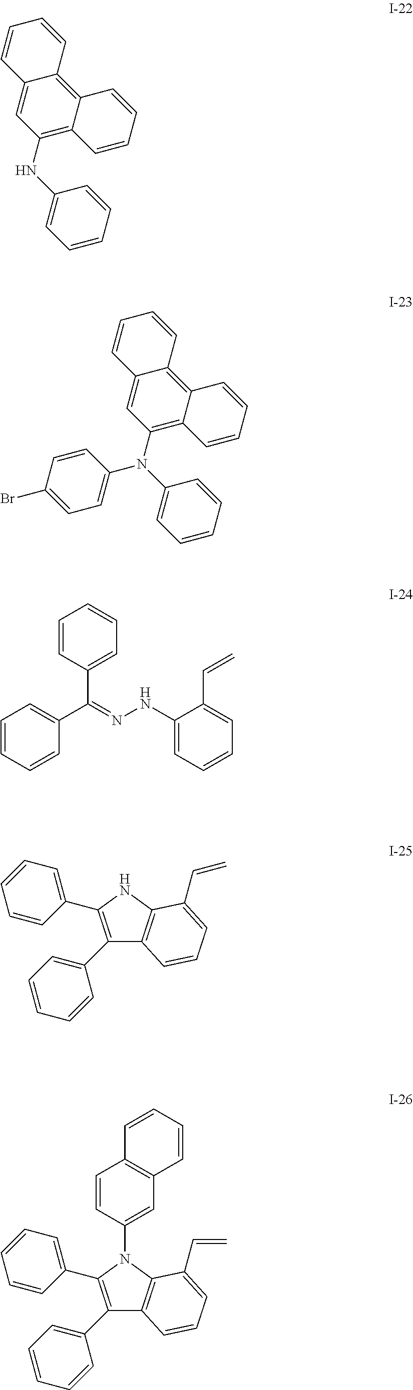 [SMILES: c1ccc(Nc2cc3ccccc3c3ccccc23)cc1 ; Brc1ccc(N(c2ccccc2)c2cc3ccccc3c3ccccc23)cc1 ; C=Cc1ccccc1NN=C(c1ccccc1)c1ccccc1 ; C=Cc1cccc2c(-c3ccccc3)c(-c3ccccc3)[nH]c12 ; C=Cc1cccc2c(-c3ccccc3)c(-c3ccccc3)n(-c3ccc4ccccc4c3)c12]

Synthesis of Intermediate I-22

Intermediate I-22 was prepared in the same manner as used to prepare Intermediate 1-1 of Synthesis Example 1, except that 3-bromophenanthrene was used instead of 3-iodo-9-phenyl-carbazole. The formed compound was confirmed by MS/FAB. C₂₀H₁₅N: calc. 269.12, found 269.19.

Synthesis of Intermediate I-23

Intermediate I-23 was prepared in the same manner as used to prepare Intermediate 1-2 of Synthesis Example 1, except that Intermediate I-22 was used instead of Intermediate I-1. The formed compound was confirmed by MS/FAB. C₂₆H₁₈BrN: calc. 423.06, found 423.15.

Synthesis of Intermediate I-24

Intermediate I-24 was prepared in the same manner as used to prepare Intermediate 1-3 of Synthesis Example 1, except that 2-bromostyrene was used instead of 4-bromostyrene. The formed compound was confirmed by MS/FAB. C₂₁H₁₈N₂: calc. 298.14, found 298.25.

Synthesis of Intermediate I-25

Intermediate I-25 was prepared in the same manner as used to prepare Intermediate 1-8 of Synthesis Example 2, except that Intermediate I-24 was used instead of Intermediate I-3. The formed compound was confirmed by MS/FAB. C₂₂H₁₇N: calc. 295.16, found 295.29.

Synthesis of Intermediate I-26

Intermediate I-26 was prepared in the same manner as used to prepare Intermediate 1-5 of Synthesis Example 1, except that Intermediate I-25 was used instead of Intermediate I-4 and 2-iodonaphthalene was used instead of iodobenzene. The formed compound was confirmed by MS/FAB. C₃₂H₂₃N: calc. 421.18, found 421.26.

Synthesis of Compound 77

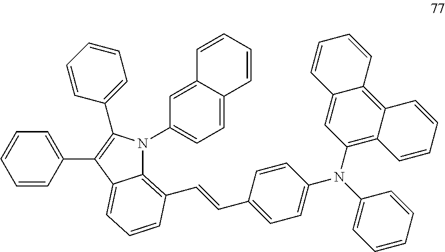 [SMILES: C(=C\c1cccc2c(-c3ccccc3)c(-c3ccccc3)n(-c3ccc4ccccc4c3)c12)/c1ccc(N(c2ccccc2)c2cc3ccccc3c3ccccc23)cc1]

Compound 77 was prepared in the same manner as used to prepare Intermediate 1-6, except that Intermediate I-26 was used instead of Intermediate I-5, and Intermediate I-23 was used instead of Intermediate I-2. The formed compound was confirmed by MS/FAB.

C₅₈H₄₀N₂: calc. 764.31, found 764.40 ¹H NMR (CDCl₃, 400 MHz) ••8.59-8.54 (m, 1H), 8.20-8.17 (m, 1H), 7.96-7.91 (m, 2H), 7.85-7.83 (m, 1H), 7.71-7.29 (m, 24H), 7.07-7.00 (m, 6H), 6.71-6.61 (m, 3H), 6.12-6.09 (m, 2H).

Compounds 1 to 83 were synthesized in the same manner as described above.

### Synthesis Example 7

### Synthesis of Compound 1

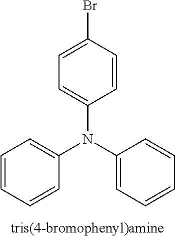 [SMILES: Brc1ccc(N(c2ccccc2)c2ccccc2)cc1]

Compound 1 was prepared in the same manner as used to prepare Intermediate 1-6 of Synthesis Example 1, except that tris(4-bromophenyl)amine was used instead of Intermediate I-2. The formed compound was confirmed by MS/FAB.

C₃₆H₃₀N₂: calc. 490.24, found 490.33 ¹H NMR (CDCl₃, 400 MHz) δ(ppm) 7.60-7.59 (m, 1H), 7.48-7.44 (m, 2H), 7.43-7.39 (m, 2H), 7.37-7.32 (m, 3H), 7.30-7.26 (m, 1H), 7.18-7.15 (m, 2H), 7.08-7.03 (m, 4H), 6.99-6.67 (m, 1H), 6.70-6.62 (m, 4H), 6.16-6.13 (m, 4H), 2.33-2.32 (m, 6H).

### Synthesis Example 8

### Synthesis of Compound 5

Compound 5 was prepared in the same manner as used to prepare Intermediate 1-6 of Synthesis Example 1, except that Intermediate I-20 was used instead of Intermediate I-2. The formed compound was confirmed by MS/FAB.

C₄₁H₃₁N₃: calc. 585.25, found 585.33 ¹H NMR (CDCl₃, 400 MHz) δ(ppm) 7.78-7.75 (m, 1H), 7.65 (d, 1H), 7.60-7.58 (m, 1H), 7.56-7.53 (m, 2H), 7.50-7.46 (m, 2H), 7.43-7.33 (m, 9H), 7.30-7.26 (m, 1H), 7.18-7.15 (m, 2H), 6.98 (d, 1H), 6.91 (dd, 1H), 6.85-6.78 (m, 4H), 2.34-2.32 (m, 6H).

### Synthesis Example 9

### Synthesis of Compound 8

Compound 8 was prepared in the same manner as used to prepare Compound 10 of Synthesis Example 1, except that Intermediate I-15 was used instead of Intermediate I-2. The formed compound was confirmed by MS/FAB.

C₅₄H₄₄D₂N₂: calc. 724.37, found 724.48 ¹H NMR (CDCl₃, 400 MHz) δ(ppm) 7.78-7.76 (m, 2H), 7.60-7.56 (m, 3H), 7.50-7.47 (m, 2H), 7.43-7.40 (m, 2H), 7.38-7.30 (m, 5H), 7.15-7.10 (m, 5H), 6.98 (d, 1H), 6.75-6.72 (m, 4H), 6.45-6.44 (m, 2H), 2.33-2.31 (m, 6H), 1.61 (s, 12H).

### Synthesis Example 10

### Synthesis of Compound 11

Compound 11 was prepared in the same manner as used to prepare Intermediate 1-6 of Synthesis Example 1, except that Intermediate I-13 was used instead of Intermediate I-2. The formed compound was confirmed by MS/FAB.

C₄₈H₃₇FN₂: calc. 660.29, found 660.36 ¹H NMR (CDCl₃, 400 MHz) δ(ppm) 7.72-7.69 (m, 2H), 7.66-7.59 (m, 4H), 7.56-7.49 (m, 5H), 7.44-7.39 (m, 5H), 7.38-7.34 (m, 2H), 7.30-7.26 (m, 2H), 7.16-7.03 (m, 5H), 6.98 (d, 1H), 6.68-6.60 (m, 3H), 6.15-6.11 (m, 2H), 2.33-2.31 (m, 6H).

### Synthesis Example 11

### Synthesis of Compound 14

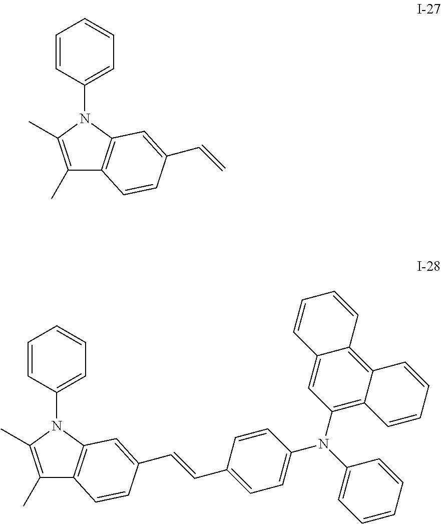 [SMILES: C=Cc1ccc2c(C)c(C)n(-c3ccccc3)c2c1 ; Cc1c(C)n(-c2ccccc2)c2cc(/C=C/c3ccc(N(c4ccccc4)c4cc5ccccc5c5ccccc45)cc3)ccc12]

Intermediate I-27 was prepared in the same manner as used to prepare Intermediate 1-5 of Synthesis Example 1, except that 3-bromostyrene was used instead of 4-bromostyrene. Intermediate I-28 was prepared in the same manner as used to prepare Intermediate I-6 of Synthesis Example 1, except that Intermediate I-27 was used instead of Intermediate 1-5, and Intermediate I-23 of Synthesis Example 6 was used instead of Intermediate 1-2. Then, Compound 14 was prepared in the same manner as used to prepare Compound 10 of Synthesis Example 1, except that Intermediate I-28 was used instead of Intermediate I-6. The formed compound was confirmed by MS/FAB.

C₄₄H₃₂D₂N₂: calc. 592.28, found 592.35 ¹H NMR (CDCl₃, 400 MHz) δ(ppm) 8.60-8.57 (m, 1H), 8.20-8.17 (m, 1H), 7.96-7.93 (m, 1H), 7.72-7.67 (m, 2H), 7.60-7.54 (m, 2H), 7.46-7.32 (m, 8H), 7.30-7.27 (m, 1H), 7.14-7.11 (m, 1H), 7.08-7.03 (m, 3H), 6.98-6.97 (m, 1H), 6.66-6.61 (m, 3H), 6.12-6.09 (m, 2H), 2.54 (s, 3H), 2.33 (s, 3H).

### Synthesis Example 12

### Synthesis of Compound 16

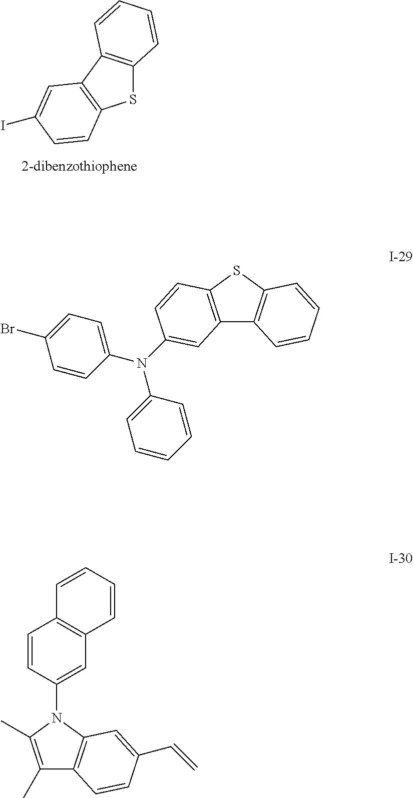 [SMILES: Ic1ccc2sc3ccccc3c2c1 ; Brc1ccc(N(c2ccccc2)c2ccc3sc4ccccc4c3c2)cc1 ; C=Cc1ccc2c(C)c(C)n(-c3ccc4ccccc4c3)c2c1]

Intermediate I-29 was prepared in the same manner as used to prepare Intermediate 1-2 of Synthesis Example 1, except that 2-dibenzothiophene was used instead of 3-iodo-9-phenylcarbazole. Intermediate I-30 was prepared in the same manner as used to prepare Intermediate 1-5 of Synthesis Example 1, except that 3-bromostyrene was used instead of 4-bromostyrene, and 2-iodonaphthalene was used instead of Iodobenzene. Then, Compound 16 was prepared in the same manner as used to prepare Intermediate I-6 of Synthesis Example 1, except that Intermediate I-29 was used instead of Intermediate I-2, and Intermediate I-30 was used instead of Intermediate I-5. The formed compound was confirmed by MS/FAB.

C₄₆H₃₄N₂S: calc. 646.24, found 646.33 ¹H NMR (CDCl₃, 400 MHz) δ(ppm) 8.06-8.04 (m, 1H), 7.87-7.81 (m, 3H), 7.72-7.70 (m, 1H), 7.67-7.65 (m, 1H), 7.62-7.58 (m, 2H), 7.55-7.36 (m, 9H), 7.23-7.21 (m, 1H), 7.09-7.00 (m, 4H), 6.87-6.81 (m, 3H), 6.66-6.63 (m, 1H), 6.32-6.29 (m, 2H), 2.54 (s, 3H), 2.33 (s, 3H).

### Synthesis Example 13

### Synthesis of Compound 17

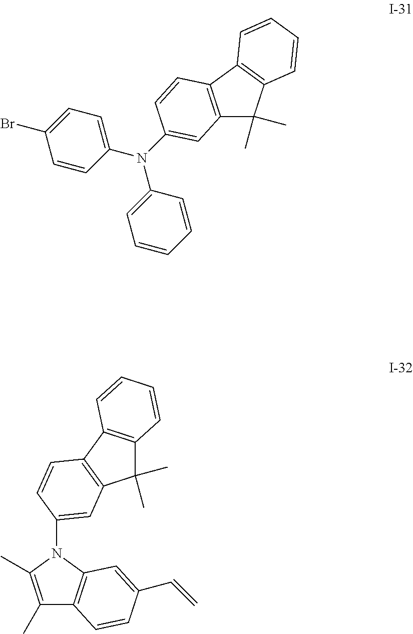 [SMILES: CC1(C)c2ccccc2-c2ccc(N(c3ccccc3)c3ccc(Br)cc3)cc21 ; C=Cc1ccc2c(C)c(C)n(-c3ccc4c(c3)C(C)(C)c3ccccc3-4)c2c1]

Intermediate I-31 was prepared in the same manner as used to prepare Intermediate 1-2 of Synthesis Example 1, except that 2-iodo-9,9-dimethylfluorene was used instead of 3-iodo-9-phenylcarbazole. Also, Intermediate I-32 was prepared in the same manner as used to prepare Intermediate 1-5 of Synthesis Example 1, except that 3-bromostyrene was used instead of 4-bromostyrene and 2-iodo-9,9-dimethylfluorene was used instead of Iodobenzene. Subsequently, Compound 17 was prepared in the same manner as used to prepare Intermediate I-6 of Synthesis Example 1, except that Intermediate I-31 was used instead of Intermediate I-2, and Intermediate I-32 was used instead of Intermediate I-5. The formed compound was confirmed by MS/FAB.

C₅₄H₄₆N₂: calc. 722.36, found 722.45 ¹H NMR (CDCl₃, 400 MHz) δ(ppm) 7.87-7.85 (m, 1H), 7.78-7.76 (m, 1H), 7.64 (d, 1H), 7.55 (d, 1H), 7.50-7.46 (m, 2H), 7.39-7.30 (m, 3H), 7.23-7.16 (m, 4H), 7.13-6.97 (m, 6H), 6.86-6.85 (m, 1H), 6.73-6.63 (m, 5H), 6.39-6.39 (m, 1H), 6.24-6.20 (m, 2H), 2.54 (s, 3H), 2.31 (s, 3H), 1.64 (s, 6H), 1.61 (s, 6H).

### Synthesis Example 14

### Synthesis of Compound 18

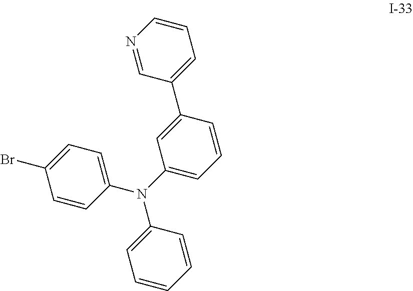 [SMILES: Brc1ccc(N(c2ccccc2)c2cccc(-c3cccnc3)c2)cc1]

Intermediate I-33 was prepared in the same manner as used to prepare Intermediate 1-2 of Synthesis Example 1, except that 3-(3-iodophenyl)pyridine was used instead 3-iodo-9-phenylcarbazole. Then, Compound 18 was prepared in the same manner as used to prepare Intermediate I-6 of Synthesis Example 1, except that Intermediate I-33 was used instead of Intermediate I-2, and Intermediate I-27 of Synthesis Example 11 was used instead of Intermediate I-5. The formed compound was confirmed by MS/FAB.

C₄₁H₃₃N₃: calc. 567.26, found 567.33 ¹H NMR (CDCl₃, 400 MHz) δ(ppm) 8.86-8.84 (m, 1H), 8.66-8.64 (m, 1H), 7.86-7.83 (m, 1H), 7.50-7.46 (m, 2H), 7.44-7.32 (m, 6H), 7.28-7.26 (m, 1H), 7.23-7.15 (m, 4H), 7.09-6.97 (m, 5H), 6.79-6.76 (m, 2H), 6.66-6.63 (m, 1H), 6.25-6.22 (m, 2H), 6.17-6.15 (m, 1H), 2.54 (s, 3H), 2.32 (s, 3H).

### Synthesis Example 15

### Synthesis of Compound 21

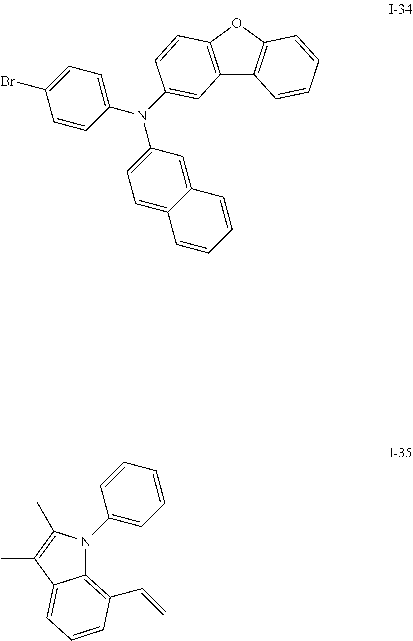 [SMILES: Brc1ccc(N(c2ccc3ccccc3c2)c2ccc3oc4ccccc4c3c2)cc1 ; C=Cc1cccc2c(C)c(C)n(-c3ccccc3)c12]

Intermediate I-34 was prepared in the same manner as used to prepare Intermediate 1-2 of Synthesis Example 1, except that naphthalen-2-amine was used instead of aniline and 1-iodo-dibenzofuran was used instead of 3-iodo-9-phenylcarbazole. Intermediate I-35 was prepared in the same manner as used to prepare Intermediate 1-5 of Synthesis Example 1, except that 2-bromostyrene was used instead of 4-bromostyrene. Then, Compound 21 was prepared in the same manner as used to prepare Intermediate I-6 of Synthesis Example 1, except that Intermediate I-34 was used instead of Intermediate I-2, and Intermediate I-35 was used instead of Intermediate I-5. The formed compound was confirmed by MS/FAB.

C₄₆H₃₄N₂O: calc. 630.26, found 630.32 ¹H NMR (CDCl₃, 400 MHz) δ(ppm) 7.84-7.82 (m, 1H), 7.78-7.76 (m, 1H), 7.74-7.72 (m, 1H), 7.70-7.68 (m, 1H), 7.62-7.61 (m, 1H), 7.58-7.50 (m, 7H), 7.44-7.30 (m, 9H), 7.25-7.23 (m, 1H), 7.14-7.07 (m, 2H), 6.94-6.88 (m, 5H), 2.37 (s, 3H), 2.33 (s, 3H).

### Synthesis Example 16

### Synthesis of Compound 24

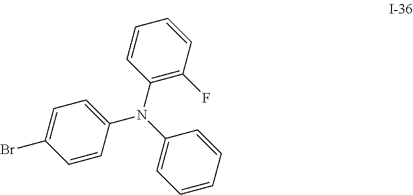 [SMILES: Fc1ccccc1N(c1ccccc1)c1ccc(Br)cc1]

Intermediate I-36 was prepared in the same manner as used to prepare Intermediate 1-2 of Synthesis Example 1, except that 1-fluoro-2-iodobenzene was used instead of 3-iodo-9-phenylcarbazole. Compound 24 was prepared in the same manner as used to prepare Intermediate I-6 of Synthesis Example 1, except that Intermediate I-36 was used instead of Intermediate I-2, and Intermediate I-9 of Synthesis Example 2 was used instead of Intermediate I-5. The formed compound was confirmed by MS/FAB.

C₄₆H₃₃FN₂: calc. 632.26, found 632.32 ¹H NMR (CDCl₃, 400 MHz) δ(ppm) 7.76-7.74 (m, 1H), 7.62-7.57 (m, 2H), 7.49-7.29 (m, 16H), 7.23-7.19 (m, 1H), 7.11-7.02 (m, 5H), 6.98-6.93 (m, 2H), 6.67-6.61 (m, 4H), 6.15-6.12 (m, 2H).

### Synthesis Example 17

### Synthesis of Compound 27

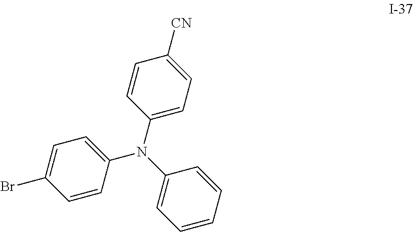 [SMILES: N#Cc1ccc(N(c2ccccc2)c2ccc(Br)cc2)cc1]

Intermediate I-37 was prepared in the same manner as used to prepare Intermediate 1-2 of Synthesis Example 1, except that 4-bromobenzonitrile was used instead of 3-iodo-9-phenylcarbazole. Compound 27 was prepared in the same manner as used to prepare Intermediate I-6 of Synthesis Example 1, except that Intermediate I-37 was used instead of Intermediate I-2, and Intermediate I-9 of Synthesis Example 2 was used instead of Intermediate I-5. The formed compound was confirmed by MS/FAB.

C₄₇H₃₃N₃: calc. 639.26, found 639.34 ¹H NMR (CDCl₃, 400 MHz) δ(ppm) 7.76-7.75 (m, 1H), 7.61-7.57 (m, 3H), 7.50-7.29 (m, 17H), 7.23-7.19 (m, 1H), 7.08-7.02 (m, 4H), 6.78-6.72 (m, 4H), 6.66-6.63 (m, 1H), 6.23-6.19 (m, 2H).

### Synthesis Example 18

### Synthesis of Compound 32

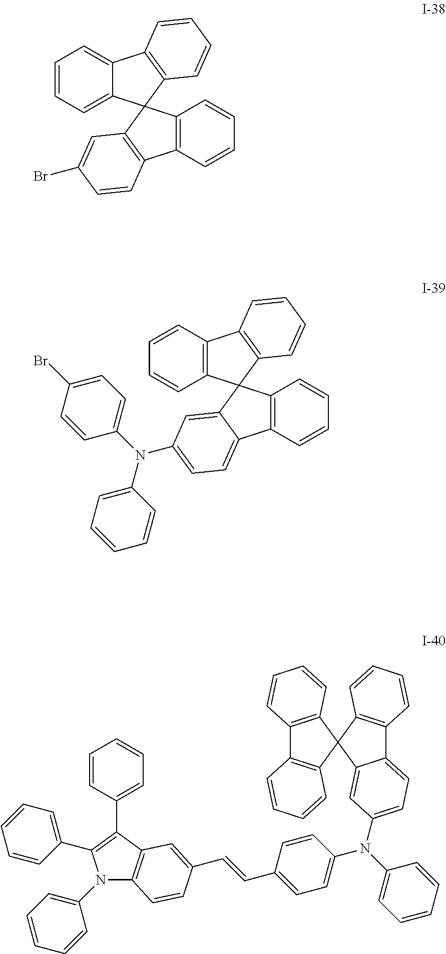 [SMILES: Brc1ccc2c(c1)C1(c3ccccc3-c3ccccc31)c1ccccc1-2 ; Brc1ccc(N(c2ccccc2)c2ccc3c(c2)C2(c4ccccc4-c4ccccc42)c2ccccc2-3)cc1 ; C(=C\c1ccc2c(c1)c(-c1ccccc1)c(-c1ccccc1)n2-c1ccccc1)/c1ccc(N(c2ccccc2)c2ccc3c(c2)C2(c4ccccc4-c4ccccc42)c2ccccc2-3)cc1]

Intermediate I-39 was prepared in the same manner as used to prepare Intermediate 1-2 of Synthesis Example 1, except that Intermediate I-38 was used instead of 3-iodo-9-phenylcarbazole. Intermediate I-40 was prepared in the same manner as used to prepare Intermediate I-6 of Synthesis Example 1, except that Intermediate I-39 was used instead of Intermediate I-2, and Intermediate I-9 of Synthesis Example 2 was used instead of Intermediate I-5. Compound 32 was prepared in the same manner as used to prepare Compound 10 of Synthesis Example 1, except that Intermediate I-40 was used instead of Intermediate I-6. The formed compound was confirmed by MS/FAB.

C₆₅H₄₂D₂N₂: calc. 854.36, found 854.44 ¹H NMR (CDCl₃, 400 MHz) δ(ppm) 7.92-7.87 (m, 4H), 7.75-7.74 (m, 1H), 7.62-7.56 (m, 4H), 7.49-7.29 (m, 19H), 7.19-7.15 (m, 2H), 7.09-7.02 (m, 3H), 6.83-6.80 (m, 1H), 6.77-6.70 (m, 4H), 6.66-6.63 (m, 1H), 6.46-6.45 (m, 1H), 6.24-6.21 (m, 2H).

### Synthesis Example 19

### Synthesis of Compound 36

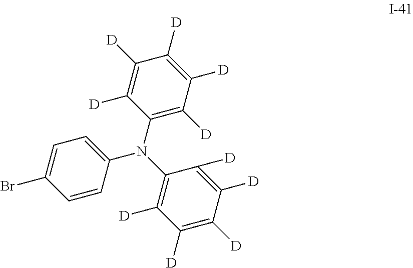 [SMILES: [2H]c1c([2H])c([2H])c(N(c2ccc(Br)cc2)c2c([2H])c([2H])c([2H])c([2H])c2[2H])c([2H])c1[2H]]

Intermediate I-41 was prepared in the same manner as used to prepare Intermediate I-2 of Synthesis Example 1, except that aniline-d5 was used instead of aniline and bromobenzene-d5 was used instead of 3-iodo-9-phenylcarbazole. Compound 36 was prepared in the same manner as used to prepare Compound 10 of Synthesis Example 1, except that Intermediate I-41 was used instead of Intermediate I-2, and Intermediate I-9 of Synthesis Example 2 was used instead of Intermediate I-5. The formed compound was confirmed by MS/FAB.

C₄₆H₂₂D₁₂N₂: calc. 626.34, found 626.44 ¹H NMR (CDCl₃, 400 MHz) δ(ppm) 7.76-7.74 (m, 1H), 7.62-7.54 (m, 2H), 7.48-7.40 (m, 5H), 7.39-7.28 (m, 10H), 7.05-7.02 (m, 2H), 6.69-6.66 (m, 2H).

### Synthesis Example 20

### Synthesis of Compound 38

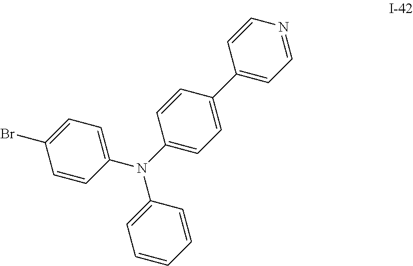 [SMILES: Brc1ccc(N(c2ccccc2)c2ccc(-c3ccncc3)cc2)cc1]

Intermediate I-42 was prepared in the same manner as used to prepare Intermediate 1-2 of Synthesis Example 1, except that 3-(4-iodophenyl)pyridine was used instead of 3-iodo-9-phenylcarbazole. Then, Compound 38 was prepared in the same manner as used to prepare Intermediate I-6 of Synthesis Example 1, except that Intermediate I-42 was used instead of Intermediate I-2, and Intermediate I-9 of Synthesis Example 2 was used instead of Intermediate I-5. The formed compound was confirmed by MS/FAB.

C₅₁H₃₇N₃: calc. 691.29, found 691.37 ¹H NMR (CDCl₃, 400 MHz) δ(ppm) 8.72-8.71 (m, 1H), 7.75-7.74 (m, 1H), 7.62-7.53 (m, 7H), 7.50-7.29 (m, 16H), 7.23-7.19 (m, 1H), 7.08-7.02 (m, 4H), 6.78-6.75 (m, 2H), 6.68-6.63 (m, 3H), 6.22-6.19 (m, 2H).

### Synthesis Example 21

### Synthesis of Compound 40

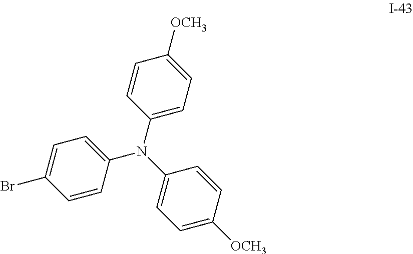 [SMILES: COc1ccc(N(c2ccc(Br)cc2)c2ccc(OC)cc2)cc1]

Intermediate I-43 was prepared in the same manner as used to prepare Intermediate 1-2 of Synthesis Example 1, except that 4-methoxy-phenylamine was used instead of aniline and 1-iodo-4-methoxybenzene was used instead of 3-iodo-9-phenylcarbazole. Then, Compound 40 was prepared in the same manner as used to prepare Intermediate I-6 of Synthesis Example 1, except that Intermediate I-43 was used instead of Intermediate I-2, and Intermediate I-9 of Synthesis Example 2 was used instead of Intermediate I-5. The formed compound was confirmed by MS/FAB.

C₄₈H₃₈N₂O₂: calc. 674.29, found 674.37 ¹H NMR (CDCl₃, 400 MHz) δ(ppm) 7.75-7.74 (m, 1H), 7.64-7.56 (m, 4H), 7.49-7.29 (m, 14H), 7.23-7.19 (m, 1H), 7.14-7.10 (m, 4H), 7.05-7.02 (m, 2H), 6.92-6.88 (m, 4H), 6.82-6.80 (m, 2H), 3.83 (s, 6H).

### Synthesis Example 22

### Synthesis of Compound 46

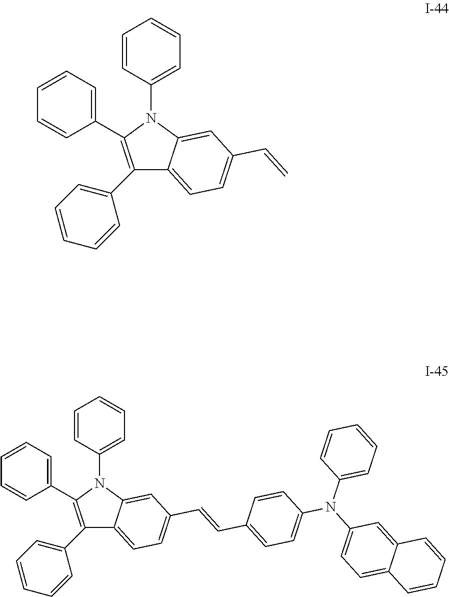 [SMILES: C=Cc1ccc2c(-c3ccccc3)c(-c3ccccc3)n(-c3ccccc3)c2c1 ; C(=C\c1ccc2c(-c3ccccc3)c(-c3ccccc3)n(-c3ccccc3)c2c1)/c1ccc(N(c2ccccc2)c2ccc3ccccc3c2)cc1]

Intermediate I-44 was prepared in the same manner as used to prepare Intermediate 1-5 of Synthesis Example 1, except that 3-bromostyrene was used instead of 4-bromostyrene and benzylphenylketone was used instead of methylethylketone. Intermediate I-45 was prepared in the same manner as used to prepare Intermediate 1-6 of Synthesis Example 1, except that Intermediate I-44 was used instead of Intermediate 1-5, and Intermediate I-7 of Synthesis Example 2 was used instead of Intermediate 1-2. Then, Compound 46 was prepared in the same manner as used to prepare Compound 10 of Synthesis Example 1, except that Intermediate I-45 was used instead of Intermediate I-6. The formed compound was confirmed by MS/FAB.

C₅₄H₃₄D₂N₂: calc. 666.30, found 666.37 ¹H NMR (CDCl₃, 400 MHz) δ(ppm) 7.84-7.81 (m, 1H), 7.78-7.76 (m, 1H), 7.66-7.64 (m, 1H), 7.59-7.52 (m, 5H), 7.48-7.29 (m, 15H), 7.13-7.05 (m, 5H), 6.94-6.89 (m, 2H), 6.79-6.76 (m, 2H), 6.66-6.63 (m, 1H), 6.25-6.23 (m, 1H).

### Synthesis Example 23

### Synthesis of Compound 47

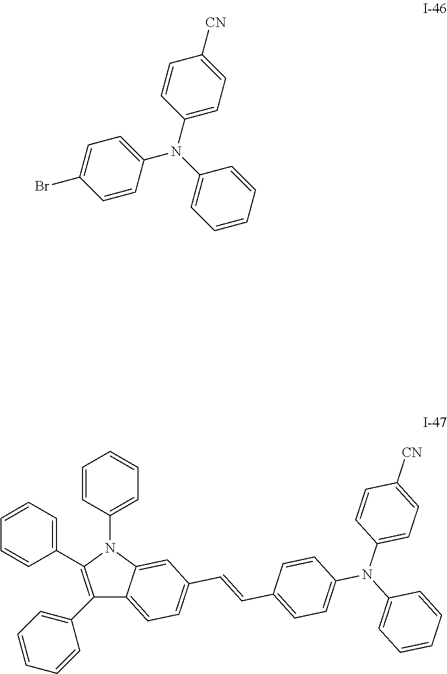 [SMILES: N#Cc1ccc(N(c2ccccc2)c2ccc(Br)cc2)cc1 ; N#Cc1ccc(N(c2ccccc2)c2ccc(/C=C/c3ccc4c(-c5ccccc5)c(-c5ccccc5)n(-c5ccccc5)c4c3)cc2)cc1]

Intermediate I-46 was prepared in the same manner as used to prepare Intermediate 1-2 of Synthesis Example 1, except that 4-bromobenzonitrile was used instead of 3-iodo-9-phenylcarbazole. Intermediate I-47 was prepared in the same manner as used to prepare Intermediate 1-6 of Synthesis Example 1, except that Intermediate I-44 of Synthesis Example 22 was used instead of Intermediate 1-5, and Intermediate I-46 was used instead of Intermediate 1-2. Then, Compound 47 was prepared in the same manner as used to prepare Compound 10 of Synthesis Example 1, except that Intermediate I-47 was used instead of Intermediate I-6. The formed compound was confirmed by MS/FAB.

C₄₇H₃₁D₂N₃: calc. 641.27, found 641.38 ¹H NMR (CDCl₃, 400 MHz) δ(ppm) 7.84-7.81 (m, 2H), 7.61-7.56 (m, 2H), 7.47-7.42 (m, 5H), 7.39-7.29 (m, 9H), 7.11-7.03 (m, 4H), 6.95-6.89 (m, 2H), 6.78-6.73 (m, 4H), 6.66-6.63 (m, 1H), 6.23-6.20 (m, 2H).

### Synthesis Example 24

### Synthesis of Compound 49

Compound 49 was used in the same manner as used to prepare Intermediate 1-6 of Synthesis Example 1, except that Intermediate I-31 of Synthesis Example 13 was used instead of Intermediate 1-2, and Intermediate I-44 of Synthesis Example 22 was used instead of Intermediate 1-5. The formed compound was confirmed by MS/FAB.

C₅₅H₄₂N₂: calc. 730.33, found 730.42 ¹H NMR (CDCl₃, 400 MHz) δ(ppm) 7.84-7.81 (m, 2H), 7.78-7.76 (m, 1H), 7.59-7.54 (m, 3H), 7.49-7.42 (m, 5H), 7.39-7.29 (m, 9H), 7.26-7.22 (m, 3H), 7.13-7.04 (m, 4H), 6.94-6.89 (m, 2H), 6.73-6.63 (m, 4H), 6.39-6.37 (m, 1H), 6.24-6.21 (m, 2H), 1.61 (s, 6H).

### Synthesis Example 25

### Synthesis of Compound 54

Compound 54 was prepared in the same manner as used to prepare Intermediate 1-6 of Synthesis Example 1, except that Intermediate I-13 of Synthesis Example 3 was used instead of Intermediate 1-2, and Intermediate I-44 of Synthesis Example 22 was used instead of Intermediate 1-5. The formed compound was confirmed by MS/FAB.

C₅₈H₃₉D₂FN₂: calc. 846.39, found 846.45 ¹H NMR (CDCl₃, 400 MHz) δ(ppm) 7.85-7.84 (m, 1H), 7.83-7.81 (m, 2H), 7.73-7.69 (m, 2H), 7.66-7.49 (m, 8H), 7.47-7.39 (m, 6H), 7.38-7.29 (m, 7H), 7.13-7.04 (m, 5H), 6.94-6.89 (m, 2H), 6.68-6.60 (m, 4H), 6.15-6.11 (m, 2H).

### Synthesis Example 26

### Synthesis of Compound 57

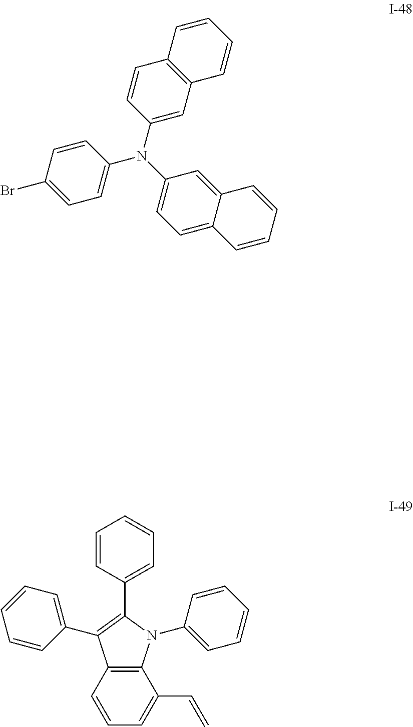 [SMILES: Brc1ccc(N(c2ccc3ccccc3c2)c2ccc3ccccc3c2)cc1 ; C=Cc1cccc2c(-c3ccccc3)c(-c3ccccc3)n(-c3ccccc3)c12]

Intermediate I-48 was prepared in the same manner as used to prepare Intermediate 1-7 of Synthesis Example 2, except that N-(naphthalen-6-yl)naphthalen-2-amine was used instead of N-phenyl-2-naphthylamine. Intermediate I-49 was prepared in the same manner as used to prepare Intermediate 1-5 of Synthesis Example 1, except that 2-bromostyrene was used instead of 4-bromostyrene and benzylphenylketone was used instead of methylethylketone. Then, Compound 57 was prepared in the same manner as used to prepare Compound 53 of Synthesis Example 4, except that Intermediate 1-48 was used instead of Intermediate 1-15, and I-49 was used instead of Intermediate I-18. The formed compound was confirmed by MS/FAB.

C₅₄H₃₈N₂: calc. 714.30, found 714.42 ¹H NMR (CDCl₃, 400 MHz) δ(ppm) 7.78-7.76 (m, 2H), 7.71-7.68 (m, 2H), 7.66-7.65 (m, 1H), 7.57-7.53 (m, 9H), 7.49-7.39 (m, 5H), 7.37-7.28 (m, 11H), 7.20-7.17 (m, 2H), 7.05-7.00 (m, 4H), 6.88-6.84 (m, 2H).

### Synthesis Example 27

### Synthesis of Compound 58

Compound 58 was prepared in the same manner as used to prepare Compound 53 of Synthesis Example 4, except that Intermediate 1-42 of Synthesis Example 22 was used instead of Intermediate 1-15, and Intermediate I-49 was used instead of Intermediate I-18. The formed compound was confirmed by MS/FAB.

C₅₁H₃₇N₃: calc. 691.29, found 691.38 ¹H NMR (CDCl₃, 400 MHz) δ(ppm) 8.73-8.71 (m, 2H), 7.69-7.66 (m, 2H), 7.57-7.42 (m, 10H), 7.39-7.28 (m, 11H), 7.08-7.00 (m, 5H), 6.81-6.78 (m, 2H), 6.69-6.63 (m, 3H), 6.22-6.20 (m, 2H).

### Synthesis Example 28

### Synthesis of Compound 66

Compound 66 was prepared in the same manner as used to prepare Compound 28 of Synthesis Example 2 Intermediate methylethylketone was used instead of benzylphenylketone and 2-iodo-9,9-dimethylfluorene was used instead of iodobenzene. The formed compound was confirmed by MS/FAB.

C₄₉H₃₈D₂N₂: calc. 658.33, found 658.42 ¹H NMR (CDCl₃, 400 MHz) δ(ppm) 7.87-7.85 (m, 1H), 7.78-7.76 (m, 1H), 7.66-7.60 (m, 2H), 7.58-7.51 (m, 3H), 7.48-7.45 (m, 2H), 7.43-7.31 (m, 3H), 7.21-7.18 (m, 2H), 7.13-6.99 (m, 6H), 6.79-6.76 (m, 3H), 6.66-6.63 (m, 1H), 6.25-6.22 (m, 2H), 2.40 (s, 3H), 2.33 (s, 3H), 1.64 (s, 6H).

### Synthesis Example 29

### Synthesis of Compound 71

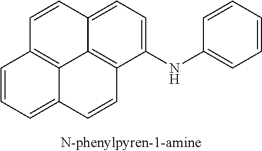 [SMILES: c1ccc(Nc2ccc3ccc4cccc5ccc2c3c45)cc1]

Compound 71 was prepared in the same manner as used to prepare Compound 28 of Synthesis Example 2, except that N-phenylpyren-1-amine was used instead of N-phenyl-2-naphthylamine, methylethylketone was used instead of benzylphenylketone, and tert-butyl iodide was used instead of iodobenzene. The formed compound was confirmed by MS/FAB.

C₄₄H₃₆D₂N₂: calc. 596.31, found 596.40 ¹H NMR (CDCl₃, 400 MHz) δ(ppm) 8.14-8.12 (m, 3H), 8.02-7.99 (m, 2H), 7.82-7.76 (m, 2H), 7.68 (d, 1H), 7.59-7.55 (m, 2H), 7.46-7.41 (m, 3H) 7.14 (dd, 1H), 7.06-7.02 (m, 2H), 6.73-6.70 (m, 2H), 6.65-6.61 (m, 1H), 6.19-6.17 (m, 2H), 2.64 (s, 3H), 2.35 (s, 3H), 1.66 (s, 9H).

### Synthesis Example 30

### Synthesis of Compound 73

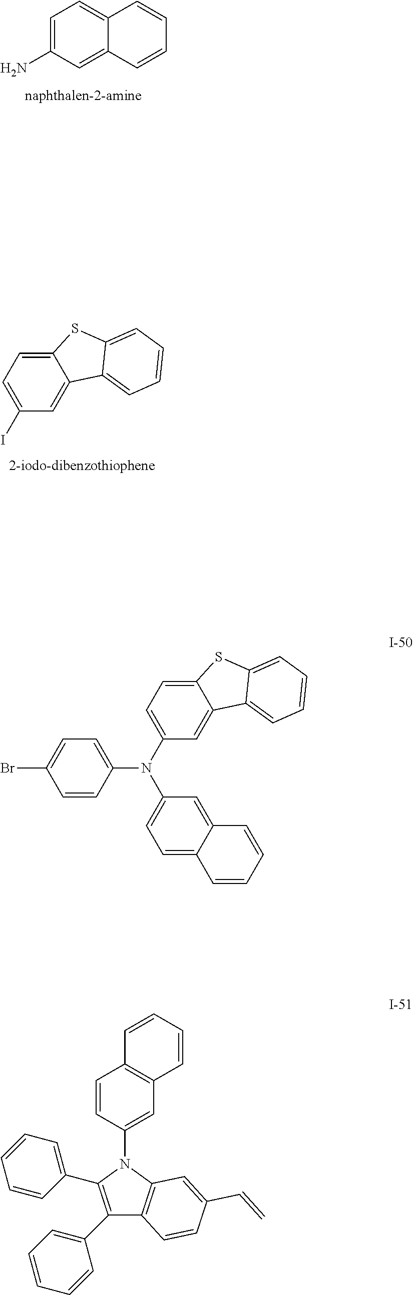 [SMILES: Nc1ccc2ccccc2c1 ; Ic1ccc2sc3ccccc3c2c1 ; Brc1ccc(N(c2ccc3ccccc3c2)c2ccc3sc4ccccc4c3c2)cc1 ; C=Cc1ccc2c(-c3ccccc3)c(-c3ccccc3)n(-c3ccc4ccccc4c3)c2c1]

Intermediate I-50 was prepared in the same manner as used to prepare Intermediate 1-2 of Synthesis Example 1, except that naphthalene-2-amine was used instead of aniline and 2-iodo-dibenzothiophene was used instead of 3-iodo-9-phenylcarbazole. Intermediate I-51 was prepared in the same manner as used to prepare Intermediate 1-5 of Synthesis Example 1, except that 3-bromostyrene was used instead of 4-bromostyrene group, benzylphenylketone was used instead of methylethylketone, and 2-iodonaphthalene was used instead of iodobenzene. Then, Compound 73 was prepared in the same manner as used to prepare Intermediate I-6 of Synthesis Example 1, except that Intermediate I-50 was used instead of Intermediate I-2, and Intermediate I-51 was used instead of Intermediate I-5. The formed compound was confirmed by MS/FAB.

C₆₀H₄₀N₂S: calc. 820.29, found 820.37 ¹H NMR (CDCl₃, 400 MHz) δ(ppm) 8.07-8.03 (m, 1H), 7.92-7.89 (m, 2H), 7.85-7.76 (m, 4H), 7.73-7.68 (m, 2H), 7.63-7.54 (m, 6H), 7.52-7.48 (m, 2H), 7.46-7.39 (m, 6H), 7.37-7.18 (m, 11H), 6.95-6.84 (m, 6H).

### Synthesis Example 31

### Synthesis of Compound 75

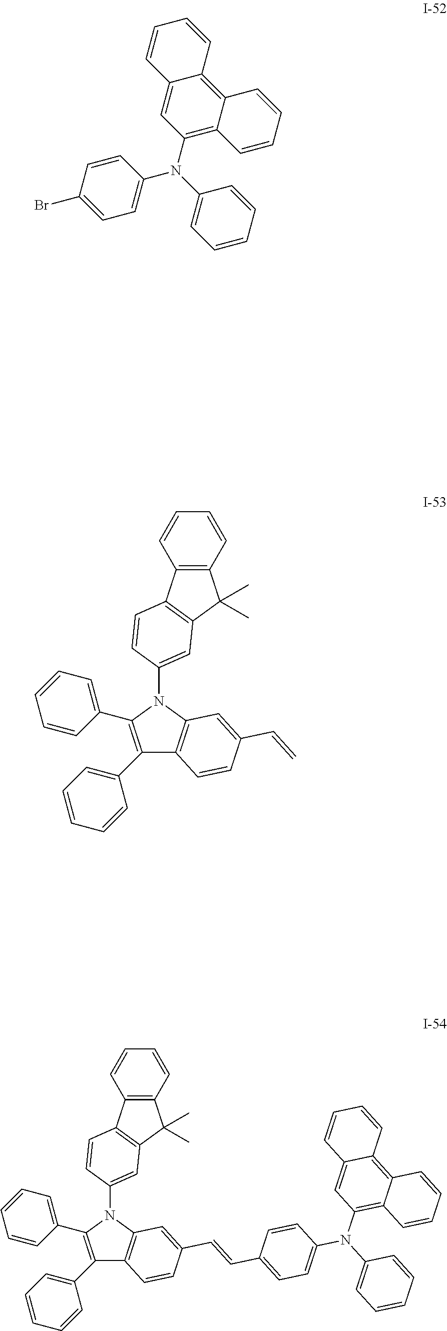 [SMILES: Brc1ccc(N(c2ccccc2)c2cc3ccccc3c3ccccc23)cc1 ; C=Cc1ccc2c(-c3ccccc3)c(-c3ccccc3)n(-c3ccc4c(c3)C(C)(C)c3ccccc3-4)c2c1 ; CC1(C)c2ccccc2-c2ccc(-n3c(-c4ccccc4)c(-c4ccccc4)c4ccc(/C=C/c5ccc(N(c6ccccc6)c6cc7ccccc7c7ccccc67)cc5)cc43)cc21]

Intermediate I-52 was prepared in the same manner as used to prepare Intermediate 1-2 of Synthesis Example 1, except that 9-bromophenanthrene was used instead of 3-iodo-9-phenyl-carbazole. Intermediate I-53 was prepared in the same manner as used to prepare Intermediate 1-5 of Synthesis Example 1, except that 3-bromostyrene was used instead of 4-bromostyrene and 2-iodo-9,9-dimethylfluorene was used instead of iodobenzene. Intermediate I-54 was prepared in the same manner as used to prepare Intermediate 1-6 of Synthesis Example 1, except that Intermediate I-52 was used instead of Intermediate 1-2, and Intermediate I-53 was used instead of Intermediate 1-5. Then, Compound 75 was prepared in the same manner as used to prepare Compound 10 of Synthesis Example 1, except that Intermediate I-54 was used instead of Intermediate I-6. The formed compound was confirmed by MS/FAB.

C₆₃H₄₄D₂N₂: calc. 832.37, found 832.45 ¹H NMR (CDCl₃, 400 MHz) δ(ppm) 8.59-8.57 (m, 1H), 8.20-8.17 (m, 1H), 7.95-7.82 (m, 4H), 7.72-7.68 (m, 2H), 7.60-7.55 (m, 3H), 7.47-7.41 (m, 5H), 7.38-7.19 (m, 7H), 7.13-6.98 (m, 7H), 6.94-6.88 (m, 2H), 6.67-6.61 (m, 4H), 6.12-6.09 (m, 2H), 1.64 (s, 6H).

### Synthesis Example 32

### Synthesis of Compound 78

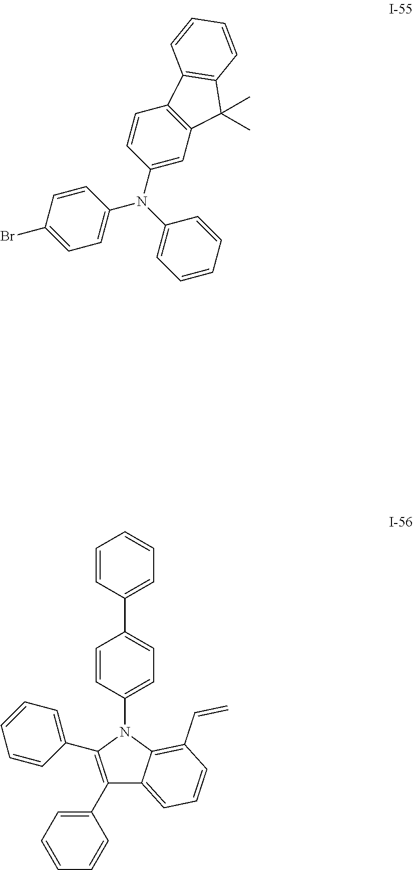 [SMILES: CC1(C)c2ccccc2-c2ccc(N(c3ccccc3)c3ccc(Br)cc3)cc21 ; C=Cc1cccc2c(-c3ccccc3)c(-c3ccccc3)n(-c3ccc(-c4ccccc4)cc3)c12]

Intermediate I-55 was prepared in the same manner as used to prepare Intermediate 1-2 of Synthesis Example 1, except that p-toluidine was used instead of aniline and 2-iodo-9,9-dimethylfluorene was used instead of 3-iodo-9-phenylcarbazole. Intermediate I-56 was prepared in the same manner as used to prepare Intermediate 1-5 of Synthesis Example 1, except that 3-bromostyrene was used instead of 4-bromostyrene and 4-iodobiphenyl was used instead of iodobenzene. Compound 78 was prepared in the same manner as used to prepare Intermediate 1-6 of Synthesis Example 1, except that Intermediate I-55 was used instead of Intermediate 1-2, and Intermediate I-56 was used instead of Intermediate 1-5. The formed compound was confirmed by MS/FAB.

C₆₂H₄₈N₂: calc. 820.38, found 820.45 ¹H NMR (CDCl₃, 400 MHz) δ(ppm) 7.78-7.76 (m, 1H), 7.69-7.61 (m, 4H), 7.56-7.29 (m, 19H), 7.12-7.09 (m, 2H), 7.05-6.96 (m, 5H), 6.92-6.89 (m, 2H), 6.81-6.78 (m, 2H), 6.71-6.68 (m, 1H), 6.56-6.52 (m, 2H), 6.42-6.41 (m, 1H), 2.29 (s, 3H), 1.61 (s, 6H).

### Synthesis Example 33

### Synthesis of Compound 79

Compound 79 was prepared in the same manner as used to prepare Compound 28 of Synthesis Example 1, except that 2-bromo-5-iodopyridine was used instead of 1-bromo-4-iodobenzene in synthesizing Intermediate I-7. The formed compound was confirmed by MS/FAB.

C₄₉H₃₃D₂N₃: calc. 667.29, found 667.37 ¹H NMR (CDCl₃, 400 MHz) δ(ppm) 8.08-8.07 (m, 1H), 7.87-7.86 (m, 1H), 7.78-7.76 (m, 1H), 7.70-7.66 (m, 2H), 7.62-7.53 (m, 5H), 7.51-7.29 (m, 14H), 7.21-7.18 (m, 1H), 7.14-7.09 (m, 2H), 7.05-7.02 (m, 2H), 6.98-6.95 (m, 1H), 6.66-6.63 (m, 1H), 6.41-6.38 (m, 2H).

### Synthesis Example 34

### Synthesis of Compound 81

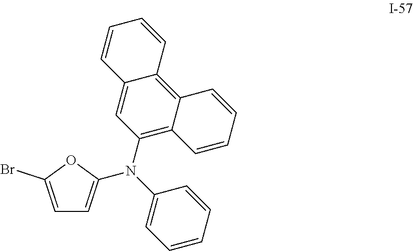 [SMILES: Brc1ccc(N(c2ccccc2)c2cc3ccccc3c3ccccc23)o1]

Intermediate I-57 was prepared in the same manner as used to prepare Intermediate I-2 of Synthesis Example 1, except that 3-bromophenanthrene was used instead of 3-iodo-9-phenyl-carbazole and 2-bromo-5-iodofuran was used instead of 1-bromo-4-iodobenzene. Then, Compound 81 was prepared in the same manner as used to prepare Compound 10 of Synthesis Example 1, except that Intermediate I-9 of Synthesis Example 2 was used instead of Intermediate I-5, and Intermediate I-57 was used instead of Intermediate I-2. The formed compound was confirmed by MS/FAB.

C₅₂H₃₄D₂N₂O: calc. 706.29, found 706.38 ¹H NMR (CDCl₃, 400 MHz) δ(ppm) 8.59-8.57 (m, 1H), 8.37-8.35 (m, 1H), 8.02-8.01 (m, 1H), 7.71-7.69 (m, 2H), 7.60-7.27 (m, 20H), 7.14-7.10 (m, 2H), 7.04-7.00 (m, 2H), 6.78-6.73 (m, 2H), 6.47-6.45 (m, 2H), 5.02-5.01 (m, 1H).

### Synthesis Example 35

### Synthesis of Compound 83

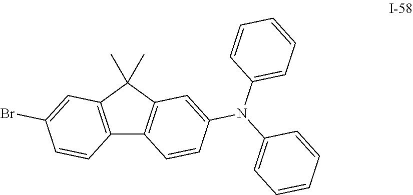 [SMILES: CC1(C)c2cc(Br)ccc2-c2ccc(N(c3ccccc3)c3ccccc3)cc21]

Intermediate I-58 was prepared in the same manner as used to prepare Intermediate I-2 of Synthesis Example 1, except that iodobenzene was used instead of 3-iodo-9-phenyl-carbazole and 2,7-diiodo-9,9-dimethylfluorene was used instead of 1-bromo-4-iodobenzene. Then, Compound 83 was prepared in the same manner as used to prepare Compound 10 of Synthesis Example 1, except that Intermediate I-9 of Synthesis Example 2 was used instead of Intermediate I-5, and Intermediate I-58 was used instead of Intermediate I-2. The formed compound was confirmed by MS/FAB.

C₅₅H₄₀D₂N₂: calc. 732.34, found 732.44 ¹H NMR (CDCl₃, 400 MHz) δ(ppm) 7.78-7.77 (m, 1H), 7.76-7.73 (m, 1H), 7.68-7.66 (m, 1H), 7.65-7.63 (m, 1H), 7.62-7.60 (m, 1H), 7.59-7.56 (m, 3H), 7.49-7.29 (m, 11H), 7.09-7.02 (m, 6H), 6.67-6.62 (m, 4H), 6.46-6.45 (m, 1H), 6.16-6.13 (m, 4H), 1.61 (s, 6H).

### Example 1

As an anode, a 15 Ω/cm² (1200 Å) ITO glass substrate manufactured by Corning Co., Ltd was cut to a size of 50 mm×50 mm×0.7 mm and sonicated with isopropyl alcohol and pure water each for 5 minutes, and then a ultraviolet ray was irradiated thereto for 30 minutes, followed by exposure to ozone. 2-TNATA was vacuum deposited on the glass substrate to form an HIL having a thickness of 600 Å, and then, 4,4′-bis[N-(1-naphthyl)-N-phenylamino]biphenyl (NPB) was vacuum deposited on the HIL to form a hole transport layer having a thickness of 300 Å. 98 wt % of ADN as a blue fluorescent host and 2 wt % of Compound 10 as a blue fluorescent dopant were used on the HTL to form an EML having a thickness of 300 Å. Alq₃ was vacuum deposited on the EML to form an ETL having a thickness of 300 Å. LiF was vacuum deposited on the ETL to form an EIL having a thickness of 10 Å and then Al was vacuum deposited thereon to form a cathode having a thickness of 3000 Å, thereby completing manufacture of an organic light-emitting diode.

### Example 2

An organic light-emitting diode was manufactured in the same manner as in Example 1, except that Compound 28 was used instead of Compound 10 as a dopant in forming the EML.

### Example 3

An organic light-emitting diode was manufactured in the same manner as in Example 1, except that Compound 35 was used instead of Compound 10 as a dopant in forming the EML.

### Example 4

An organic light-emitting diode was manufactured in the same manner as in Example 1, except that Compound 53 was used instead of Compound 10 as a dopant in forming the EML.

### Example 5

An organic light-emitting diode was manufactured in the same manner as in Example 1, except that Compound 67 was used instead of Compound 10 as a dopant in forming the EML.

### Example 6

An organic light-emitting diode was manufactured in the same manner as in Example 1, except that Compound 77 was used instead of Compound 10 as a dopant in forming the EML.

### Example 7

An organic light-emitting diode was manufactured in the same manner as in Example 1, except that Compound 83 was used instead of Compound 10 as a dopant in forming the EML.

### Comparative Example 1

An organic light-emitting diode was manufactured in the same manner as in Example 1, except that DPAVBi was used instead of Compound 10 as a dopant in forming the EML.

### Evaluation Example

The driving voltage, current density, brightness, efficiency, emission color, half lifetime of the organic light-emitting diodes manufactured according to Examples 1 to 7 and Comparative Example 1 were evaluated by using a PR650 Spectroscan Source Measurement Unit. (product of PhotoResearch Co., Ltd). Results thereof are shown in Table 1 below:

TABLE 1

| | | | Driving | Current | | | | |
|---|---|---|---|---|---|---|---|---|
| | EML | | voltage | density | Brightness | Efficiency | Emission | Lifetime |
| | host | EMLdopant | (V) | (mA/cm²) | (cd/m²) | (cd/A) | color | (hr)¹ |
| Example 1 | ADN | Compound | 6.23 | 50 | 2,910 | 5.82 | Blue | 234 hr |
| | | 10 | | | | | | |
| Example 2 | ADN | Compound | 6.27 | 50 | 3,045 | 6.09 | Blue | 277 hr |
| | | 28 | | | | | | |
| Example 3 | ADN | Compound | 6.35 | 50 | 3,075 | 6.15 | Blue | 269 hr |
| | | 35 | | | | | | |
| Example 4 | ADN | Compound | 6.40 | 50 | 3,255 | 6.51 | Blue | 289 hr |
| | | 53 | | | | | | |
| Example 5 | ADN | Compound | 5.95 | 50 | 3,170 | 6.34 | Blue | 219 hr |
| | | 67 | | | | | | |
| Example 6 | ADN | Compound | 6.29 | 50 | 2,850 | 5.70 | Blue | 256 hr |
| | | 77 | | | | | | |
| Example 7 | ADN | Compound | 6.13 | 50 | 3,320 | 6.64 | Blue | 323 hr |
| | | 83 | | | | | | |
| Comparative | ADN | DPAVBi | 7.35 | 50 | 2,065 | 4.13 | Blue | 145 hr |
| Example1 | | | | | | | | |

¹Reference current density corresponding to half lifetime: 100 mA/cm²

Referring to Table 2, it was confirmed that the organic light-emitting diodes manufactured according to Examples 1 to Example 7 show excellent performances than the organic light-emitting diode manufactured according to Comparative Example 1 in terms of a driving voltage, brightness, efficiency, and lifetime.

Organic light-emitting diodes including the heterocyclic compounds described above may show excellent performances, for example, low driving voltage, high brightness, high efficiency, and a long lifetime.

While the present invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. A heterocyclic compound represented by Formula 1A below: 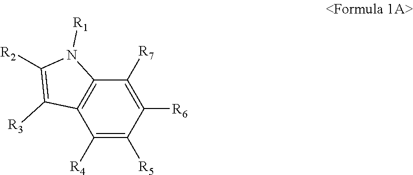
in Formula 1A,
R₁ to R₇ are each independently a hydrogen atom, a deuterium atom, a halogen atom, a hydroxyl group, a cyano group, a nitro group, a carboxyl group, a substituted or unsubstituted C₁-C₃₀ alkyl group, a substituted or unsubstituted C₂-C₃₀ alkenyl group, a substituted or unsubstituted C₂-C₃₀ alkynyl group, a substituted or unsubstituted C₁-C₃₀ alkoxy group, a substituted or unsubstituted C₃-C₃₀ cycloalkyl group, a substituted or unsubstituted C₃-C₃₀ cycloalkenyl group, a substituted or unsubstituted C₅-C₃₀ aryl group, a substituted or unsubstituted C₂-C₃₀ heteroaryl group, a substituted or unsubstituted C₅-C₃₀ aryloxy group, a substituted or unsubstituted C₅-C₃₀ arylthio group, a group represented by N(Q₁)(Q₂), or a group represented by Formula 1B below, wherein Q₁ and Q₂ are each independently a hydrogen atom, a deuterium atom, a substituted or unsubstituted C₁-C₃₀ alkyl group, a substituted or unsubstituted C₂-C₃₀ alkenyl group, a substituted or unsubstituted C₂-C₃₀ alkynyl group, a substituted or unsubstituted C₁-C₃₀ alkoxy group, a substituted or unsubstituted C₃-C₃₀ cycloalkyl group, a substituted or unsubstituted C₃-C₃₀ cycloalkenyl group, a substituted or unsubstituted C₆-C₃₀ aryl group, a substituted or unsubstituted C₃-C₃₀ heteroaryl group, a substituted or unsubstituted C₆-C₃₀ aryloxy group, or a substituted or unsubstituted C₆-C₃₀ arylthio group; 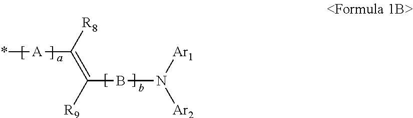
in Formula 1B,
R₉ and R₉ are each independently a hydrogen atom, a deuterium atom, a halogen atom, a hydroxyl group, a cyano group, a nitro group, a carboxyl group, a substituted or unsubstituted C₁-C₃₀ alkyl group, a substituted or unsubstituted C₂-C₃₀ alkenyl group, a substituted or unsubstituted C₂-C₃₀ alkynyl group, a substituted or unsubstituted C₁-C₃₀ alkoxy group, a substituted or unsubstituted C₃-C₃₀ cycloalkyl group, a substituted or unsubstituted C₃-C₃₀ cycloalkenyl group, a substituted or unsubstituted C₅-C₃₀ aryl group, a substituted or unsubstituted C₂-C₃₀ heteroaryl group, a substituted or unsubstituted C₅-C₃₀ aryloxy group, or a substituted or unsubstituted C₅-C₃₀ arylthio group;
at least one of R₁ to R₇ is a group represented by Formula 1B above;
Ar₁ and Ar₂ are each independently a substituted or unsubstituted C₅-C₃₀ aryl group, or a substituted or unsubstituted C₃-C₃₀ heteroaryl group;
A and B are each a bivalent linker and are each independently a substituted or unsubstituted C₅-C₃₀ arylene group, or a substituted or unsubstituted C₃-C₃₀ heteroarylene group; and
a is an integer of 0 to 3, and if a is 2 or more, 2 or more A are identical to or different from each other, and b is an integer of 0 to 3, and if b is 2 or more, 2 or more B are identical to or different from each other, and * is a binding site.

2. The heterocyclic compound of claim 1, wherein Ar₁ and Ar₂ are each independently a hydrogen atom, a deuterium atom, a halogen atom, a hydroxyl group, a cyano group, a nitro group, a carboxyl group, a substituted or unsubstituted methyl group, a substituted or unsubstituted ethyl group, a substituted or unsubstituted propyl group, a substituted or unsubstituted butyl group, a substituted or unsubstituted pentyl group, a substituted or unsubstituted ethenyl group, a substituted or unsubstituted phenyl group, a substituted or unsubstituted biphenyl group, a substituted or unsubstituted terphenyl group, a substituted or unsubstituted naphthyl group, a substituted or unsubstituted phenoxy, a substituted or unsubstituted fluorenyl group, a substituted or unsubstituted spiro-fluorenyl group, a substituted or unsubstituted carbazolyl group, a substituted or unsubstituted phenanthrenyl group, a substituted or unsubstituted pyranyl group, a substituted or unsubstituted pyridinyl group, a substituted or unsubstituted pyrimidinyl group, a substituted or unsubstituted diazinyl group, a substituted or unsubstituted triazinyl group, a substituted or unsubstituted quinolinyl group, a substituted or unsubstituted benzoimidazolyl group, a substituted or unsubstituted benzoxazolyl group, a substituted or unsubstituted pentalenyl group, a substituted or unsubstituted indenyl group, a substituted or unsubstituted azulenyl group, a substituted or unsubstituted heptalenyl group, a substituted or unsubstituted indacenyl group, a substituted or unsubstituted acenaphthyl group, a substituted or unsubstituted phenalenyl group, a substituted or unsubstituted phenanthridinyl group, a substituted or unsubstituted anthryl group, a substituted or unsubstituted fluoranthenyl group, a substituted or unsubstituted triphenylenyl group, a substituted or unsubstituted pyrenyl group, a substituted or unsubstituted chrysenyl group, a substituted or unsubstituted naphthacenyl group, a substituted or unsubstituted picenyl group, a substituted or unsubstituted perylenyl group, a substituted or unsubstituted pentaphenyl group, a substituted or unsubstituted hexacenyl group, a substituted or unsubstituted pyrrolyl group, a substituted or unsubstituted imidazolyl group, a substituted or unsubstituted pyrazolyl group, a substituted or unsubstituted imidazopyridinyl group, a substituted or unsubstituted pyrazinyl group, a substituted or unsubstituted imidazopyrimidinyl group, a substituted or unsubstituted pyridazinyl group, a substituted or unsubstituted indolyl group, a substituted or unsubstituted indolizinyl group, a substituted or unsubstituted isoindolyl group, a substituted or unsubstituted pyridoindolyl group, a substituted or unsubstituted indazolyl group, a substituted or unsubstituted carbazolyl group, a substituted or unsubstituted purinyl group, a substituted or unsubstituted benzoquinolinyl group, a substituted or unsubstituted phthalazinyl group, a substituted or unsubstituted naphthyridinyl group, a substituted or unsubstituted quinoxalinyl group, a substituted or unsubstituted quinazolinyl group, a substituted or unsubstituted cinnolinyl group, a substituted or unsubstituted phenazinyl group, a substituted or unsubstituted furanyl group, a substituted or unsubstituted benzofuranyl group, a substituted or unsubstituted dibenzofuranyl group, a substituted or unsubstituted thiophenyl group, a substituted or unsubstituted benzothiophenyl group, a substituted or unsubstituted dibenzothiophenyl group, a substituted or unsubstituted thiazolyl group, a substituted or unsubstituted isothiazolyl group, a substituted or unsubstituted benzothiazolyl group, a substituted or unsubstituted oxazolyl group, a substituted or unsubstituted isooxazolyl group, a substituted or unsubstituted oxadiazolyl group, a substituted or unsubstituted triazolyl group, or a substituted or unsubstituted tetrazolyl group.

3. The heterocyclic compound of claim 1, wherein Ar₁ and Ar₂ are each independently one of groups represented by Formulae 2A to 2I below: 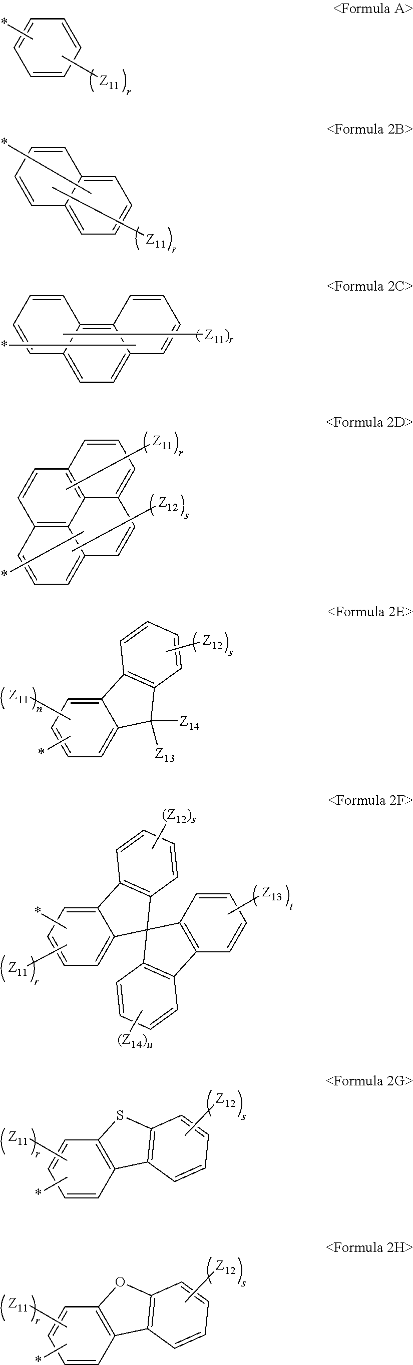 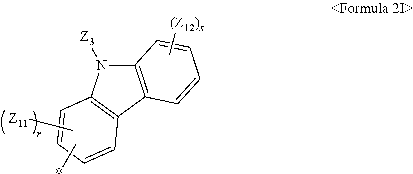
in Formula 2A to 2I,
Z₁₁, Z₁₂, Z₁₃ and Z₁₄ are each independently a hydrogen atom, a deuterium atom, a halogen atom, a hydroxyl group, a cyano group, a substituted or unsubstituted methyl group, a substituted or unsubstituted ethyl group, a substituted or unsubstituted propyl group, a substituted or unsubstituted butyl group, a substituted or unsubstituted methoxy group, a substituted or unsubstituted ethoxy group, a substituted or unsubstituted phenyl group, a substituted or unsubstituted naphthyl group, a substituted or unsubstituted anthryl group, a substituted or unsubstituted pyridinyl group, or a substituted or unsubstituted quinolinyl group, and
a plurality of each of Z₁₁, Z₁₂, Z₁₃ and Z₁₄ are identical to or different from each other, r is an integer of 1 to 9, s, t and u are each an integer of 1 to 4, and * indicates a binding site.

4. The heterocyclic compound of claim 3, wherein Ar₁ and Ar₂ are each independently one of groups represented by Formulae 3A to 3Q below: 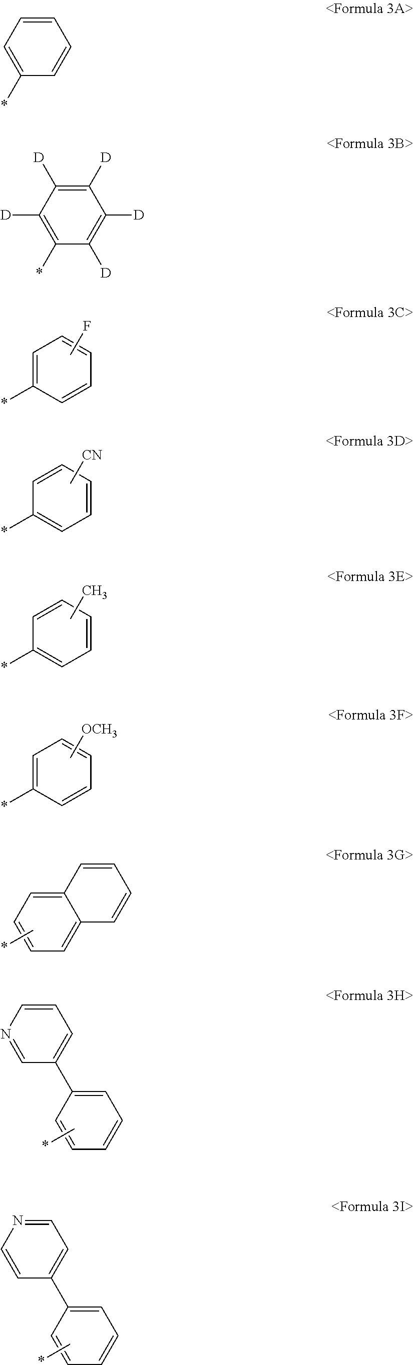 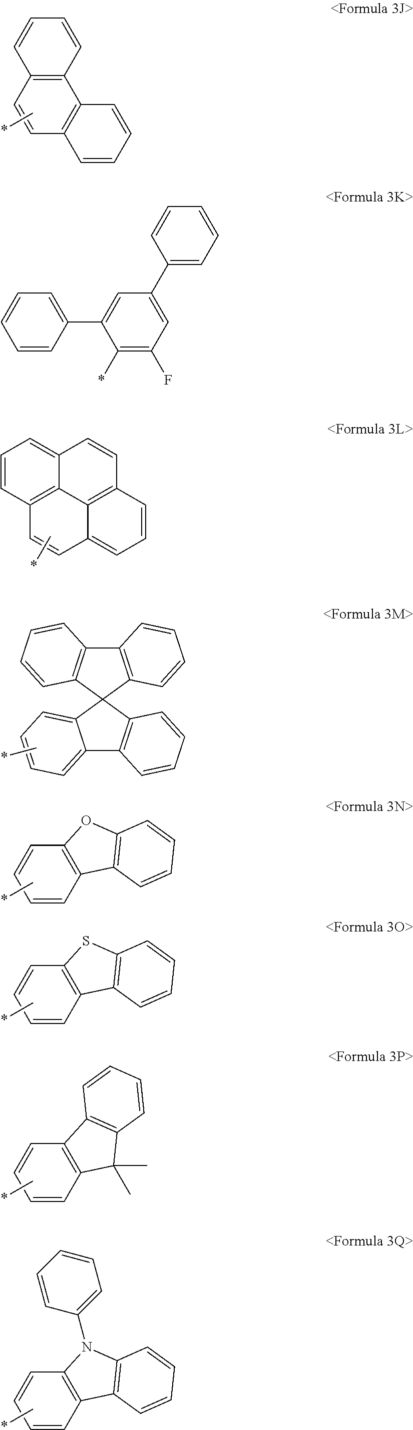
in Formulae 3A to 3Q, * indicates a binding site.

5. The heterocyclic compound of claim 1, wherein A and B are each independently a substituted or unsubstituted phenylene group, a substituted or unsubstituted pentalenylene group, a substituted or unsubstituted indenylene group, a substituted or unsubstituted naphthylene group, a substituted or unsubstituted azulenylene group, a substituted or unsubstituted heptalenylene group, a substituted or unsubstituted indacenylene group, a substituted or unsubstituted acenaphthylene group, a substituted or unsubstituted fluorenylene group, a substituted or unsubstituted phenalenylene group, a substituted or unsubstituted phenanthrenylene group, a substituted or unsubstituted anthrylene group, a substituted or unsubstituted fluoranthenylene group, a substituted or unsubstituted triphenylenylene group, a substituted or unsubstituted pyrenylene group, a substituted or unsubstituted chrysenylene group, a substituted or unsubstituted naphthacenylene group, a substituted or unsubstituted picenylene group, a substituted or unsubstituted perylenylene group, a substituted or unsubstituted pentaphenylene group, a substituted or unsubstituted hexacenylene group, a substituted or unsubstituted pyrrolylene group, a substituted or unsubstituted pyrazolylene group, a substituted or unsubstituted imidazolylene group, a substituted or unsubstituted imidazolinylene group, a substituted or unsubstituted imidazopyridinylene group, a substituted or unsubstituted imidazopyrimidinylene group, a substituted or unsubstituted pyridinylene group, a substituted or unsubstituted pyrazinylene group, a substituted or unsubstituted pyrimidinylene group, a substituted or unsubstituted indolylene group, a substituted or unsubstituted purinylene group, a substituted or unsubstituted quinolinylene group, a substituted or unsubstituted phthalazinylene group, a substituted or unsubstituted indolizinylene group, a substituted or unsubstituted naphthyridinylene group, a substituted or unsubstituted quinazolinylene group, a substituted or unsubstituted cinnolinylene group, a substituted or unsubstituted indazolylene group, a substituted or unsubstituted carbazolylene group, a substituted or unsubstituted phenazinylene group, a substituted or unsubstituted phenanthridinylene group, a substituted or unsubstituted pyrenylene group, a substituted or unsubstituted chromenylene group, a substituted or unsubstituted furanylene group, a substituted or unsubstituted benzofuranylene group, a substituted or unsubstituted thiophenylene group, a substituted or unsubstituted benzothiophenylene group, a substituted or unsubstituted isothiazolylene group, a substituted or unsubstituted benzoimidazolylene group, a substituted or unsubstituted isoxazolylene group, a substituted or unsubstituted dibenzothiophenylene group, a substituted or unsubstituted dibenzofuranylene group, a substituted or unsubstituted triazinylene group, or a substituted or unsubstituted oxadiazolylene group.

6. The heterocyclic compound of claim 1, wherein the A and B are each independently a substituted or unsubstituted phenylene group, a substituted or unsubstituted naphthylene group, a substituted or unsubstituted fluorenyl group, a substituted or unsubstituted carbazolylene group, a substituted or unsubstituted dibenzothiophenylene group, a substituted or unsubstituted pyridinylene group, a substituted or unsubstituted pyrimidinylene group, a substituted or unsubstituted triazinylene group, a substituted or unsubstituted anthrylene group, a substituted or unsubstituted phenanthrenylene group, a substituted or unsubstituted pyrenylene group, a substituted or unsubstituted chrycenyl group, a substituted or unsubstituted perylenylene group, a substituted or unsubstituted spiro-fluorenyl group, a substituted or unsubstituted furanylene group, a substituted or unsubstituted thiophenylene group, or a substituted or unsubstituted oxadiazolylene group.

7. The heterocyclic compound of claim 1, wherein A and B are each independently one of groups represented by Formulae 4A to 4E: 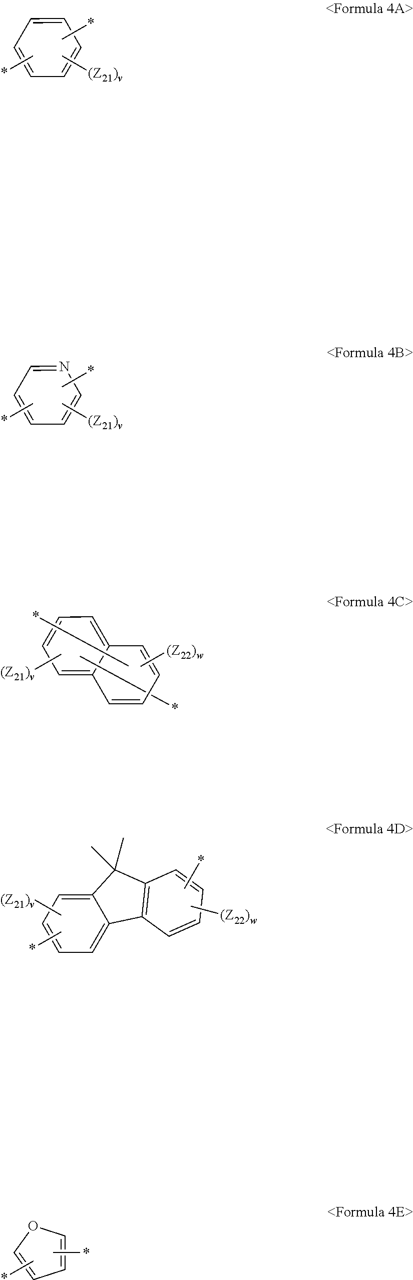
in Formulae 4A to 4E,
Z₂₁ and Z₂₂ are each independently a hydrogen atom, a deuterium atom, a halogen atom, a hydroxyl group, a cyano group, a substituted or unsubstituted methyl group, a substituted or unsubstituted ethyl group, a substituted or unsubstituted propyl group, a substituted or unsubstituted butyl group, a substituted or unsubstituted methoxy group, a substituted or unsubstituted ethoxy group, a substituted or unsubstituted phenyl group, a substituted or unsubstituted naphthyl group, or a substituted or unsubstituted pyridinyl group, a plurality of each of Z₂₁ and Z₂₂ are identical to or different from each other, v and w are each an integer of 1 to 4, * and *′ indicate a binding site.

8. The heterocyclic compound of claim 1, wherein A and B are each independently one of groups represented by Formulae 5A to 5F below:

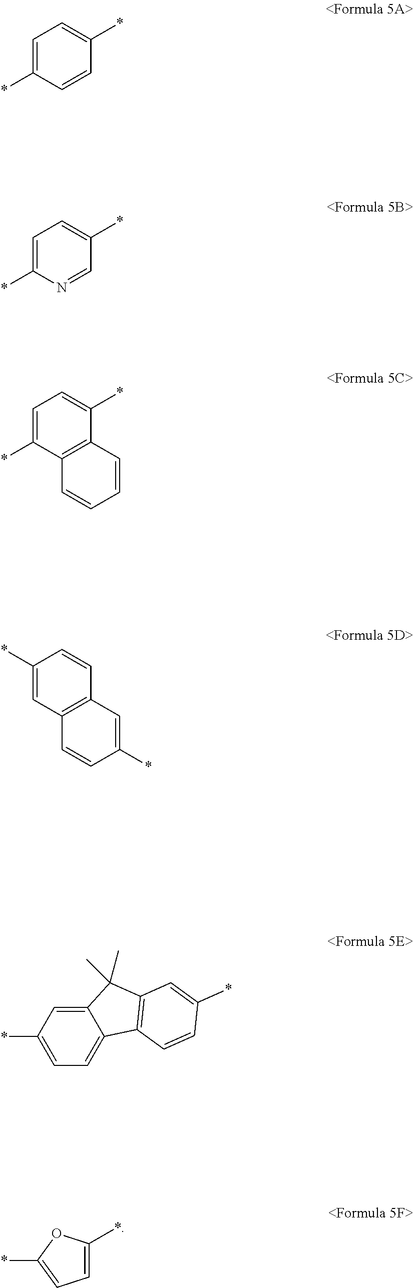

9. The heterocyclic compound of claim 1, wherein R₁ to R₇ are each independently a hydrogen atom, a deuterium atom, a halogen atom, a hydroxyl group, a cyano group, a nitro group, a carboxyl group, a substituted or unsubstituted C₁-C₂₀ alkyl group, a substituted or unsubstituted C₁-C₂₀ alkoxy group, a substituted or unsubstituted phenyl group, a substituted or unsubstituted pentalenyl group, a substituted or unsubstituted indenyl group, a substituted or unsubstituted naphthyl group, a substituted or unsubstituted azulenyl group, a substituted or unsubstituted heptalenyl group, a substituted or unsubstituted indacenyl group, a substituted or unsubstituted acenaphthyl group, a substituted or unsubstituted fluorenyl group, a substituted or unsubstituted spiro-fluorenyl group, a substituted or unsubstituted phenalenyl group, a substituted or unsubstituted phenanthrenyl group, a substituted or unsubstituted anthryl group, a substituted or unsubstituted fluoranthenyl group, a substituted or unsubstituted triphenylenyl group, a substituted or unsubstituted pyrenyl group, a substituted or unsubstituted chrysenyl group, a substituted or unsubstituted naphthacenyl. substituted or unsubstituted picenyl group, a substituted or unsubstituted perylenyl group, a substituted or unsubstituted pentaphenyl group, a substituted or unsubstituted hexacenyl group, a substituted or unsubstituted pyrrolyl group, a substituted or unsubstituted imidazolyl group, a substituted or unsubstituted pyrazolyl group, a substituted or unsubstituted pyridinyl group, a substituted or unsubstituted pyrazinyl group, a substituted or unsubstituted pyrimidinyl group, a substituted or unsubstituted pyridazinyl group, a substituted or unsubstituted isoindolyl, a substituted or unsubstituted indolyl group, a substituted or unsubstituted indazolyl group, a substituted or unsubstituted purinyl group, a substituted or unsubstituted quinolinyl group, a substituted or unsubstituted benzoquinolinyl group, a substituted or unsubstituted phthalazinyl group, a substituted or unsubstituted naphthyridinyl group, a substituted or unsubstituted quinoxalinyl group, a substituted or unsubstituted quinazolinyl group, a substituted or unsubstituted cinnolinyl group, a substituted or unsubstituted carbazolyl group, a substituted or unsubstituted phenanthridinyl group, a substituted or unsubstituted acridinyl group, a substituted or unsubstituted phenanthrolinyl group, a substituted or unsubstituted phenazinyl group, a substituted or unsubstituted benzoxazolyl group, a substituted or unsubstituted benzoimidazolyl group, a substituted or unsubstituted furanyl group, a substituted or unsubstituted benzofuranyl group, a substituted or unsubstituted thiophenyl group, a substituted or unsubstituted benzothiophenyl group, a substituted or unsubstituted thiazolyl group, a substituted or unsubstituted isothiazolyl group, a substituted or unsubstituted benzothiazolyl group, a substituted or unsubstituted isooxazolyl group, a substituted or unsubstituted oxazolyl group, a substituted or unsubstituted triazolyl group, a substituted or unsubstituted tetrazolyl group, a substituted or unsubstituted oxadiazolyl group, a substituted or unsubstituted triazinyl group, a substituted or unsubstituted benzoxazolyl group, a substituted or unsubstituted dibenzofuranyl group, or a substituted or unsubstituted dibenzothiophenyl or benzocarbazolyl.

10. The heterocyclic compound of claim 1, wherein R₁ to R₇ are each independently a hydrogen atom, a deuterium atom, a halogen atom, a hydroxyl group, a cyano group, a nitro group, a carboxyl group, a substituted or unsubstituted methyl group, a substituted or unsubstituted ethyl group, a substituted or unsubstituted propyl group, a substituted or unsubstituted butyl group, a substituted or unsubstituted pentyl group, a substituted or unsubstituted ethenyl group, a substituted or unsubstituted phenyl group, a substituted or unsubstituted biphenyl group, a substituted or unsubstituted terphenyl group, a substituted or unsubstituted naphthyl group, a substituted or unsubstituted anthryl group, a substituted or unsubstituted phenanthrenyl group, a substituted or unsubstituted pyrenyl group, a substituted or unsubstituted fluorenyl group, a substituted or unsubstituted carbazolyl group, a substituted or unsubstituted pyranyl group, a substituted or unsubstituted pyridinyl group, a substituted or unsubstituted pyrimidinyl group, a substituted or unsubstituted pyrazinyl group, a substituted or unsubstituted pyridazinyl group, a substituted or unsubstituted triazinyl group, a substituted or unsubstituted quinolinyl group, a substituted or unsubstituted benzoimidazolyl group, a substituted or unsubstituted benzoxazolyl group, a substituted or unsubstituted pentalenyl group, a substituted or unsubstituted indenyl group, a substituted or unsubstituted pyrrolyl group, a substituted or unsubstituted imidazolyl group, a substituted or unsubstituted pyrazolyl group, a group represented by N(Q₁)(Q₂), or one of the groups represented by Formula 1B;
Q₁ and Q₂ are each independently a hydrogen atom, a deuterium atom, a halogen atom, a hydroxyl group, a carboxyl group, a substituted or unsubstituted methyl group, a substituted or unsubstituted ethyl group, a substituted or unsubstituted propyl group, a substituted or unsubstituted butyl group, a substituted or unsubstituted pentyl group, a substituted or unsubstituted phenyl group, a substituted or unsubstituted naphthyl group, a substituted or unsubstituted anthryl group, a substituted or unsubstituted fluorenyl group, a substituted or unsubstituted carbazolyl, or a substituted or unsubstituted pyridinyl;
R₈ and R₉ are each independently a hydrogen atom, a deuterium atom, a halogen atom, a hydroxyl group, a cyano group, a substituted or unsubstituted methyl group, a substituted or unsubstituted ethyl group, a substituted or unsubstituted propyl group, a substituted or unsubstituted butyl group, a substituted or unsubstituted pentyl group, a substituted or unsubstituted ethenyl group, a substituted or unsubstituted phenyl group, a substituted or unsubstituted naphthyl group, a substituted or unsubstituted anthryl group, or a substituted or unsubstituted phenanthrenyl group;
Ar₁ and Ar₂ are each independently a substituted or unsubstituted phenyl group, a substituted or unsubstituted naphthyl group, a substituted or unsubstituted phenanthrenyl group, a substituted or unsubstituted pyrenyl group, a substituted or unsubstituted anthryl group, a substituted or unsubstituted fluorenyl group, a substituted or unsubstituted spiro-fluorenyl group, a substituted or unsubstituted dibenzothiophenyl group, a substituted or unsubstituted dibenzofuranyl group, a substituted or unsubstituted carbazolyl group, a substituted or unsubstituted pyridinyl group, a substituted or unsubstituted pyrimidinyl group, a substituted or unsubstituted triazinyl group, a substituted or unsubstituted chrysenyl group, a substituted or unsubstituted perylenyl group, or a substituted or unsubstituted oxadiazolyl group;
A and B are each independently a substituted or unsubstituted phenylene group, a substituted or unsubstituted naphthylene group, a substituted or unsubstituted fluorenyl group, a substituted or unsubstituted carbazolylene group, a substituted or unsubstituted dibenzothiophenylene group, a substituted or unsubstituted pyridinylene group, a substituted or unsubstituted pyrimidinylene group, a substituted or unsubstituted triazinylene group, a substituted or unsubstituted anthrylene group, a substituted or unsubstituted phenanthrenylene group, a substituted or unsubstituted pyrenylene group, a substituted or unsubstituted chrycenyl group, a substituted or unsubstituted perylenylene group, a substituted or unsubstituted spiro-fluorenyl group, a substituted or unsubstituted furanylene group, a substituted or unsubstituted thiophenylene group, or a substituted or unsubstituted oxadiazolylene group; and
a is an integer of 0 to 2, and if a is 2, two A are identical to or different from each other, and b is an integer of 0 to 2, and if b is 2, two B are identical to or different from each other.

11. The heterocyclic compound of claim 10, wherein R₁ to R₇ are each independently a hydrogen atom, a deuterium atom, a halogen atom, a substituted or unsubstituted methyl group, a substituted or unsubstituted ethyl group, a substituted or unsubstituted propyl group, a substituted or unsubstituted butyl group, the group represented by Formula 1B, or one of groups represented by Formulae 6A to 6D below;
R₈ and R₉ are each independently a hydrogen atom, a deuterium atom, a halogen atom, a substituted or unsubstituted methyl group, a substituted or unsubstituted ethyl group, a substituted or unsubstituted propyl group, a substituted or unsubstituted butyl group, or one of groups represented by Formula 6A to 6D below: 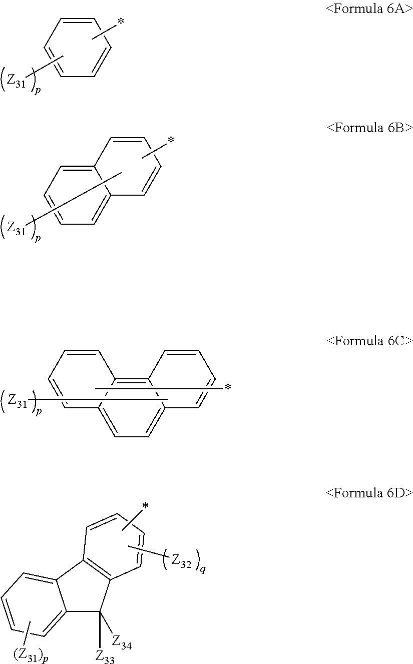
in the formulae above,
Z₃₁, Z₃₂, Z₃₃ and Z₃₄ are each independently a hydrogen atom, a deuterium atom, a halogen atom, a hydroxyl group, a cyano group, a substituted or unsubstituted methyl group, a substituted or unsubstituted ethyl group, a substituted or unsubstituted propyl group, a substituted or unsubstituted butyl group, a substituted or unsubstituted methoxy group, a substituted or unsubstituted ethoxy group, a substituted or unsubstituted ethenyl group, a substituted or unsubstituted phenyl group, a substituted or unsubstituted naphthyl group, a substituted or unsubstituted anthryl group, a substituted or unsubstituted pyridinyl group, a substituted or unsubstituted carbazolyl group, or a substituted or unsubstituted quinolinyl group, a plurality of each of Z₃₁ and Z₃₂ are identical to or different from each other, p is an integer of 1 to 9, q is an integer of 1 to 4, and * indicates a binding site.

12. The heterocyclic compound of claim 11, wherein R₁ to R₇ are each independently a hydrogen atom, a deuterium atom, a halogen atom, a substituted or unsubstituted methyl group, a substituted or unsubstituted ethyl group, a substituted or unsubstituted propyl group, a substituted or unsubstituted tertbutyl group, a cyano group, —CD₃, —CF₃, or one of groups represented by Formulae 7A to 7G,
R₈ and R₉ are each independently a hydrogen atom, a deuterium atom, or one of groups represented by Formulae 7A to 7G: 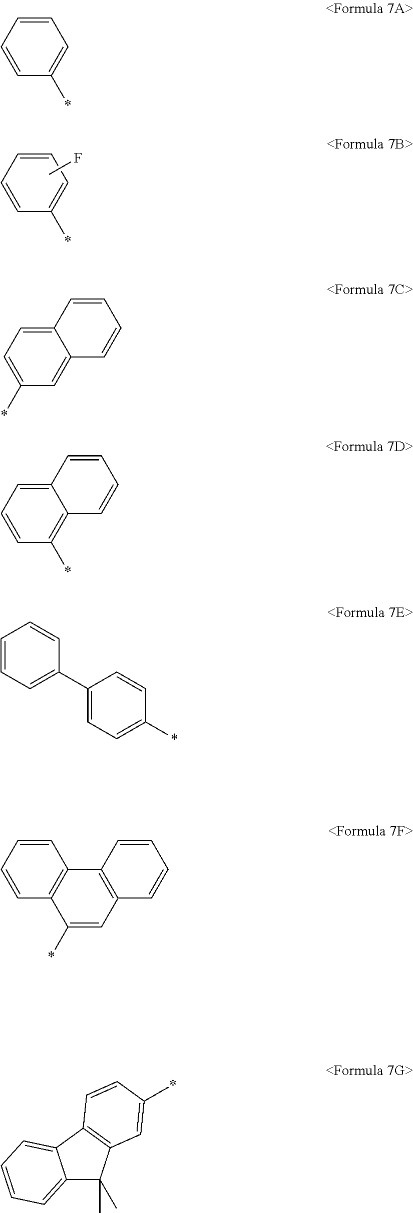
in Formulae 7A to 7G, * indicates a binding site.

13. The heterocyclic compound of claim 1, wherein the hetero compound represented by Formula 1A is one of Compound 1 to 83 illustrated below:

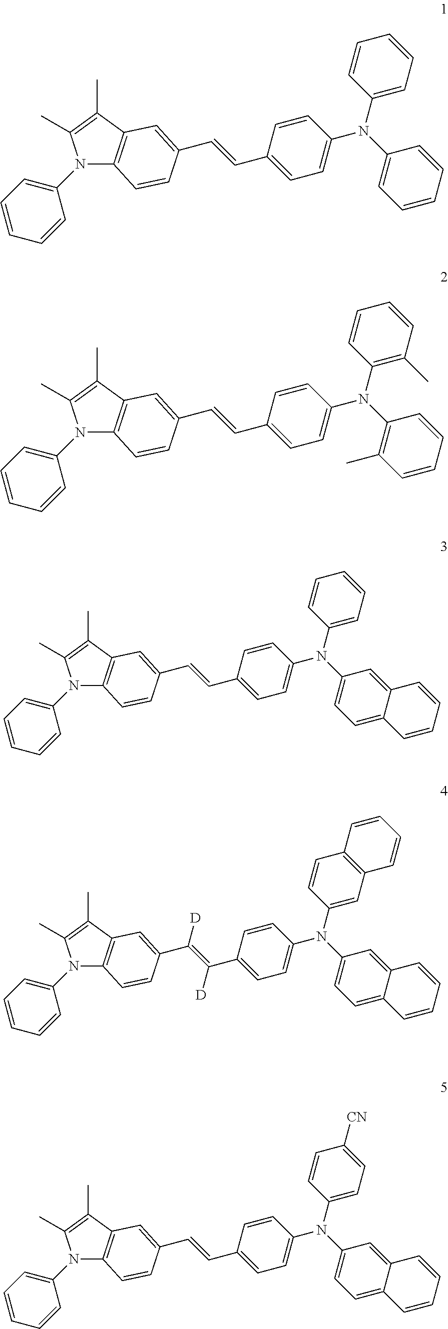 [SMILES: Cc1c(C)n(-c2ccccc2)c2ccc(/C=C/c3ccc(N(c4ccccc4)c4ccccc4)cc3)cc12 ; Cc1ccccc1N(c1ccc(/C=C/c2ccc3c(c2)c(C)c(C)n3-c2ccccc2)cc1)c1ccccc1C ; Cc1c(C)n(-c2ccccc2)c2ccc(/C=C/c3ccc(N(c4ccccc4)c4ccc5ccccc5c4)cc3)cc12 ; [2H]/C(=C(/[2H])c1ccc2c(c1)c(C)c(C)n2-c1ccccc1)c1ccc(N(c2ccc3ccccc3c2)c2ccc3ccccc3c2)cc1 ; Cc1c(C)n(-c2ccccc2)c2ccc(/C=C/c3ccc(N(c4ccc(C#N)cc4)c4ccc5ccccc5c4)cc3)cc12]

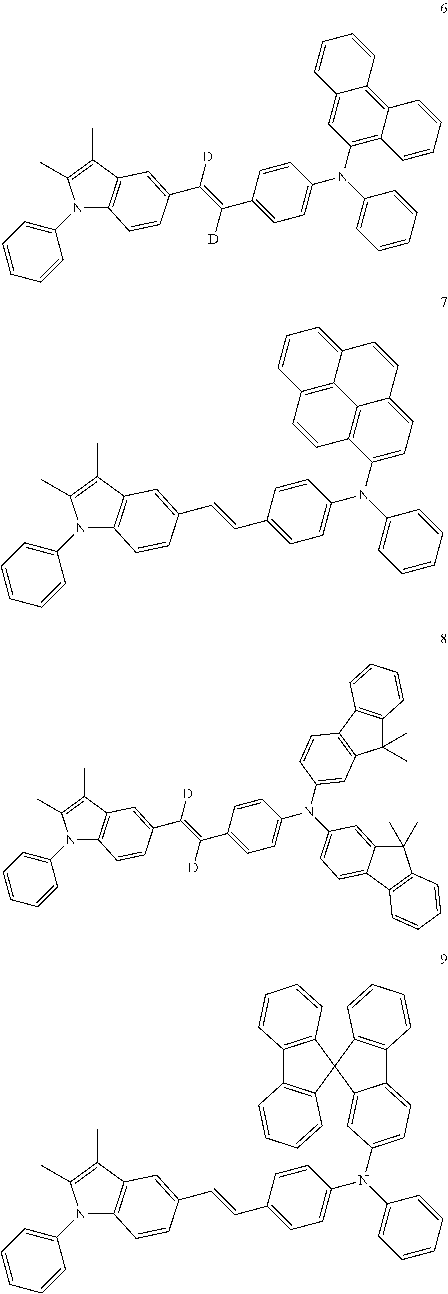 [SMILES: [2H]/C(=C(/[2H])c1ccc2c(c1)c(C)c(C)n2-c1ccccc1)c1ccc(N(c2ccccc2)c2cc3ccccc3c3ccccc23)cc1 ; Cc1c(C)n(-c2ccccc2)c2ccc(/C=C/c3ccc(N(c4ccccc4)c4ccc5ccc6cccc7ccc4c5c67)cc3)cc12 ; [2H]/C(=C(/[2H])c1ccc2c(c1)c(C)c(C)n2-c1ccccc1)c1ccc(N(c2ccc3c(c2)C(C)(C)c2ccccc2-3)c2ccc3c(c2)C(C)(C)c2ccccc2-3)cc1 ; Cc1c(C)n(-c2ccccc2)c2ccc(/C=C/c3ccc(N(c4ccccc4)c4ccc5c(c4)C4(c6ccccc6-c6ccccc64)c4ccccc4-5)cc3)cc12]

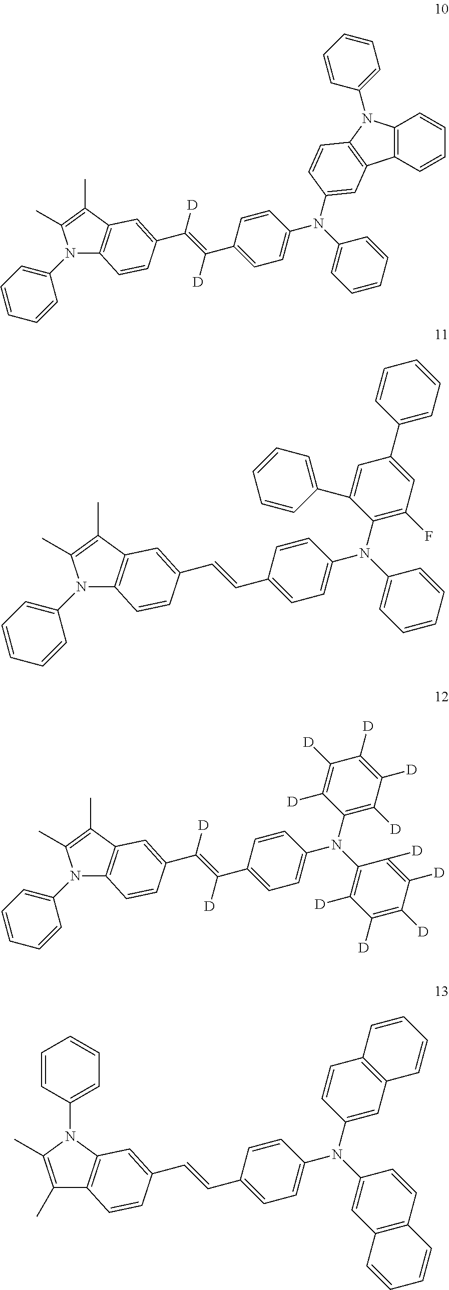 [SMILES: [2H]/C(=C(/[2H])c1ccc2c(c1)c(C)c(C)n2-c1ccccc1)c1ccc(N(c2ccccc2)c2ccc3c(c2)c2ccccc2n3-c2ccccc2)cc1 ; Cc1c(C)n(-c2ccccc2)c2ccc(/C=C/c3ccc(N(c4ccccc4)c4c(F)cc(-c5ccccc5)cc4-c4ccccc4)cc3)cc12 ; [2H]/C(=C(/[2H])c1ccc2c(c1)c(C)c(C)n2-c1ccccc1)c1ccc(N(c2c([2H])c([2H])c([2H])c([2H])c2[2H])c2c([2H])c([2H])c([2H])c([2H])c2[2H])cc1 ; Cc1c(C)n(-c2ccccc2)c2cc(/C=C/c3ccc(N(c4ccc5ccccc5c4)c4ccc5ccccc5c4)cc3)ccc12]

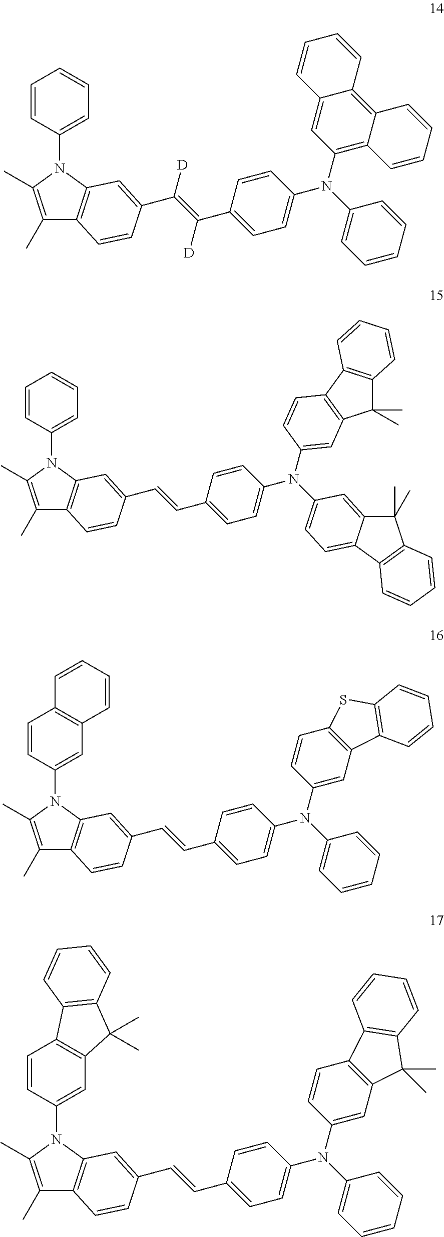

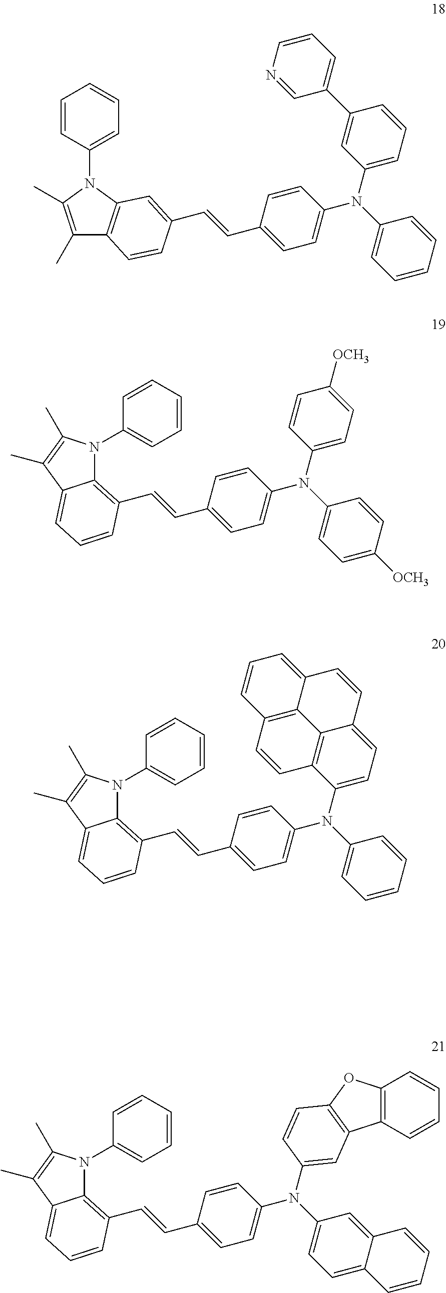 [SMILES: Cc1c(C)n(-c2ccccc2)c2cc(/C=C/c3ccc(N(c4ccccc4)c4cccc(-c5cccnc5)c4)cc3)ccc12 ; COc1ccc(N(c2ccc(/C=C/c3cccc4c(C)c(C)n(-c5ccccc5)c34)cc2)c2ccc(OC)cc2)cc1 ; Cc1c(C)n(-c2ccccc2)c2c(/C=C/c3ccc(N(c4ccccc4)c4ccc5ccc6cccc7ccc4c5c67)cc3)cccc12 ; Cc1c(C)n(-c2ccccc2)c2c(/C=C/c3ccc(N(c4ccc5ccccc5c4)c4ccc5oc6ccccc6c5c4)cc3)cccc12]

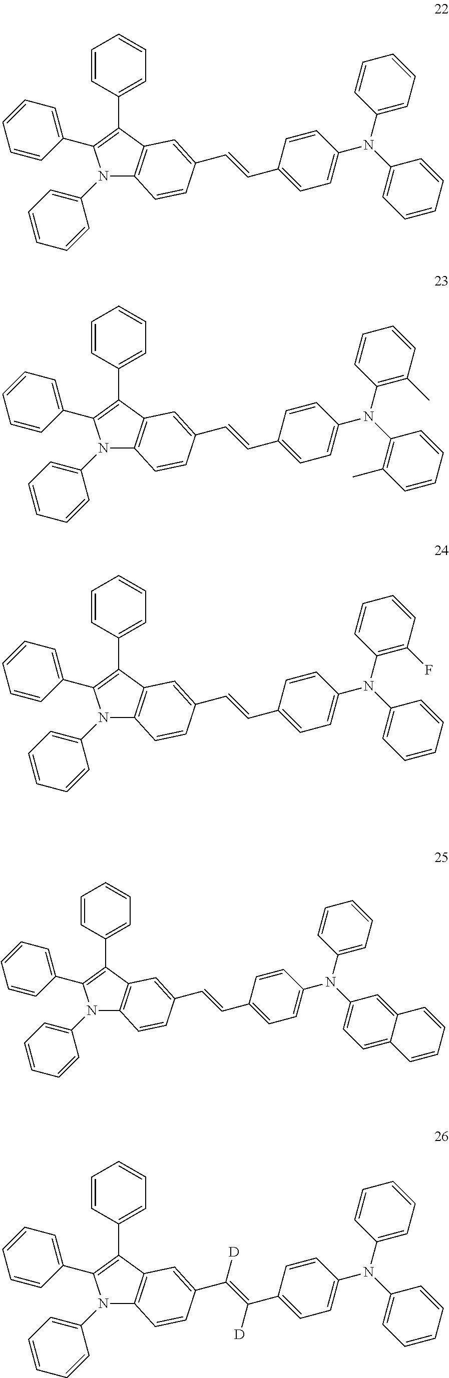 [SMILES: C(=C\c1ccc2c(c1)c(-c1ccccc1)c(-c1ccccc1)n2-c1ccccc1)/c1ccc(N(c2ccccc2)c2ccccc2)cc1 ; Cc1ccccc1N(c1ccc(/C=C/c2ccc3c(c2)c(-c2ccccc2)c(-c2ccccc2)n3-c2ccccc2)cc1)c1ccccc1C ; Fc1ccccc1N(c1ccccc1)c1ccc(/C=C/c2ccc3c(c2)c(-c2ccccc2)c(-c2ccccc2)n3-c2ccccc2)cc1 ; C(=C\c1ccc2c(c1)c(-c1ccccc1)c(-c1ccccc1)n2-c1ccccc1)/c1ccc(N(c2ccccc2)c2ccc3ccccc3c2)cc1 ; [2H]/C(=C(/[2H])c1ccc2c(c1)c(-c1ccccc1)c(-c1ccccc1)n2-c1ccccc1)c1ccc(N(c2ccccc2)c2ccccc2)cc1]

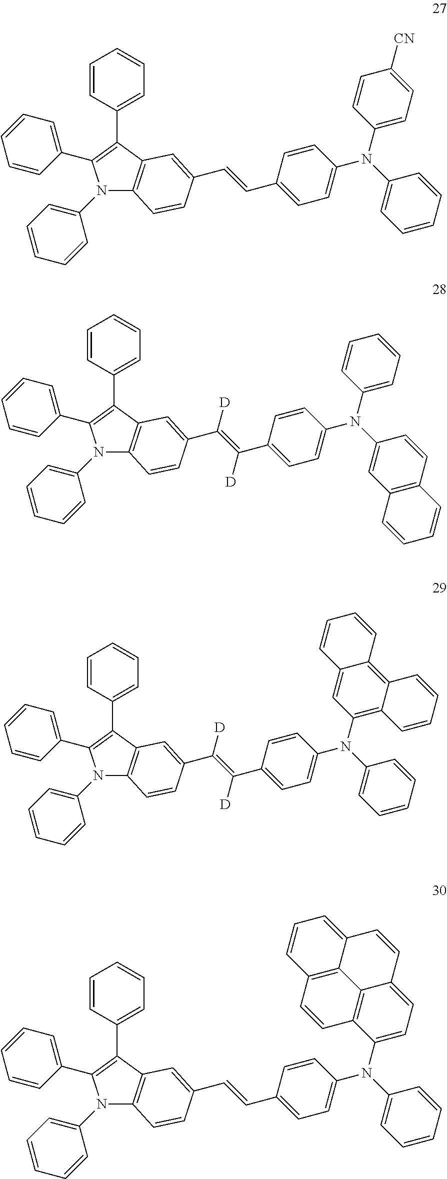 [SMILES: N#Cc1ccc(N(c2ccccc2)c2ccc(/C=C/c3ccc4c(c3)c(-c3ccccc3)c(-c3ccccc3)n4-c3ccccc3)cc2)cc1 ; [2H]/C(=C(/[2H])c1ccc2c(c1)c(-c1ccccc1)c(-c1ccccc1)n2-c1ccccc1)c1ccc(N(c2ccccc2)c2ccc3ccccc3c2)cc1 ; [2H]/C(=C(/[2H])c1ccc2c(c1)c(-c1ccccc1)c(-c1ccccc1)n2-c1ccccc1)c1ccc(N(c2ccccc2)c2cc3ccccc3c3ccccc23)cc1 ; C(=C\c1ccc2c(c1)c(-c1ccccc1)c(-c1ccccc1)n2-c1ccccc1)/c1ccc(N(c2ccccc2)c2ccc3ccc4cccc5ccc2c3c45)cc1]

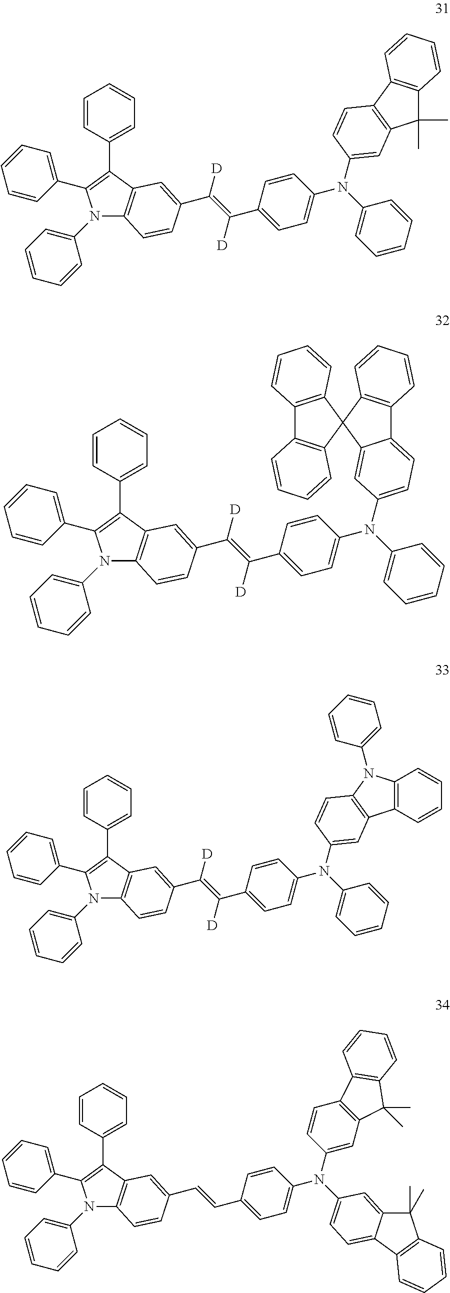 [SMILES: [2H]/C(=C(/[2H])c1ccc2c(c1)c(-c1ccccc1)c(-c1ccccc1)n2-c1ccccc1)c1ccc(N(c2ccccc2)c2ccc3c(c2)C(C)(C)c2ccccc2-3)cc1 ; [2H]/C(=C(/[2H])c1ccc2c(c1)c(-c1ccccc1)c(-c1ccccc1)n2-c1ccccc1)c1ccc(N(c2ccccc2)c2ccc3c(c2)C2(c4ccccc4-c4ccccc42)c2ccccc2-3)cc1 ; [2H]/C(=C(/[2H])c1ccc2c(c1)c(-c1ccccc1)c(-c1ccccc1)n2-c1ccccc1)c1ccc(N(c2ccccc2)c2ccc3c(c2)c2ccccc2n3-c2ccccc2)cc1 ; CC1(C)c2ccccc2-c2ccc(N(c3ccc(/C=C/c4ccc5c(c4)c(-c4ccccc4)c(-c4ccccc4)n5-c4ccccc4)cc3)c3ccc4c(c3)C(C)(C)c3ccccc3-4)cc21]

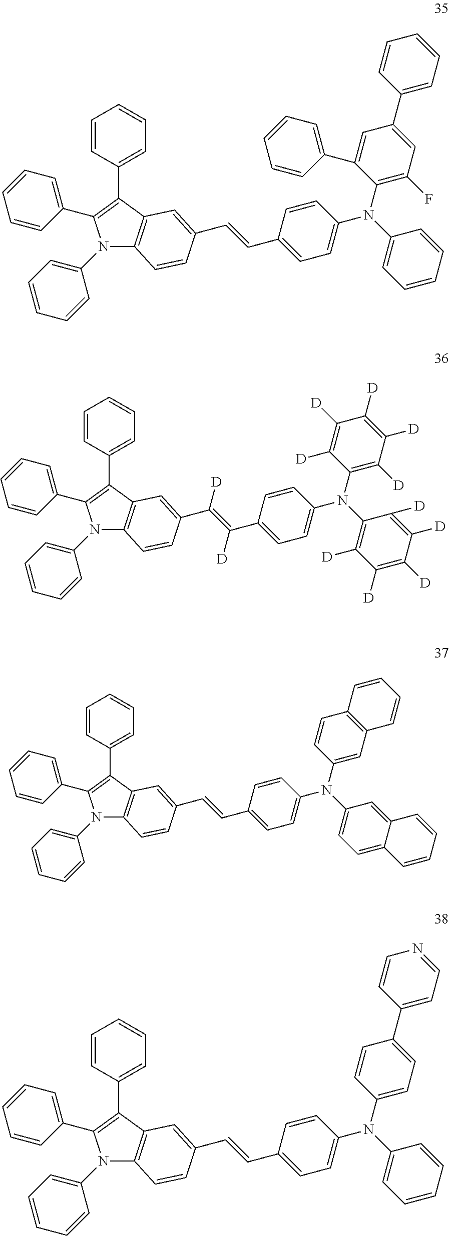 [SMILES: Fc1cc(-c2ccccc2)cc(-c2ccccc2)c1N(c1ccccc1)c1ccc(/C=C/c2ccc3c(c2)c(-c2ccccc2)c(-c2ccccc2)n3-c2ccccc2)cc1 ; [2H]/C(=C(/[2H])c1ccc2c(c1)c(-c1ccccc1)c(-c1ccccc1)n2-c1ccccc1)c1ccc(N(c2c([2H])c([2H])c([2H])c([2H])c2[2H])c2c([2H])c([2H])c([2H])c([2H])c2[2H])cc1 ; C(=C\c1ccc2c(c1)c(-c1ccccc1)c(-c1ccccc1)n2-c1ccccc1)/c1ccc(N(c2ccc3ccccc3c2)c2ccc3ccccc3c2)cc1 ; C(=C\c1ccc2c(c1)c(-c1ccccc1)c(-c1ccccc1)n2-c1ccccc1)/c1ccc(N(c2ccccc2)c2ccc(-c3ccncc3)cc2)cc1]

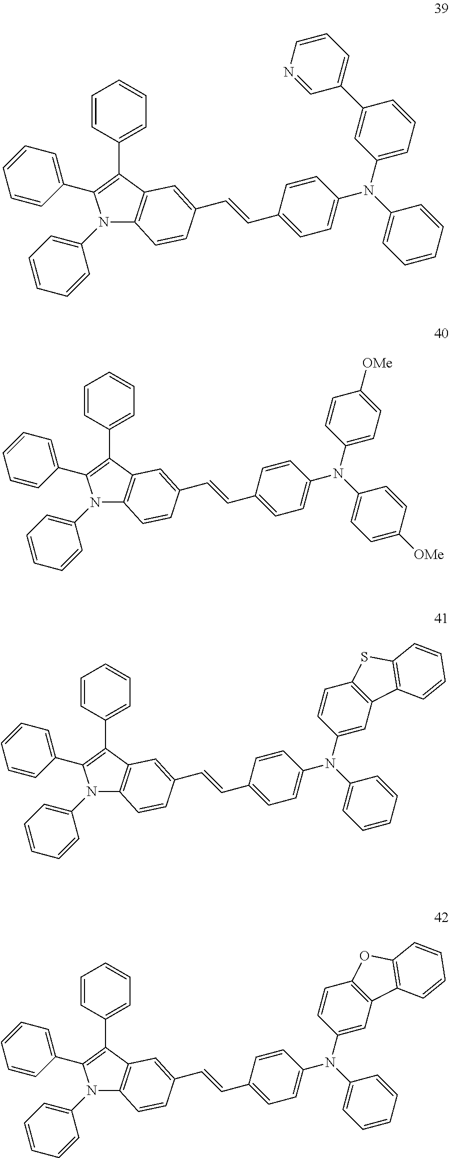 [SMILES: C(=C\c1ccc2c(c1)c(-c1ccccc1)c(-c1ccccc1)n2-c1ccccc1)/c1ccc(N(c2ccccc2)c2cccc(-c3cccnc3)c2)cc1 ; COc1ccc(N(c2ccc(/C=C/c3ccc4c(c3)c(-c3ccccc3)c(-c3ccccc3)n4-c3ccccc3)cc2)c2ccc(OC)cc2)cc1 ; C(=C\c1ccc2c(c1)c(-c1ccccc1)c(-c1ccccc1)n2-c1ccccc1)/c1ccc(N(c2ccccc2)c2ccc3sc4ccccc4c3c2)cc1 ; C(=C\c1ccc2c(c1)c(-c1ccccc1)c(-c1ccccc1)n2-c1ccccc1)/c1ccc(N(c2ccccc2)c2ccc3oc4ccccc4c3c2)cc1]

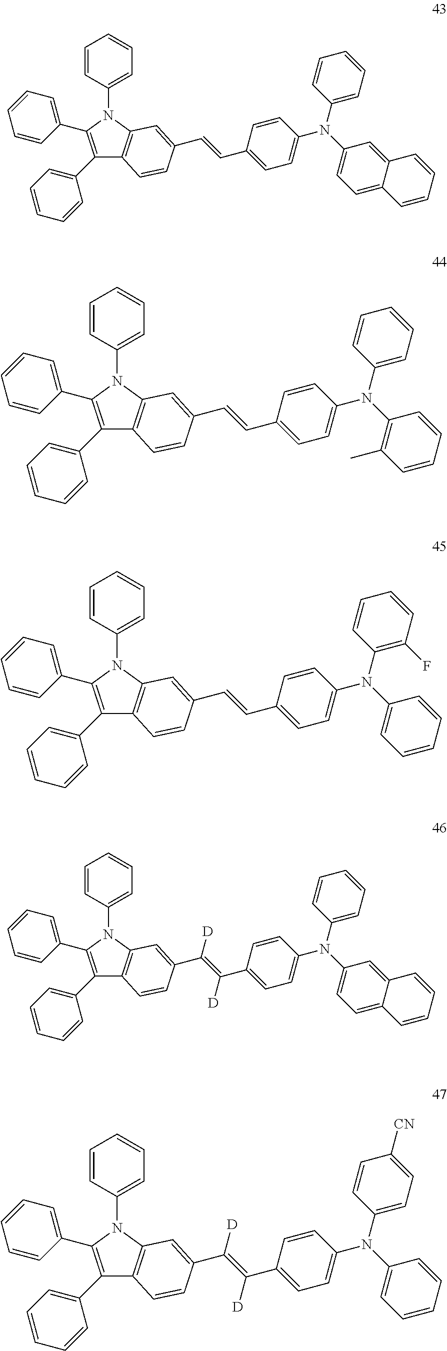 [SMILES: C(=C\c1ccc2c(-c3ccccc3)c(-c3ccccc3)n(-c3ccccc3)c2c1)/c1ccc(N(c2ccccc2)c2ccc3ccccc3c2)cc1 ; Cc1ccccc1N(c1ccccc1)c1ccc(/C=C/c2ccc3c(-c4ccccc4)c(-c4ccccc4)n(-c4ccccc4)c3c2)cc1 ; Fc1ccccc1N(c1ccccc1)c1ccc(/C=C/c2ccc3c(-c4ccccc4)c(-c4ccccc4)n(-c4ccccc4)c3c2)cc1 ; [2H]/C(=C(/[2H])c1ccc2c(-c3ccccc3)c(-c3ccccc3)n(-c3ccccc3)c2c1)c1ccc(N(c2ccccc2)c2ccc3ccccc3c2)cc1 ; [2H]/C(=C(/[2H])c1ccc2c(-c3ccccc3)c(-c3ccccc3)n(-c3ccccc3)c2c1)c1ccc(N(c2ccccc2)c2ccc(C#N)cc2)cc1]

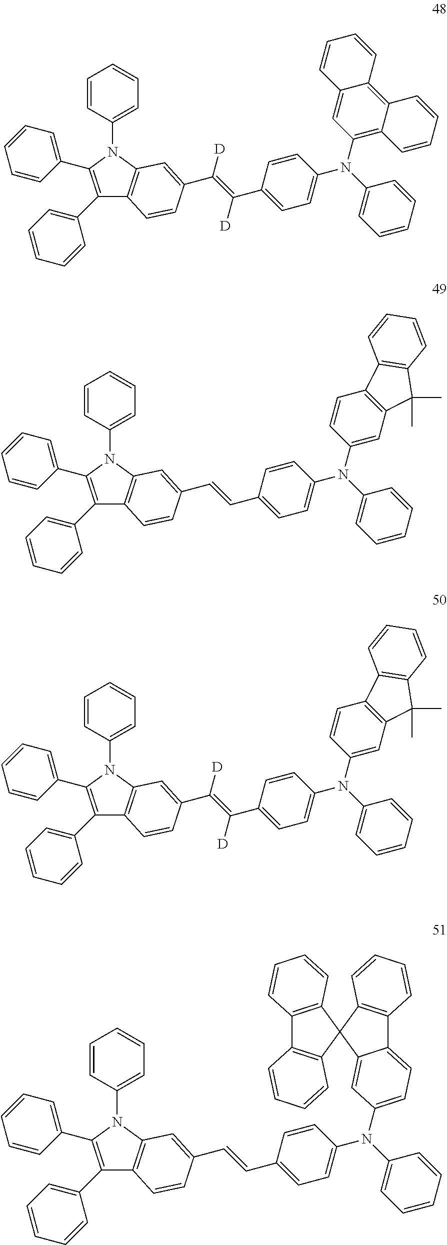 [SMILES: [2H]/C(=C(/[2H])c1ccc2c(-c3ccccc3)c(-c3ccccc3)n(-c3ccccc3)c2c1)c1ccc(N(c2ccccc2)c2cc3ccccc3c3ccccc23)cc1 ; CC1(C)c2ccccc2-c2ccc(N(c3ccccc3)c3ccc(/C=C/c4ccc5c(-c6ccccc6)c(-c6ccccc6)n(-c6ccccc6)c5c4)cc3)cc21 ; [2H]/C(=C(/[2H])c1ccc2c(-c3ccccc3)c(-c3ccccc3)n(-c3ccccc3)c2c1)c1ccc(N(c2ccccc2)c2ccc3c(c2)C(C)(C)c2ccccc2-3)cc1 ; C(=C\c1ccc2c(-c3ccccc3)c(-c3ccccc3)n(-c3ccccc3)c2c1)/c1ccc(N(c2ccccc2)c2ccc3c(c2)C2(c4ccccc4-c4ccccc42)c2ccccc2-3)cc1]

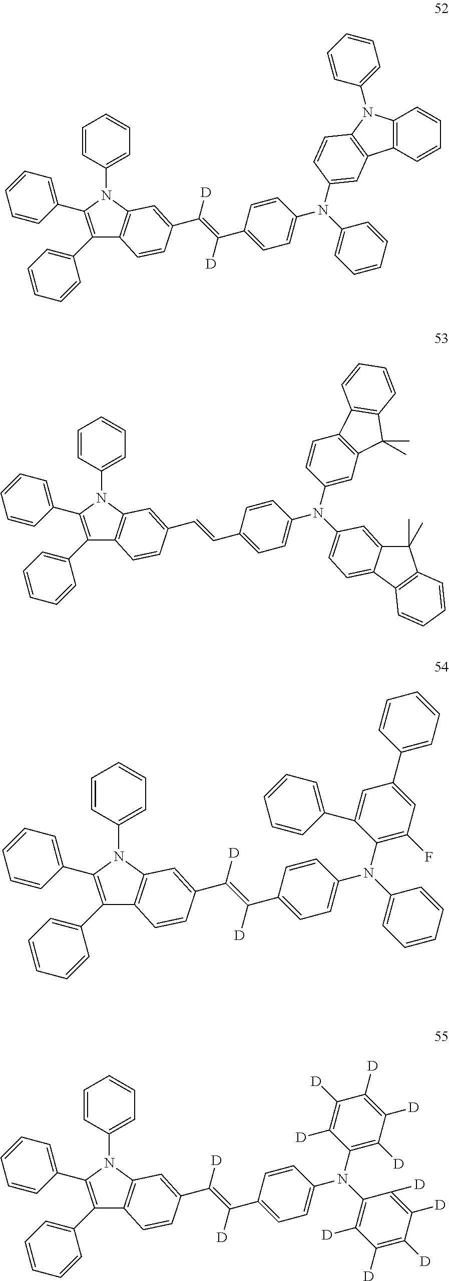

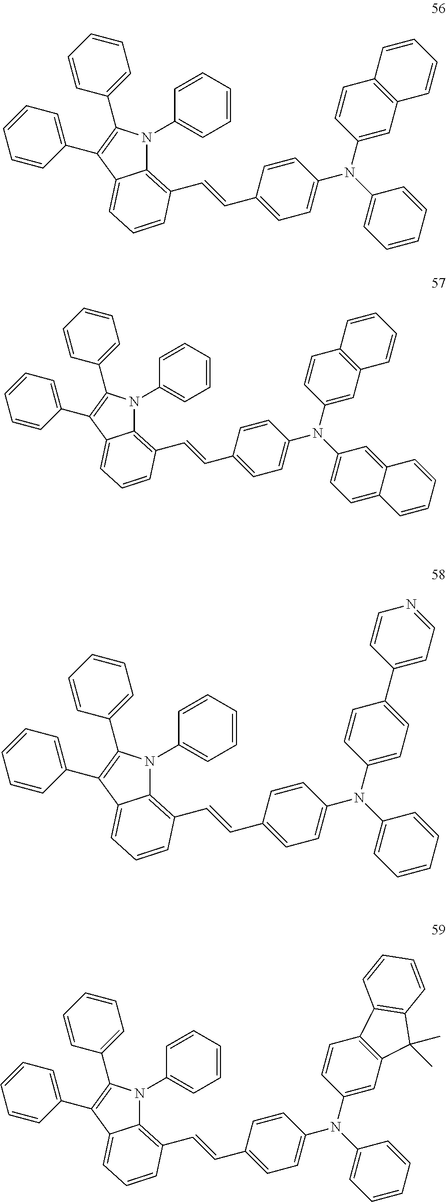 [SMILES: C(=C\c1cccc2c(-c3ccccc3)c(-c3ccccc3)n(-c3ccccc3)c12)/c1ccc(N(c2ccccc2)c2ccc3ccccc3c2)cc1 ; C(=C\c1cccc2c(-c3ccccc3)c(-c3ccccc3)n(-c3ccccc3)c12)/c1ccc(N(c2ccc3ccccc3c2)c2ccc3ccccc3c2)cc1 ; C(=C\c1cccc2c(-c3ccccc3)c(-c3ccccc3)n(-c3ccccc3)c12)/c1ccc(N(c2ccccc2)c2ccc(-c3ccncc3)cc2)cc1 ; CC1(C)c2ccccc2-c2ccc(N(c3ccccc3)c3ccc(/C=C/c4cccc5c(-c6ccccc6)c(-c6ccccc6)n(-c6ccccc6)c45)cc3)cc21]

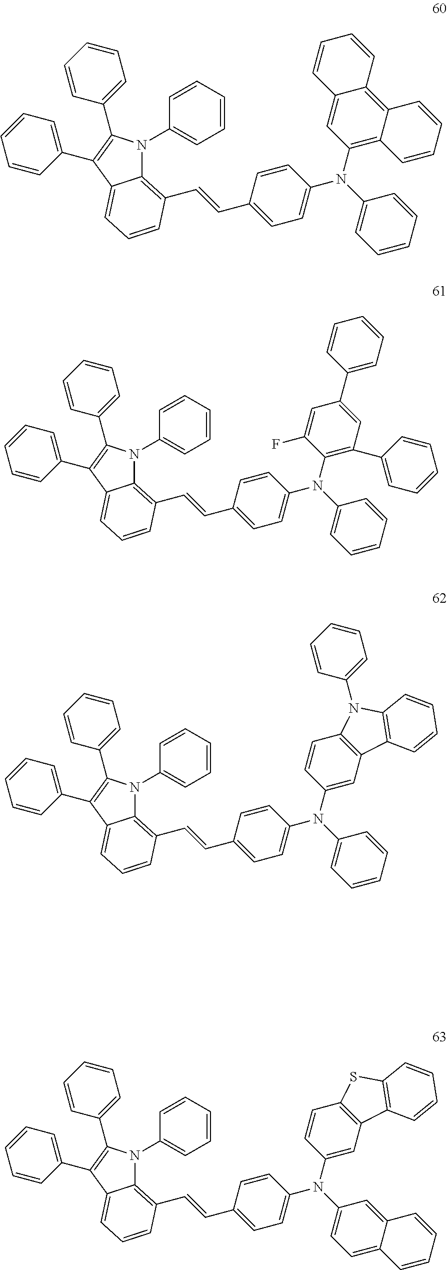 [SMILES: C(=C\c1cccc2c(-c3ccccc3)c(-c3ccccc3)n(-c3ccccc3)c12)/c1ccc(N(c2ccccc2)c2cc3ccccc3c3ccccc23)cc1 ; Fc1cc(-c2ccccc2)cc(-c2ccccc2)c1N(c1ccccc1)c1ccc(/C=C/c2cccc3c(-c4ccccc4)c(-c4ccccc4)n(-c4ccccc4)c23)cc1 ; C(=C\c1cccc2c(-c3ccccc3)c(-c3ccccc3)n(-c3ccccc3)c12)/c1ccc(N(c2ccccc2)c2ccc3c(c2)c2ccccc2n3-c2ccccc2)cc1 ; C(=C\c1cccc2c(-c3ccccc3)c(-c3ccccc3)n(-c3ccccc3)c12)/c1ccc(N(c2ccc3ccccc3c2)c2ccc3sc4ccccc4c3c2)cc1]

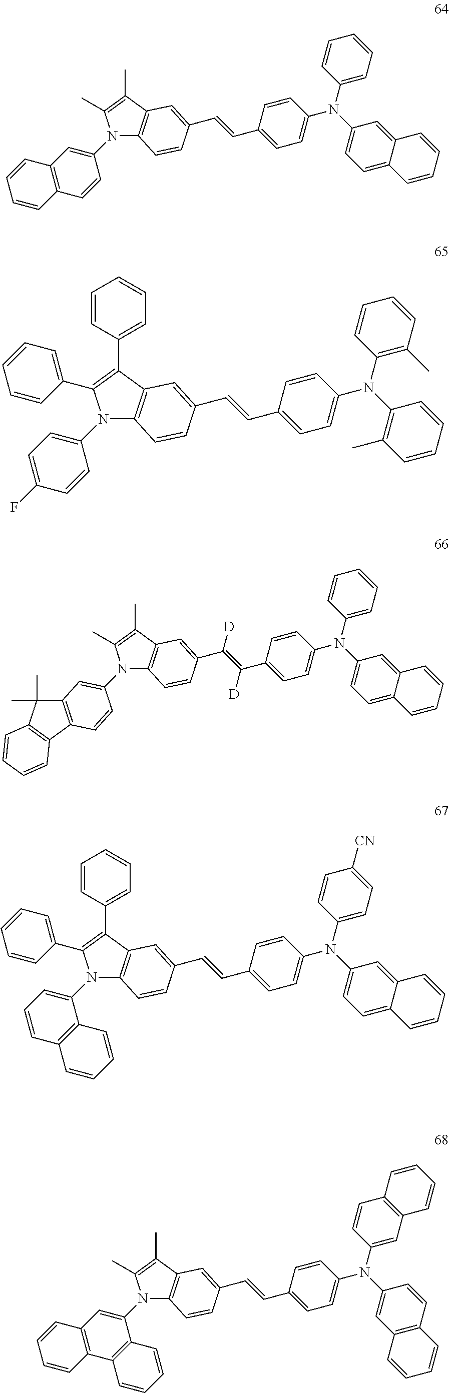 [SMILES: Cc1c(C)n(-c2ccc3ccccc3c2)c2ccc(/C=C/c3ccc(N(c4ccccc4)c4ccc5ccccc5c4)cc3)cc12 ; Cc1ccccc1N(c1ccc(/C=C/c2ccc3c(c2)c(-c2ccccc2)c(-c2ccccc2)n3-c2ccc(F)cc2)cc1)c1ccccc1C ; [2H]/C(=C(/[2H])c1ccc2c(c1)c(C)c(C)n2-c1ccc2c(c1)C(C)(C)c1ccccc1-2)c1ccc(N(c2ccccc2)c2ccc3ccccc3c2)cc1 ; N#Cc1ccc(N(c2ccc(/C=C/c3ccc4c(c3)c(-c3ccccc3)c(-c3ccccc3)n4-c3cccc4ccccc34)cc2)c2ccc3ccccc3c2)cc1 ; Cc1c(C)n(-c2cc3ccccc3c3ccccc23)c2ccc(/C=C/c3ccc(N(c4ccc5ccccc5c4)c4ccc5ccccc5c4)cc3)cc12]

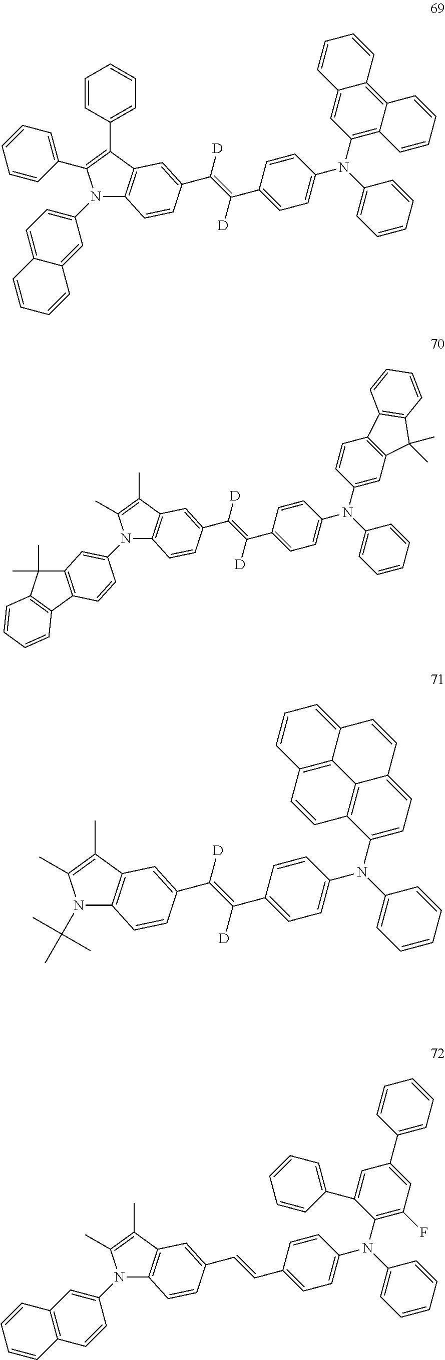

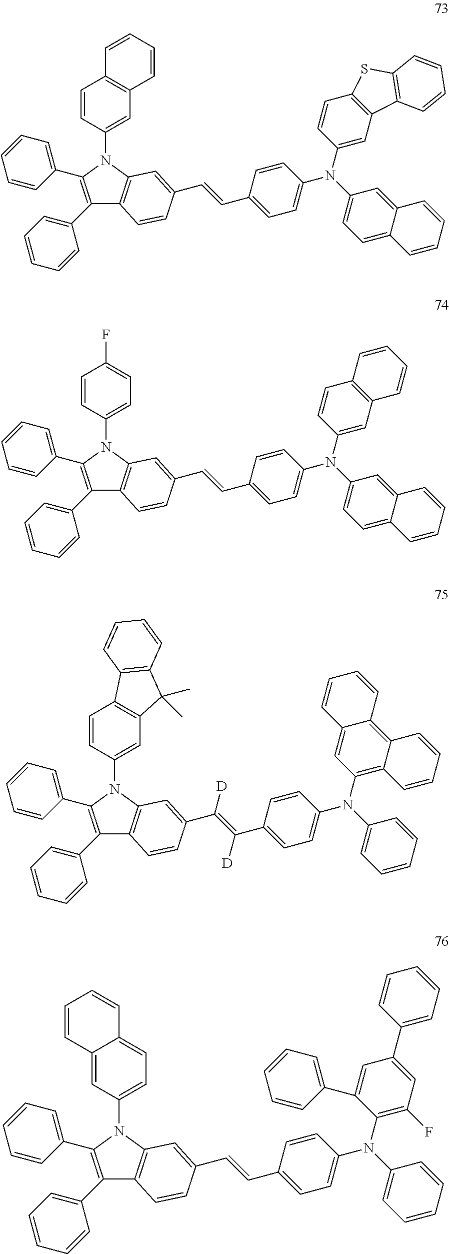 [SMILES: C(=C\c1ccc2c(-c3ccccc3)c(-c3ccccc3)n(-c3ccc4ccccc4c3)c2c1)/c1ccc(N(c2ccc3ccccc3c2)c2ccc3sc4ccccc4c3c2)cc1 ; Fc1ccc(-n2c(-c3ccccc3)c(-c3ccccc3)c3ccc(/C=C/c4ccc(N(c5ccc6ccccc6c5)c5ccc6ccccc6c5)cc4)cc32)cc1 ; [2H]/C(=C(/[2H])c1ccc2c(-c3ccccc3)c(-c3ccccc3)n(-c3ccc4c(c3)C(C)(C)c3ccccc3-4)c2c1)c1ccc(N(c2ccccc2)c2cc3ccccc3c3ccccc23)cc1 ; Fc1cc(-c2ccccc2)cc(-c2ccccc2)c1N(c1ccccc1)c1ccc(/C=C/c2ccc3c(-c4ccccc4)c(-c4ccccc4)n(-c4ccc5ccccc5c4)c3c2)cc1]

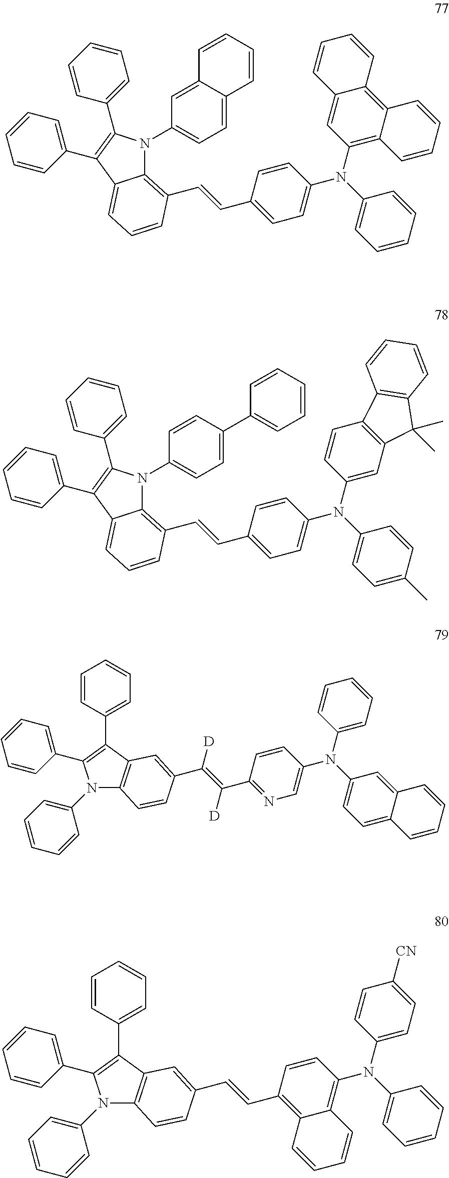 [SMILES: C(=C\c1cccc2c(-c3ccccc3)c(-c3ccccc3)n(-c3ccc4ccccc4c3)c12)/c1ccc(N(c2ccccc2)c2cc3ccccc3c3ccccc23)cc1 ; Cc1ccc(N(c2ccc(/C=C/c3cccc4c(-c5ccccc5)c(-c5ccccc5)n(-c5ccc(-c6ccccc6)cc5)c34)cc2)c2ccc3c(c2)C(C)(C)c2ccccc2-3)cc1 ; [2H]/C(=C(/[2H])c1ccc(N(c2ccccc2)c2ccc3ccccc3c2)cn1)c1ccc2c(c1)c(-c1ccccc1)c(-c1ccccc1)n2-c1ccccc1 ; N#Cc1ccc(N(c2ccccc2)c2ccc(/C=C/c3ccc4c(c3)c(-c3ccccc3)c(-c3ccccc3)n4-c3ccccc3)c3ccccc23)cc1]

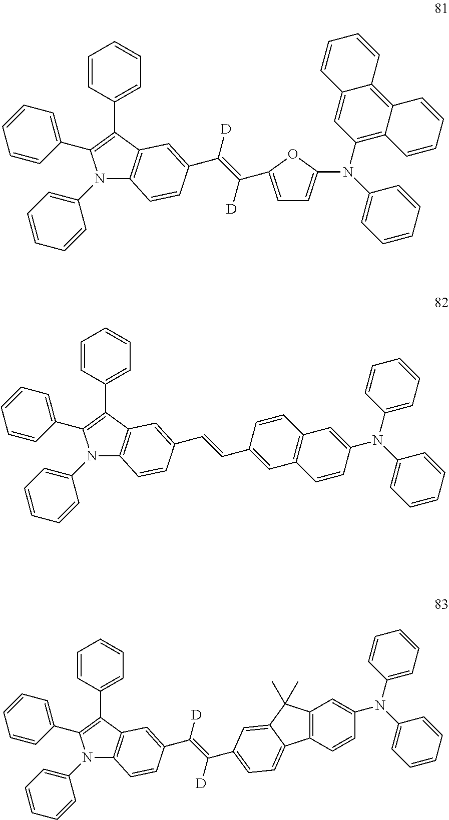 [SMILES: [2H]/C(=C(/[2H])c1ccc(N(c2ccccc2)c2cc3ccccc3c3ccccc23)o1)c1ccc2c(c1)c(-c1ccccc1)c(-c1ccccc1)n2-c1ccccc1 ; C(=C\c1ccc2c(c1)c(-c1ccccc1)c(-c1ccccc1)n2-c1ccccc1)/c1ccc2cc(N(c3ccccc3)c3ccccc3)ccc2c1 ; [2H]/C(=C(/[2H])c1ccc2c(c1)c(-c1ccccc1)c(-c1ccccc1)n2-c1ccccc1)c1ccc2c(c1)C(C)(C)c1cc(N(c3ccccc3)c3ccccc3)ccc1-2]

14. The heterocyclic compound of claim 1, wherein the hetero compound represented by Formula 1A is one of Compounds 10, 28, 35, 53, 67, 77 and 83 illustrated below:

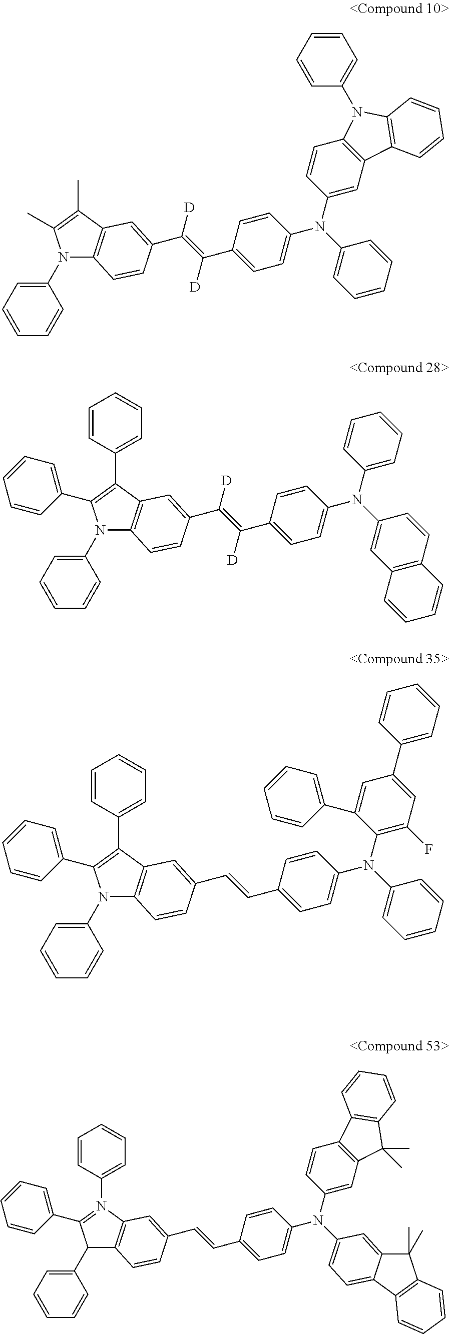

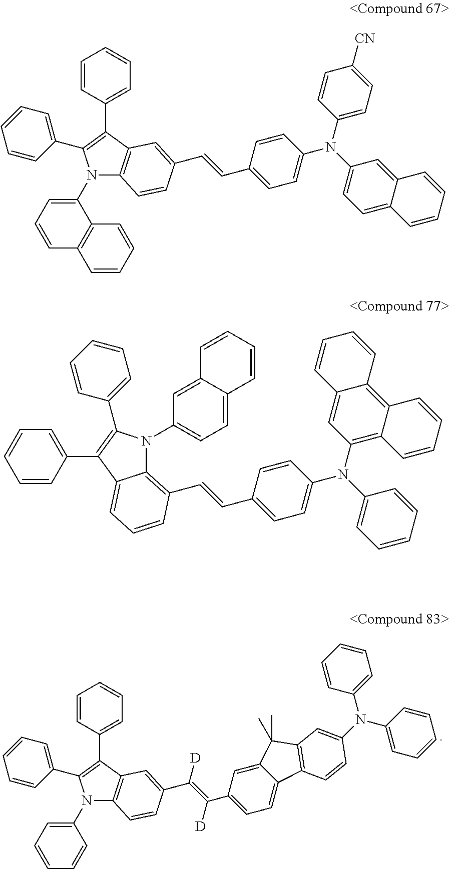 [SMILES: N#Cc1ccc(N(c2ccc(/C=C/c3ccc4c(c3)c(-c3ccccc3)c(-c3ccccc3)n4-c3cccc4ccccc34)cc2)c2ccc3ccccc3c2)cc1 ; C(=C\c1cccc2c(-c3ccccc3)c(-c3ccccc3)n(-c3ccc4ccccc4c3)c12)/c1ccc(N(c2ccccc2)c2cc3ccccc3c3ccccc23)cc1 ; [2H]/C(=C(/[2H])c1ccc2c(c1)c(-c1ccccc1)c(-c1ccccc1)n2-c1ccccc1)c1ccc2c(c1)C(C)(C)c1cc(N(c3ccccc3)c3ccccc3)ccc1-2]

15. An organic light-emitting diode comprising:
a first electrode;
a second electrode disposed facing the first electrode; and
an organic layer interposed between the first electrode and the second electrode, wherein the organic layer comprises at least one layer and one or more kinds of the heterocyclic compound of claim 1.

16. The organic light-emitting diode of claim 15, wherein the organic layer comprises at least one of a hole injection layer, a hole transport layer, a hole injection and transport layer having a hole injection capability and a hole transporting capability, an emission layer, an electron injection layer, an electron transport layer, and an electron injection and transport layer having an electron injection capability and an electron transporting capability.

17. The organic light-emitting diode of claim 16, wherein the organic layer comprises at least one of the emission layer, the hole injection layer, the hole transport layer, and the hole injection and transport layer, and at least one of the emission layer, the hole injection layer, the hole transport layer, and the hole injection and transport layer comprises the heterocyclic compound.

18. The organic light-emitting diode of claim 16, wherein the organic layer comprises an emission layer, the emission layer comprises a host and a dopant, and the heterocyclic compound is a fluorescent host of the emission layer.

19. The organic light-emitting diode of claim 16, wherein the organic layer comprises the emission layer, the emission layer comprises a host and a dopant, and the heterocyclic compound is a phosphorescent host of the emission layer.

20. The organic light-emitting diode of claim 16, wherein the organic layer comprises the emission layer, the emission layer comprises a host and a dopant, and the heterocyclic compound is a fluorescent dopant.

21. The organic light-emitting diode of claim 16, wherein the emission layer emits blue light.

22. The organic light-emitting diode of claim 16, wherein the organic layer comprises the emission layer, the emission layer comprises a host and a phosphorescent dopant.

23. The organic light-emitting diode of claim 16, wherein at least one of the hole injection layer, the hole transport layer, and the hole injection and transport layer further comprises, in addition to the heterocyclic compound, a charge-generating material.

24. The organic light-emitting diode of claim 23, wherein the charge-generating material is a p-type dopant.

25. The organic light-emitting diode of claim 17, wherein the organic layer comprises the electron transport layer, the electron transport layer comprises an electron transporting organic compound and a metal-containing material.

26. The organic light-emitting diode of claim 25, wherein the metal-containing material comprises a Li complex.

27. The organic light-emitting diode of claim 15, wherein at least one layer of the organic layer is formed by using a wet process.
